(19) United States
(12) Patent Application Publication
(10) Pub. No.: US 2021/0277428 A1
(43) Pub. Date: Sep. 9, 2021

(54) NOVEL MALONYL-COA BIOSENSOR BASED ON TYPE III POLYKETIDE SYNTHASE AND USE THEREOF

(71) Applicant: KOREA ADVANCED INSTITUTE OF SCIENCE AND TECHNOLOGY, Daejeon (KR)

(72) Inventors: Sang Yup Lee, Daejeon (KR); Dongsoo YANG, Daejeon (KR)

(21) Appl. No.: 16/477,897

(22) PCT Filed: Aug. 30, 2018
(86) PCT No.: PCT/KR2018/010087

§ 371 Date: Jul. 14, 2019

(30) Foreign Application Priority Data
Jun. 8, 2018 (KR) 10-2018-0066323

(51) Int. Cl. C12P 7/22; C12N 15/63; C12N 9/10; C12P 17/06; C12P 7/42; C12Q 1/00; C12Q 1/02; C12Q 1/48

(52) U.S. Cl. CPC C12P 7/22; C12N 15/63; C12N 9/1029; C12Q 1/48; C12P 7/42; C12Q 1/008; C12Q 1/025; C12P 17/06

(57) ABSTRACT

The present invention relates to a recombinant microorganism for malonyl-CoA detection in which a type III polyketide synthase-encoding gene is inserted in the genome or in which a recombinant vector containing the gene is introduced; a method of screening a malonyl-CoA production-inducing substance using the recombinant microorganism; a method of screening a gene which is involved in increased malonyl-CoA production; and a method comprising knocking down the gene, screened by the method, in a microorganism, thus increasing the production of malonyl-CoA in the microorganism, and producing a useful substance in the microorganism using malonyl-CoA as a precursor. The use of the biosensor according to the present invention provides single-step signal generation, utilization in various microorganisms, utilization in self-fluorescent microorganisms, a simple construction method, and a simple screening method. In addition, when the present invention is combined with high-throughput screening, it has advantages in that strains having increased malonyl-CoA producing ability can be screened very easily and rapidly (˜3 days) and can be applied directly to the malonyl-CoA-based production of useful compounds.

### TECHNICAL FIELD

The present invention relates to a novel malonyl-CoA biosensor based on type III polyketide synthase and the use thereof, and more particularly to a recombinant microorganism for malonyl-CoA detection into which a recombinant vector containing a type III polyketide synthase-encoding gene is introduced; a method of screening a malonyl-CoA production-inducing substance using the recombinant microorganism; a method of screening a gene which is involved in increased malonyl-CoA production; and a method comprising knocking down the gene, screened by the method, in a microorganism, thus increasing the production of malonyl-CoA in the microorganism, and producing a useful substance in the microorganism using malonyl-CoA as a precursor.

### BACKGROUND ART

Due to global environmental concerns, depletion of limited resources, and increased demand for environmentally friendly energy sources, the construction of cell factories based on reproducible organisms has been of increasing interest. These cell factories can produce desired metabolites (bioenergy, environmentally friendly chemical substances, new drug substances, etc.) through intracellular metabolic networks optimized for the production of the desired metabolites, and various molecular biology techniques are required for this technological process.

Among various substances which can be produced by microbial cell factories, malonyl-CoA is particularly important because it is a key precursor for producing very useful substances, such as polyketides, phenylpropanoids and biofuels. However, malonyl-CoA causes various essential metabolic reactions (such as fatty acid biosynthesis) in cells, and thus the amount of malonyl-CoA that can be utilized in metabolic engineering is very small. Furthermore, high-performance instruments such as LC-MS/MS are required to measure the intracellular concentration of malonyl-CoA, and a sampling method that takes a long time and requires very careful attention is required to measure malonyl-CoA which disappears after rapid production in cells and is sensitive to ambient environmental conditions. Therefore, transcription factor-based malonyl-CoA biosensors have been developed from previous studies in order to more quickly and easily measure the intracellular concentration of malonyl-CoA (Xu P, Li L, Zhang F, Stephanopoulos G, Koffas M (2014), Proc Natl Acad Sci USA 111:11299-11304; Li S, Si T, Wang M, Zhao H (2015), ACS Synth Biol 4:1308-1315). However, the fluorescent protein-based sensors based on transcription factors as described above have disadvantages in that they have to undergo various signaling steps and cannot be used in self-fluorescent microorganisms such as Pseudomonas.

Accordingly, the present inventors have made efforts to solve the above-described problems, and as a result, have developed a novel malonyl-CoA biosensor based on type III polyketide synthase, which utilizes the property that type III polyketide synthases, including RppA, convert malonyl-CoA into a colored substance through a single-step reaction, and have found that the use of the biosensor can easily detect malonyl-CoA, makes it possible to screen a malonyl-CoA-producing strain, a malonyl-CoA production-inducing substance, and a gene which is involved in increased malonyl-CoA production, and makes it possible to easily construct a method of producing various useful substances using malonyl-CoA as either a substrate or a precursor, thereby completing the present invention.

### DISCLOSURE OF INVENTION

### Technical Problem

It is an object of the present invention to provide a novel biosensor for malonyl-CoA detection and various methods of using the biosensor, and a method of increasing the production of malonyl-CoA in the microorganism by use of the methods, and producing a useful substance in the microorganism using malonyl-CoA as a substrate or a precursor.

### Technical Solution

To achieve the above object, the present invention provides a recombinant microorganism for malonyl-CoA detection in which a type III polyketide synthase-encoding gene is inserted in the genome of the microorganism or into which a recombinant vector containing the type III polyketide synthase-encoding gene is introduced.

The present invention also provides a method for screening a malonyl-CoA production-inducing substance, comprising the steps of:

(a) culturing the above-described recombinant microorganism;

(b) adding a candidate substance to the recombinant microorganism;

(c) comparing the color of a culture supernatant of the recombinant microorganism after addition of the candidate substance with the color of a culture supernatant of the recombinant microorganism without addition of the candidate substance; and

(d) selecting the candidate substance as the malonyl-CoA production-inducing substance, when the culture supernatant of the recombinant microorganism after addition of the candidate substance shows a deeper red color than the culture supernatant of the recombinant microorganism without addition of the candidate substance.

The present invention also provides a method for screening a gene involved in increased malonyl-CoA production, the method comprising the steps of:

(a) introducing a gene regulation library, which changes gene expression in a recombinant microorganism, into the above-described recombinant microorganism, thereby constructing a recombinant microorganism library in which gene expression in the recombinant microorganism is changed;

(b) culturing the constructed recombinant microorganism library and the recombinant microorganism into which the gene regulation library is not introduced, measuring the absorbance of the culture supernatants, and comparing the color of the culture supernatants; and

(c) selecting the gene introduced in the recombinant microorganism library, when the culture supernatant of the recombinant microorganism library shows a deeper red color than the culture supernatant of the recombinant microorganism into which the gene regulation library is not introduced.

The present invention also provides a method for producing a recombinant microorganism having increased malonyl-CoA producing ability, the method comprising regulating expression of the gene, screened by the above-described method, in a microorganism inherently having malonyl-CoA producing ability or externally introduced with malonyl-CoA producing ability.

The present invention also provides a recombinant microorganism having increased malonyl-CoA producing ability wherein expression of one or more genes selected from the group consisting of the following genes:

fabF (3-oxoacyl-[acyl-carrier-protein] synthase II);

yfcY (beta-ketoacyl-CoA thiolase);

xapR (transcriptional activator of xapAB);

cytR (transcriptional repressor for deo operon, udp, cdd, tsx, nupC and nupG);

fabH (3-oxoacyl-[acyl-carrier-protein] synthase III);

mqo (malate dehydrogenase);

yfiD (pyruvate formate lyase subunit);

fmt (10-formyltetrahydrofolate:L-methionyl-tRNA(fMet)N-formyltransferase);

pyrF (orotidine-5′-phosphate decarboxylase);

araA (L-arabinose isomerase);

fadR (negative regulator for fad regulon and positive regulator of fabA);

pabA (aminodeoxychorismate synthase, subunit II);

purB (adenylosuccinate lyase); and

hycI (protease involved in processing C-terminal end of HycE), in a microorganism having malonyl-CoA producing ability is decreased compared to that in a wild-type microorganism.

The present invention also provides a method of producing a useful substance using malonyl-CoA as a substrate or an intermediate, the method comprising the steps of:

(a) constructing a recombinant microorganism in which a gene involved in production of the useful substance is additionally introduced, or expression of the gene is increased, or the gene involved in production of the useful substance is additionally deleted, or expression of the gene is decreased;

(b) culturing the constructed microorganism; and

(c) recovering the useful substance from the cultured microorganism.

The present invention also provides a recombinant microorganism for producing 6-methylsalicylic acid in which genes that encode 6-methylsalicylic acid synthase (6MSAS) and 4′-phosphopantetheinyl transferase (Sfp) are additionally introduced in the above-described recombinant microorganism or expression of the genes is increased.

The present invention also provides a method of producing 6-methylsalicylic acid, the method comprising the steps of:

(a) culturing the above-described recombinant microorganism; and

(b) recovering the 6-methylsalicylic acid from the cultured microorganism.

The present invention also provides a recombinant microorganism for producing aloesone in which a gene that encodes aloesone synthase is additionally introduced in the above-described recombinant microorganism or expression of the gene is increased.

The present invention also provides a method of producing aloesone, the method comprising the steps of:

(a) culturing the above-described recombinant microorganism; and

(b) recovering the aloesone from the cultured microorganism.

The present invention also provides a recombinant microorganism for producing resveratrol in which genes that encode tyrosine ammonia-lyase (TAL), 4-coumarate:CoA ligase (4CL) and stilbene synthase (STS) are additionally introduced in the above-described recombinant microorganism or expression of the genes is increased.

The present invention also provides a method of producing resveratrol, the method comprising the steps of:

(a) culturing the above-described recombinant microorganism; and

(b) recovering the resveratrol from the cultured microorganism.

The present invention also provides a recombinant microorganism for producing naringenin in which genes that encode tyrosine ammonia-lyase (TAL), 4-coumarate:CoA ligase (4CL), chalcone synthase (CHS), and chalcone isomerase (CHI) are additionally introduced in the above-described recombinant microorganism or expression of the genes is increased.

The present invention also provides a method of producing naringenin, the method comprising the steps of:

(a) culturing the above-described recombinant microorganism; and

(b) recovering the naringenin from the cultured microorganism.

### BRIEF DESCRIPTION OF THE DRAWINGS

FIG. 1 shows the operational mechanism of a malonyl-CoA biosensor based on type III polyketide synthase (RppA). (A) RppA can convert five molecules of malonyl-CoA into one molecule of red-colored flaviolin. At this time, RppA is responsible for the conversion of malonyl-CoA to 1,3,6,8-tetrahydroxynaphthalene (THN), which is then spontaneously converted to flaviolin. (B) As the red color becomes deeper by the RppA biosensor, the intracellular level of malonyl-CoA is higher.

FIG. 2 shows flaviolin biosynthesis. (A) Flaviolin production by rppA from five different strains. (B) Production of flaviolin confirmed by LC-MS and MS/MS analysis. (C) Optimization of flaviolin production. Flaviolin production was increased by optimization of 5′UTR. (D) Examination of the applicability of an RppA biosensor. Changes in signals due to changes in intracellular levels of malonyl-CoA by an indirect method using cerulenin. RppA⁺ denotes an RppA-expressing strain, and RppA⁻ denotes a strain that does not express RppA.

FIG. 3 shows the applicability and scalability of an RppA biosensor. (A) Flaviolin production increases as the amount of cerulenin added increases, and (B) this can be confirmed even by the naked eyes. (C) Flaviolin production from all 16 E. coli strains is possible. (D) In the case of an RppA-expressing strain (RppA+) and a control strain (RppA−) that does not express RppA, the noise from the control at 340 nm (indicated by the arrow) was the lowest and the signal from the RppA+ strain was the strongest, and thus the absorbance at 340 nm was used as the signal of an RppA biosensor.

FIG. 4 shows characterization of three type III polyketide synthases (AaOKS, AaPKS4, and AaPKS5) as malonyl-CoA biosensors. (A) Extracted ion chromatograms of culture supernatants of E. coli BL21(DE3) strains expressing AaOKS, AaPKS4 and AaPKS5 generated by LC-MS (negative scan mode). The products predicted from the chromatograms are as follows: 5,7-dihydroxy-2-methylchromone (m/z 191); aloesone (m/z 231); SEK4 and SEK4b (m/z 317). (B) shows the colors of culture supernatants of E. coli strains expressing AaOKS, AaPKS4 and AaPKS5 when different concentrations of cerulenin was added to the medium, and (C) shows the optical density (absorbance) of the culture supernatants at different wavelengths. CT, control strain (BL21 (DE3) pET-30a(+)). At this time, the signal of malonyl-CoA biosensor strains at 300 nm was the strongest compared to the control, and thus in the case of AaOKS, AaPKS4 and AaPKS5, the absorbance at 300 nm was used as the signal. The data points represent the average values of three biological replicates.

FIG. 5 indicates that three type III polyketide synthases (AaOKS, AaPKS4, and AaPKS5) can be used as malonyl-CoA biosensors. (A) AaOKS, (B) AaPKS4, and (C) AaPKS5 indicate increased normalized signals caused by an increase in the malonyl-CoA level (controlled by addition of cerulenin). Each signal indicates each case that harbors or does not harbor PKS.

FIG. 6 shows the results of testing an RppA biosensor in Pseudomonas putida, Corynebacterium glutamicum, and Rhodococcus opacus. (A) shows the production of flaviolin when the indicated plasmid was transformed into a P. putida strain. (B) shows the cerulenin concentration-dependent signal intensity of a P. putida pBBR1-rppA sensor strain, and (C) shows relative flaviolin production at this time. (D) shows the production of flaviolin when the indicated plasmid was transformed into a C. glutamicum strain. (E) shows the cerulenin concentration-dependent signal intensity of a C. glutamicum pCES-His-rppA sensor strain, and (F) shows relative flaviolin production at this time. (G) shows the color of medium when the indicated plasmid was transformed into an R. opacus strain.

FIG. 7 shows a process of screening strains, which show increased malonyl-CoA production, by high-throughput screening using an RppA biosensor.

FIG. 8 shows the initial results of screening strains which show increased malonyl-CoA levels when an E. coli genome-scale synthetic control sRNA library was introduced and high-throughput screening was performed using an RppA biosensor.

FIG. 9 shows signals generated when 26 initially screened synthetic-control sRNAs involved in increased malonyl-CoA production were transformed into E. coli sensor strains. At this time, 14 synthetic-control sRNAs showing an increase in signal of 70% or more compared to the control were selected as final targets.

FIG. 10 shows FVSEOF simulation performed to identify increased malonyl-CoA production. (A) shows the biosynthetic pathway related to malonyl-CoA biosynthesis. Letters in bold denote reactions, and plain letters denote metabolites. Grey arrows denote metabolic fluxes, and red arrows denote metabolic fluxes identified as overexpression targets. (B) shows biosensor signals appearing when overexpressing the gene targets identified by FVSEOF. (C) shows lists the gene targets identified by FVSEOF.

FIG. 11 shows the biosynthetic pathway of 6-methylsalicylic acid. Here, red X denotes knocked-out genes, and blue X denotes knocked-down genes. bla, beta-lactamase gene; kan^{R}, kanamycin-resistance gene; p15A, replication origin; ColE1, replication origin; P_{tac}, tac promoter; P_{BAD}, arabinose-inducible promoter; P_{R}, P_{R} promoter; rrnB, rrnBT1T2 terminator; T1/TE, terminator. Gly, glycerol; Gly-3P, glycerol 3-phosphate; DHA, dihydroxyacetone; DHAP, dihydroxyacetone phosphate; G3P, glyceraldehyde 3-phosphate; 1,3BPG, 1,3-bisphosphoglycerate; 3PG, 3-phosphoglycerate; 2PG, 2-phosphoglycerate; PEP, phosphoenolpyruvate; PYR, pyruvate; OAA, oxaloacetate; E4P, D-erythrose 4-phosphate; DAHP, 3-deoxy-D-arabinoheptulosonate 7-phosphate; CHOR, chorismate; AcCoA, acetyl-CoA; MalCoA, malonyl-CoA; 6MSAS, 6MSA synthase; KS, ketosynthase; AT, acyltransferase; DH, dehydratase; KR, ketoreductase; ACP, acyl carrier protein.

FIG. 12 shows different gene expression cassettes for production of 6-methylsalicylic acid. (A) shows the plasmid pTac-Pg6MSAS-sfp, and (B) shows sfp expressed with the plasmid pTac-Pg6MSAS on the genome of an E. coli BAP1 strain. (C) shows the results of SDS-PAGE performed to analyze the expression of 6-methylsalicylic acid synthase (Pg6MSAS) and 4′-phosphopantetheinyl transferase (Sfp). Pg6MSAS (188 kDa), Sfp (26.1 kDa).

FIG. 13 shows the production of 6-methylsalicylic acid from modified E. coli strains. (A) shows the production of 6-methylsalicylic acid from various concentrations of glucose/glycerol in a BL21-based strain and a BAP1-based strain. (B) shows the results of LC-MS analysis of 6-methylsalicylic acid produced from E. coli, and (C) shows the results of LC-MS analysis of a commercially available 6-methylsalicylic acid compound. (D) shows test-tube scale 6-methylsalicylic acid production in 16 E. coli strains. Blue sections indicate a production of 1 mg/L or more. (E) shows time-course 6-methylsalicylic acid production in an E. coli BL21(DE3) pTac-Pg6MSAS-sfp pWAS-anti-pabA (pabA-knockdown sRNA plasmid) strain. (F) shows time-course 6-methylsalicylic acid production in a BAP1 pTac-Pg6MSAS pWAS-anti-pabA strain. (G) shows fed-batch fermentation of the same strain. The red arrow denotes the onset of IPTG induction. In (E), (F) and (G), the blue lines and points denote cell growth (OD₆₀₀), and the red lines and points denote 6-methylsalicylic acid concentrations.

FIG. 14 shows 6-methylsalicylic acid production when 14 synthetic control sRNAs selected in Example 2.1 were introduced into 6 screened E. coli strains and test tube-scale culture of the produced strains was performed.

The left graph of FIG. 15 shows the concentration of 6-methylsalicylic acid that appeared in flask culture when the genes at the bottom of the left graph were overexpressed in E. coli BAP1 pTac-Pg6MSAS pWAS-anti-pabA, a strain showing the highest 6-methylsalicylic acid productivity in FIG. 12. The right graph shows the results of fed-batch fermentation of E. coli BAP1 pTac-Pg6MSAS pWAS-anti-pabA pBBR1-accBCD1, a strain showing the highest productivity.

FIG. 16 shows the aloesone biosynthetic pathway. Here, red X denotes knocked-out genes, and blue X denotes knocked-down genes. Glc, glucose; G6P, glucose 6-phosphate; F6P, fructose 6-phosphate; F1,6BP, fructose 1,6-bisphosphate; DHAP, dihydroxyacetone phosphate; G3P, glyceraldehyde 3-phosphate; 1,3BPG, 1,3-bisphosphoglycerate; 3PG, 3-phosphoglycerate; 2PG, 2-phosphoglycerate; PEP, phosphoenolpyruvate; PYR, pyruvate; OAA, oxaloacetate; E4P, D-erythrose 4-phosphate; DAHP, 3-deoxy-D-arabinoheptulosonate 7-phosphate; CHOR, chorismate; AcCoA, acetyl-CoA; MalCoA, malonyl-CoA; ALS, aleosone synthase. bla, beta-lactamase gene; spc^{R}, spectinomycin-resistance gene; CDF, replication origin; ColE1, replication origin; P_{T7}, T7 promoter; P_{BAD}, arabinose-inducible promoter; PR, PR promoter; rrnB, rrnBT1T2 terminator; T1/TE, terminator.

FIG. 17 shows aloesone production in modified E. coli strains. (A) shows the results of SDS-PAGE performed to analyze the expression of aloesone synthase (RpALS, 43 kDa) and aloesone synthase (AaPKS3, 44 kDa). (B) shows the production of aloesone when RpALS/AaPKS3 was expressed using glucose/glycerol as a carbon source. (C) shows the LC-MS and MS/MS spectra of aloesone produced in E. coli. (D) shows time-course aloesone production in a BL21(DE3) pCDF-RpALS strain, and (E) shows time-course aloesone production in a BL21(DE3) pCDF-RpALS pWAS-anti-pabA strain. In (D) and (E), the blue lines and points depict cell growth (OD₆₀₀), and the red lines and points depict the aloesone concentration. (F) shows the results of test tube-scale culture of strains produced by introducing 14 synthetic-control sRNAs, selected in Example 2.1, into two types of E. coli strains.

FIG. 18 shows the concentration of aloesone that appeared in flask culture when the genes at the bottom of the graph were overexpressed in BL21(DE3) pCDF-RpALS pWAS-anti-pabA, a strain showing the highest aloesone productivity in FIG. 17(E).

FIG. 19 shows the resveratrol biosynthetic pathway. Here, red X denotes knocked-out genes, and blue X denotes knocked-down genes. Black arrows along with genes written in bold letters denote overexpressed metabolic fluxes. Red dotted lines (along with − signs in circles) indicate transcriptional repression. Black dotted lines (along with + signs in circles) indicate transcriptional activation. Gly, glycerol; Gly-3P, glycerol 3-phosphate; DHA, dihydroxyacetone; DHAP, dihydroxyacetone phosphate; G3P, glyceraldehyde 3-phosphate; 1,3BPG, 1,3-bisphosphoglycerate; 3PG, 3-phosphoglycerate; 2PG, 2-phosphoglycerate; PEP, phosphoenolpyruvate; PYR, pyruvate; OAA, oxaloacetate; E4P, D-erythrose 4-phosphate; DAHP, 3-deoxy-D-arabinoheptulosonate 7-phosphate; SHIK, shikimate; CHOR, chorismate; PPHN, prephenate; HPP, 4-hydroxyphenylpyruvate; FOR, formate; AcCoA, acetyl-CoA; MalCoA, malonyl-CoA. bla, beta-lactamase gene; kan^{R}, kanamycin-resistance gene; spc^{R}, spectinomycin-resistance gene; p15A, replication origin; ColE1, replication origin; CDF, replication origin; P_{tac}, tac promoter; P_{BAD}, arabinose-inducible promoter; P_{R}, P_{R} promoter; P_{trc}, trc promoter; rrnB, rrnBT1T2 terminator; T1/TE, terminator.

FIG. 20 shows the production of p-coumaric acid and resveratrol by modified E. coli strains. (A) shows the production of p-coumaric acid by various plasmids constructed to examine the effects of N-terminal protein tags. Each of the vectors except for pTY13-HisTAL was transformed into a BTY5.13 strain, and pTY13-HisTAL was transformed into a BTY5 strain. (B) shows the results of SDS-PAGE performed to analyze the expression of each protein. At4CL1m (61.1 kDa), At4CL3 (61.3 kDa), At4CL4 (62.6 kDa), Sc4CLm (55.3 kDa), STS (42.8 kDa). CT depicts a control. M depicts a protein size marker. (C) shows the results of resveratrol production by combinations of STS and different 4CLs. (D) shows the production of resveratrol by different plasmid construction strategies. Here, 1 to 5 depict plasmids, and each of the plasmids is as follows. 1, pTac-VvSTS-At4CL1m; 2, pTac-At4CL1m-opr-VvSTS (expressing two genes in an operon); 3, pTac-At4CL1m-fus-VvSTS (expressing a fusion protein of At4CL1m and STS); 4 and 5, pTacCDF-VvSTS-At4CL1m. Here, the plasmids corresponding to 1 to 4 were introduced into E. coli BL21(DE3) and were cultured in the presence of 2 mM p-coumaric acid and 20 g/L glycerol. The plasmid corresponding to 5 was introduced into a BTY5 pTY13-HisTAL and was cultured in the presence of 20 g/L of glycerol. The strain corresponding to 5 was used as a resveratrol-producing base strain and indicated by the deeper blue bar. (E) shows the production of resveratrol obtained by transforming 14 synthetic-control sRNAs, selected in Example 2.1, into the base resveratrol-producing strain (BTY5 pTY13-HisTAL pTacCDF-VvSTS-At4CL1m), followed by flask culture. sRNAs corresponding to six strains displayed as deeper red graphs showed more than 2.5-fold increase in resveratrol production compared to the control, and thus were used in combinatorial double knockdown tests. (F) shows the results of combinatorial double knockdown tests using combinations of the previously selected six sRNAs. *P<0.05, **P<0.01, ***P<0.001 (two-tailed Student's t-test).

FIG. 21 shows the results of LC-MS performed to determine the authenticity of resveratrol produced from modified E. coli. (A) shows the LC-MS spectrum of resveratrol produced from modified E. coli, and (B) shows the LC-MS spectrum of a commercially available resveratrol compound.

FIG. 22 shows the naringenin biosynthetic pathway. Here, red X denotes knocked-out genes, and blue X denotes knocked-down genes. Black arrows along with genes written in bold letters denote overexpressed metabolic fluxes. Red dotted lines (along with − signs in circles) indicate transcriptional repression. Black dotted lines (along with + signs in circles) indicate transcriptional activation. bla, beta-lactamase gene; kan^{R}, kanamycin-resistance gene; spc^{R}, spectinomycin-resistance gene; p15A, replication origin; ColE1, replication origin; CDF, replication origin; P_{tac}, tac promoter; P_{BAD}, arabinose-inducible promoter; P_{R}, P_{R} promoter; P_{trc}, trc promoter; rrnB, rrnBT1T2 terminator; T1/TE, terminator. Gly, glycerol; Gly-3P, glycerol 3-phosphate; DHA, dihydroxyacetone; DHAP, dihydroxyacetone phosphate; G3P, glyceraldehyde 3-phosphate; 1,3BPG, 1,3-bisphosphoglycerate; 3PG, 3-phosphoglycerate; 2PG, 2-phosphoglycerate; PEP, phosphoenolpyruvate; PYR, pyruvate; OAA, oxaloacetate; E4P, D-erythrose 4-phosphate; DAHP, 3-deoxy-D-arabinoheptulosonate 7-phosphate; SHIK, shikimate; CHOR, chorismate; PPHN, prephenate; HPP, 4-hydroxyphenylpyruvate; FOR, formate; AcCoA, acetyl-CoA; MalCoA, malonyl-CoA; TYR, L-tyrosine; COU, p-coumaric acid; CouCoA, p-coumaroyl-CoA.

FIG. 23 shows naringenin production in modified E. coli. (A) shows naringenin production in strains containing a pTrcCDF-At4CL1m-AtCHI-PhCHS plasmid. Here, in the case of strains having a p-coumaric acid pathway, the vector was transformed into a BTY5 pTY13-HisTAL strain, and in other cases, the vector was transformed into a BL21(DE3) strain, and then 2 mM p-coumaric acid was added to medium. 20 g/L of glucose or glycerol was added. (B) shows SDS-PAGE analysis of heterologous enzymes. At4CL1m, PhCHS (42.6 kDa), AtCHI (26.6 kDa).

FIG. 24 shows the results of LC-MS performed to determine the authenticity of naringenin produced from modified E. coli. (A) shows the LC-MS spectrum of naringenin produced from modified E. coli, and (B) shows the LC-MS spectrum of a commercially available naringenin compound.

FIG. 25 shows naringenin production in modified E. coli. (A) shows the amount of naringenin produced by transforming 14 synthetic-control sRNAs, selected in Example 2.1, into a base naringenin strain (BTY5 pTY13-HisTAL pTrcCDF-At4CL1m-AtCHI-PhCHS), and then performing flask culture. sRNAs corresponding to three strains displayed as deeper red bars showed an increase in naringenin production of 15% or more compared to the control, and thus were used in combinatorial double knockdown tests. (B) shows the results of combinatorial double knockdown tests using combinations of the previously selected three sRNAs. *P<0.05, **P<0.01 (two-tailed Student's t-test).

### BEST MODE FOR CARRYING OUT THE INVENTION

Unless defined otherwise, all the technical and scientific terms used herein have the same meaning as those generally understood by one of ordinary skill in the art to which the invention pertains. Generally, the nomenclature used herein and the experiment methods, which will be described below, are those well known and commonly employed in the art.

In the present invention, it has been found that the use of a recombinant microorganism introduced with type III polyketide synthase makes it possible to measure the malonyl-CoA concentration in a manner significantly improved in terms of time, cost, convenience and the like, compared to a conventional method of measuring the malonyl-CoA concentration.

Therefore, in one aspect, the present invention is directed to a recombinant microorganism for malonyl-CoA detection in which a type III polyketide synthase-encoding gene is inserted in the genome of the microorganism or in which a recombinant vector containing the type III polyketide synthase-encoding gene is introduced.

In the present invention, the type III polyketide synthase may be:

RppA derived from a microorganism selected from the group consisting of Streptomyces griseus, Streptomyces coelicolor, Streptomyces avermitilis, Saccharopolyspora erythraea, Streptomyces peucetius, and Streptomyces aculeolatus;

PhlD (polyketide synthase) derived from Pseudomonas fluorescens;

DpgA (polyketide synthase) derived from Amycolatopsis mediterranei;

ALS (aloesone synthase) derived from Rheum palmatum; or

PCS (5,7-dihydroxy-2-methylchromone synthase), OKS (octaketide synthase), PKS3 (aloesone synthase), PKS4 (octaketide synthase 2) or PKS5 (octaketide synthase 3), which is derived from Aloe arborescens, but is not limited thereto.

Type III polyketide synthase RppA derived from the above-mentioned six Actinomycetes strains produces flaviolin as a product using malonyl-CoA as a substrate. In addition, other type III polyketide synthases also use malonyl-CoA as a substrate, but PhlD produces phloroglucinol as a product, DpgA produces dihydroxyphenylacetate as a product, PCS produces 5,7-dihydroxy-2-methylchromone as a product, and OKS, PKS4 or PKS5 produces SEK4 and SEK4b as a product. ALS and PKS3 produce aloesone as a product.

The amino acid sequence of each RppA enzyme used in the present invention is as follows:

Streptomyces griseus RppA [SEQ ID NO: 93]:
matlcrpaia
vpehvitmqq tldlaretha ghpqrdlvlr liqntgvqtr hlvqpiektl
ahpgfevrnq vyeaeaktrv pevvrralan aetepseidl ivyvsctgfm
mpsltawiin smgfrpetrq 1piaqlgcaa ggaainrahd fcvaypdsnv
livscefcsl cyqptdigvg sllsnglfgd alsaavvrgq ggtgmrlern
gshlvpdted wisyavrdtg fhfqldkrvp gtmemlapvl ldlvdlhgws
vpnmdffivh aggprilddl chfldlppem frysratlte rgniassvvf
dalarlfddg gaaesaqgli agfgpgitae vavgswakeg lgadvgrdld
eleltagval sg
Streptomyces coelicolor RppA [SEQ ID NO: 94]:
matlcrpsvs
vpehvitmee tlelarrrht dhpqlplalr lientgvrtr hivqpiedtl
ehpgfedrnk vyereaksrv paviqraldd aellatdidv iiyvsctgfm
mpsltawlin emgfdsttrq ipiaqlgcaa ggaainrahd fctaypeana
livacefcsl cyqptdlgvg sllcnglfgd giaaavvrgr ggtgvrlern
gsylipkted wimydvkatg fhflldkrvp atmeplapal kelagehgwd
asdldfyivh aggprilddl stflevdpha frfsratlte ygniasavvl
dalrrlfdeg gveegargll agfgpgitae mslgcwqtad vrrgirqdvt
rtaargvsrr vrqa
Streptomyces avermitilis RppA [SEQ ID NO: 95]:
matlckpavs vpehvitmee tlelarsrhp dhpqlplalr lientgvhtr
hivqpieet1 khpgfeernh vyeaeakary pavvqralde aellttdidv
iiyvsctgfm mpsltaylin smdfssdtrq ipiaqlgcaa ggsainrahd
fctaypqana livacefcsl cyqptdlgvg sllsnglfgd giaaaavrgk
ggtgitlern asylipktde wisydvratg fhflldkrvp gtmeplapal
qelasqhgwd asdldfyiih aggprilddl skflrvppea frfsratlte
ygniasavvl dalrrlfdeg gaehaargml agfgpgitae mslgrwhrtd ea
Saccharopolyspora erythraea RppA [SEQ ID NO: 96]:
mavlctpava vpehvitvee tldlarrvha dhpq1p1v1r lisntgvrer
hlirpiedt1 ehpgfevrnr iyeeqakqrv pavvrealds aelgpedidl
ivyvsctgfm mpsltawlin smgfrmstrq 1piaqlgcaa ggaainrahd
fctaypdana livscefcsl cyqptdddig sllsnglfgd avgaavvrgh
ggtgvrlern assmipeted wisyavkatg fhfqldkrvp ktmeplapal
ralaedhrwd vagldfyvih aggprilddl tkflgvpsea frhsratlaq
ygniasavvl dalrriieeg rlesgargmi agfgpgitae msvgtwvphd
vllhgehstt sapggnr
Streptomyces peucetius RppA [SEQ ID NO: 97]:
mrvpvavddl
vapstmgerh tvidrgtsva avhtalpphr yaqsdlteli adlclepgad
rallrrlhts agvrtrhlal pieqyaglgd fgqanaawlt vglalaeeal
sgaldaaglt aadidllvct sitgvaapsl darlavrmgm radvkrvpvf
glgcvggaag lgrlhdyllg hpddtavlls velcsltlqr dgslanlvag
alfgdgaaav varggdagrr gagwpmvaat rghlypdteh llgwrigasg
frvvvdagip dvvrthlggd lrnflathgl vpddigtwic hpggpkvlaa
vgdalelpdg aldsswrsla gvgnlssasv lrvledvatr crpdpgtwgv
llamgpgfca efvllrw
Streptomyces aculeolatus RppA [SEQ ID NO: 98]:
mprlckpavs apeytitmee tlefakqaha gkpqlplalr lirntgvlkr
hivqpiektl ghpglternl iyeaeskkmc ppvieealqn admtardida
iiyvsctgfl mpsltawlin kmgfrsdtrq ipiaqlgcaa ggaavnrahd
fclahpgsnv livacelcsl cyqptaddig sllsdglfgd avaaavvrgn
ggvgievern asylipntee wisysvrdtg fhfqldrrvp gtmeplapvl
refakdhswd agkldfyivh aggprilddl arfldvdrqv frhswstlte
ygniasavvf daarrlfeeg sakpdatgmi agfgpgitae malgtwgtdg tgtsn
The amino acid sequences of other type III polyketide
synthases used in the present invention are as follows:
Pseudomonas fluorescens type III polyketide synthase
(PhlD)[SEQ ID NO: 99]:
MSTLCKPSLL FPHYKITQQQ MIDHLEQLHD DHPRMALAKR MIQNTQVNER
YLVLPIDELA VHTGFTHRSI VYEREARRMS SIAARQAIEN AGLTTDDIRM
VAVTSCTGFM MPSLTAHLIN DLGLRTSTVQ LPIAQLGCVA GAAAINRAND
FASLSPDNHA LIVSLEFSSL CYQPQDTKLH AFISAALFGD AVSACVMRAD
DKAPGFKIAK TGSYFLPDSE HYIKYDVKDS GFHFTLDKAV MNSIKDVAPM
MEELNFETFN QHCAQNDFFI FHTGGRKILD ELVLQLDLEP GRVAQSRDSL
SEAGNIASVV VFDVLKRQFD SGPANGATGM LAAFGPGFTA EMAVGKWVA
Amycolatopsis orientalis type III polyketide synthase
(DpgA)[SEQ ID NO: 100]:
MDVSMTTGIE LTEELSVLNG LTEITRFAGV GTAVSETSYS QTELLDILDV
EDPKIRSVFL NSAIDRRFLT LPPENPGGGR LAEPQGDLLD KHKKIAVDMG
CRALEACLKS AGATLSDLRH LCCVTSTGFL TPGLSALIIR EMGIDPHCSR
SDIVGMGCNA GLNALNVVSG WSAAHPGELG VVLCSEACSA AYALDGTMRT
AVVNSLFGDG SAALAVISGD GRVAGPRVLK FASYIITDAV DAMRYDWDRD
QDRFSFFLDP QIPYVVGAHA EIVVDRLLSG TGLRRSDIGH WLVHSGGKKV
VDAVVVNLGL SRHDVRHTTG VLRDYGNLSS GSFLFSYERL SEEDVTRPGD
YGVLMTMGPG STIEMALIQW
Rheum palmatum type III polyketide synthase (aloesone
synthase, ALS) [SEQ ID NO: 101]:
madvlqeirn sqkasgpatv laigtahppt cypqadypdf yfrvcksehm
tklkkkmqfi cdrsgirqrf mfhteenlgk npgmctfdgp slnarqdmli
mevpklgaea aekaikewgq dksrithlif ctttsndmpg adyqfatlfg
lnpgvsrtmv yqqgcfaggt vlrlvkdiae nnkgarvlvv cseivafafr
gphedhidsl igqllfgdga aalvvgtdid esverpifqi msatqatipn
slhtmalhlt eagltfhlsk evpkvvsdnm eelmleafkp lgitdwnsif
wqvhpggrai ldkieeklel tkdkmrdsry ilseygnlts acvlfvmdem
rkrsfregkq ttgdgyewgv aiglgpgltv etvvlrsvpi p
Aloe arborescens type III polyketide synthase (5,7-
dihydroxy-2-methylchromone synthase, PCS)[SEQ ID NO: 102]:
MSSLSNSLPL MEDVQGIRKA QKADGTATVM AIGTAHPPHI FPQDTYADVY
FRATNSEHKV ELKKKFDHIC KKTMIGKRYF NYDEEFLKKY PNITSYDEPS
LNDRQDICVP GVPALGTEAA VKAIEEWGRP KSEITHLVFC TSCGVDMPSA
DFQCAKLLGL HANVNKYCIY MQGCYAGGTV MRYAKDLAEN NRGARVLVVC
AELTIMMLRA PNETHLDNAI GISLFGDGAA ALIIGSDPII GVEKPMFEIV
CTKQTVIPNT EDVIHLHLRE TGMMFYLSKG SPMTISNNVE ACLIDVFKSV
GITPPEDWNS LFWIPHPGGR AILDQVEAKL KLRPEKFRAA RTVLWDYGNM
VSASVGYILD EMRRKSAAKG LETYGEGLEW GVLLGFGPGI TVETILLHSL PLM
Aloe arborescens type III polyketide synthase
(octaketide synthase, OKS)[SEQ ID NO: 103]:
MSSLSNASHL MEDVQGIRKA QRADGTATVM AIGTAHPPHI FPQDTYADFY
FRATNSEHKV ELKKKFDRIC KKTMIGKRYF NYDEEFLKKY PNITSFDEPS
LNDRQDICVP GVPALGAEAA VKAIAEWGRP KSEITHLVFC TSCGVDMPSA
DFQCAKLLGL RTNVNKYCVY MQGCYAGGTV MRYAKDLAEN NRGARVLVVC
AELTIIGLRG PNESHLDNAI GNSLFGDGAA ALIVGSDPII GVEKPMFEIV
CAKQTVIPNS EDVIHLHMRE AGLMFYMSKD SPETISNNVE ACLVDVFKSV
GMTPPEDWNS LFWIPHPGGR AILDQVEAKL KLRPEKFRAT RTVLWDCGNM
VSACVLYILD EMRRKSADEG LETYGEGLEW GVLLGFGPGM TVETILLHSL PLM
Aloe arborescens type III polyketide synthase (aloesone
synthase, PKS3)[SEQ ID NO: 104]:
mgslsdstpl mkdvqgirka qkadgtatvm aigtahpphi isqdsyadfy
frvtnsehkv elkkkfdric kktmigkryf nfdeeflkky pnitsfdkps
lndrhdicip gvpalgaeaa vkaieewgrp kseithlvfc tsggvdmpsa
dfqcakllgl rtnvnkyciy mqgcyaggtv mryakdlaen nrgarvlmvc
aeltiialrg pndshidnai gnslfgdgaa alivgsdpii gvekpmfeiv
cakqtvipns eevihlhlre sglmfymtkd saatisnnie aclvdvfksv
gmtppedwns lfwiphpggr aildqveakl klrpekfsat rtvlwdygnm
isacvlyild emrrksaaeg letygeglew gvllgfgpgm tietillhsl ppv
Aloe arborescens type III polyketide synthase
(octaketide synthase 2, PKS4)[SEQ ID NO: 105]:
MGSLSNYSPV MEDVQAIRKA QKADGTATVM AIGTAHPPHI FPQDTYADFY
FRATNSEHKV ELKKKFDRIC KKTMIGKRYF NYDEEFLKKY PNITSFDEPS
LNDRQDICVP GVPALGAEAA VKAIAEWGRP KSEITHLVFC TSCGVDMPSA
DFQCAKLLGL RTNVNKYCVY MQGCYAGGTV MRYAKDLAEN NRGARVLVVC
AELTIIGLRG PNESHLDNAI GNSLFGDGAA ALIVGSDPII GVERPMFEIV
CAKQTVIPNS EDVIHLHMRE AGLMFYMSKD SPETISNNVE ACLVDVFKSV
GMTPPEDWNS LFWIPHPGGR AILDQVEARL KLRPEKFGAT RTVLWDCGNM
VSACVLYILD EMRRKSVADG LATYGEGLEW GVLLGFGPGM TVETILLHSL PPV
Aloe arborescens type III polyketide synthase
(octaketide synthase 3, PKS5)[SEQ ID NO: 106]:
MGSIAESSPL MSRENVEGIR KAQRAEGTAT VMAIGTAHPP HIFPQDTYAD
FYFRATNSEH KVELKKKFDR ICKKTMIGKR YFNYDEEFLK KYPNITSFDE
PSLNDRQDIC VPGVPALGKE AALKAIEEWG QPLSKITHLV FCTSCGVDMP
SADFQLAKLL GLNTNVNKYC VYMQGCYAGG TVLRYAKDLA ENNRGSRVLV
VCAELTIIGL RGPNESHLDN AIGNSLFGDG AAALIVGADP IVGIEKPIFE
IVCAKQTVIP DSEDVIHLHL REAGLMFYMS KDSPETISNN VEGCLVDIFK
SVGMTPPADW NSLFWIPHPG GRAILDEVEA RLKLRPEKFR ATRHVLWEYG
NMVSACVLYI LDEMRNKSAA DGLGTYGEGL EWGVLLGFGP GMTVETILLH SLPPV

The amino acid sequence of the above-described type III polyketide synthase illustrates some feasible enzymes for constructing the recombinant microorganism for malonyl-CoA detection according to the present invention. The present invention has a technical feature in that malonyl-CoA detection is possible by introduction of type III polyketide synthase. Thus, it will be obvious to those skilled in the art that the present invention makes it possible to construct recombinant microorganisms introduced with type III polyketide synthases other than the above-described type III polyketide synthase.

In the present invention, the recombinant microorganism may be characterized in that an gene encoding the type III polyketide synthase is operably linked to a promoter selected from the group consisting of tac, trc, T7, BAD, λPR an Anderson synthetic promoter, but is not limited thereto.

In the present invention, the recombinant microorganism may be selected from the strain group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhodobacter, Zymomonas, Bacillus, Staphylococcus, Lactococcus, Streptococcus, Lactobacillus, Clostridium, Corynebacterium, Streptomyces, Bifidobacterium, Cyanobacterium, and Cyclobacterium, but the microorganism to which the present invention is applied is not limited thereto. Preferably, the recombinant microorganism of the present invention may be constructed by introducing a recombinant vector into a microorganism selected from the group consisting of E. coli, Pseudomonas sp., Corynebacterium sp., and Rhodococcus sp., but is not limited thereto. As E. coli, one selected from among the E. coli strains shown in Table 2 below is preferably used, but those skilled in the art will appreciate that other E. coli strains expected to exhibit similar effects may also be used. The recombinant microorganism of the present invention may be constructed by using Pseudomonas putida as Pseudomonas sp., Corynebacterium glutamicum as Corynebacterium sp., and Rhodococcus opacus as Rhodococcus sp., but this is an example of a representative microorganism, and the microorganism to which the present invention is applied is not limited thereto.

In the meantime, in the present invention, when the recombinant microorganism for malonyl-CoA detection is used in which a type III polyketide synthase-encoding gene is inserted in the genome of the microorganism or in which a recombinant vector containing the type III polyketide synthase-encoding gene is introduced, the increase and decrease in the production of the malony-CoA could be easily confirmed through the concentration-dependent color development from the malony-CoA, and a malonyl-CoA production-inducing substance may be easily screened.

Therefore, in another aspect, the present invention is directed to a method for screening a malonyl-CoA production-inducing substance, comprising the steps of:

(a) culturing the recombinant microorganism;

(b) adding a candidate substance to the recombinant microorganism;

(c) comparing the color of a culture supernatant of the recombinant microorganism after addition of the candidate substance with the color of a culture supernatant of the recombinant microorganism without addition of the candidate substance; and

(d) selecting the candidate substance as the malonyl-CoA production-inducing substance, when the culture supernatant of the recombinant microorganism after addition of the candidate substance shows a deeper red color than the culture supernatant of the recombinant microorganism without addition of the candidate substance.

In the present invention, step (c) may comprise clearly observing a change in the color by the naked eyes, or measuring absorbance and quantitatively expressing a change in the absorbance.

Thus, the comparison between the colors in step (c) may be made by the naked eyes.

In addition, the comparison between the colors in step (c) may be made by measuring absorbance, and step (d) may comprises selecting the candidate substance as the malonyl-CoA production-inducing substance, when the absorbance measured after addition of the candidate substance is higher than that measured without addition of the candidate substance.

In the present invention, the absorbance (OD value) at 280 to 450 nm, preferably 300 to 340 nm, may be measured, and the OD value increases as the malonyl-CoA concentration increases.

The malonyl-CoA production-inducing substance can induce the production of the malonyl-CoA by a mechanism that regulates expression of the gene involved in increased malonyl-CoA production.

Therefore, in still another aspect, the present invention is directed to a method for screening a gene involved in increase of malonyl-CoA production, the method comprising the steps of:

(a) introducing a gene regulation library, which changes gene expression in a recombinant microorganism, into the above-described recombinant microorganism, thereby constructing a recombinant microorganism library in which gene expression in the recombinant microorganism is changed;

(b) culturing the constructed recombinant microorganism library, measuring the absorbance of the culture supernatant of the recombinant microorganism library, culturing the recombinant microorganism before introduction of the gene regulation library, and comparing the color of the culture supernatant of the recombinant microorganism library with the color of the culture supernatant of the recombinant microorganism before introduction of the gene regulation library; and

(c) selecting the gene introduced in the recombinant microorganism library, when the culture supernatant of the recombinant microorganism library shows a deeper red color than the culture supernatant of the recombinant microorganism before introduction of the gene regulation library.

In the present invention, step (b) may comprise clearly observing a change in the color by the naked eyes, or measuring absorbance and quantitatively expressing a change in the absorbance.

Thus, the comparison of the color in step (c) may be made by the naked eyes.

In addition, the comparison between the colors in step (b) is made by measuring absorbance, and step (c) may comprise selecting the gene introduced in the recombinant microorganism library as a gene involved in increase of malonyl-CoA production, when the absorbance of the culture supernatant of the recombinant microorganism library is higher than that of the culture supernatant of the recombinant microorganism before introduction of the gene regulation library.

In the present invention, the absorbance (OD value) at 280 to 450 nm, preferably 300 to 340 nm, may be measured, and the OD value increases as the malonyl-CoA concentration increases.

In the present invention, the gene regulation library may be a library selected from an sRNA library, a genomic library, a cDNA library, a gRNA library, and an oligonucleotide library for construction of knockout or mutant strains, but is not limited thereto.

Specifically, to achieve the object of the present invention, the gene regulation library may be suitably selected from among the following:

an sRNA library, or an oligonucleotide library for construction of knockout or mutant strains for inhibiting endogenous gene expression in the recombinant microorganism;

a genomic library or a cDNA library which is a library for endogenous or exogenous gene overexpression; and

a gRNA (guide RNA which is used in CRISPR (Clustered Regularly Interspaced Short Palindromic Repeats)-Cas9 technology) library which is a library enabling both gene expression inhibition and gene overexpression.

That is, the addition of the synthetic-control sRNA library described in Example 2 below is an example to describe the present invention in detail, and presents the method of using the same, and it is possible to introduce any other libraries known to exhibit similar functions.

Meanwhile, when the gene screened by the method is knocked down in a microorganism, malonyl-CoA production in the microorganism can be increased. Therefore, in yet another aspect, the present invention is directed to a method for producing a recombinant microorganism having increased malonyl-CoA producing ability, the method comprising regulating expression of the gene, screened by the method, in a microorganism having malonyl-CoA producing ability.

The microorganism having malonyl-CoA producing ability means a microorganism inherently having malonyl-CoA producing ability or externally introduced with malonyl-CoA producing ability.

In the present invention, the screened gene may be one or more genes selected from the group consisting of: fabF (3-oxoacyl-[acyl-carrier-protein] synthase II);

yfcY (beta-ketoacyl-CoA thiolase);

xapR (transcriptional activator of xapAB);

cytR (transcriptional repressor for deo operon, udp, cdd, tsx, nupC and nupG);

fabH (3-oxoacyl-[acyl-carrier-protein] synthase III);

mqo (malate dehydrogenase);

yfiD (pyruvate formate lyase subunit);

fmt (10-formyltetrahydrofolate:L-methionyl-tRNA(fMet)N-formyltransferase);

pyrF (orotidine-5′-phosphate decarboxylase);

araA (L-arabinose isomerase);

fadR (negative regulator for fad regulon and positive regulator of fabA);

pabA (aminodeoxychorismate synthase, subunit II);

purB (adenylosuccinate lyase); and

hycI (protease involved in processing C-terminal end of HycE),

and expression of the gene may be reduced, but is not limited thereto.

In the present invention, fabF encodes 3-oxoacyl-[acyl-carrier-protein] synthase II, yfcY encodes beta-ketoacyl-CoA thiolase, xapR encodes transcriptional activator of xapAB, cytR encodes transcriptional repressor for deo operon, udp, cdd, tsx, nupC and nupG, fabH encodes 3-oxoacyl-[acyl-carrier-protein] synthase III, mqo encodes malate dehydrogenase, yfiD encodes pyruvate formate lyase subunit, fmt encodes 10-formyltetrahydrofolate:L-methionyl-tRNA(fMet)N-formyltransferase, pyrF encodes orotidine-5′-phosphate decarboxylase, araA encodes L-arabinose isomerase, fadR encodes negative regulator for fad regulon and positive regulator of fabA, pabA encodes aminodeoxychorismate synthase, subunit II, purB encodes adenylosuccinate lyase, and hycI encodes protease involved in processing C-terminal end of HycE, respectively.

The amino acid sequence of each of enzymes, which are screened and used in the present invention, is as follows, but may have a substitution, deletion or addition of one or more amino acids in the amino acid sequences, and in this case, the resulting mutant amino acid sequence may exhibit a function equal to or better than that in the present invention. For example, it will be obvious that if the structural configuration of the mutant amino acid sequence is identical or similar to the wild-type amino acid sequence, and thus exhibits the same or higher effect on a substrate, the mutant amino acid sequence also falls within the scope of the present invention. In the same context, it will be obvious that besides enzymes used in the present invention, other microorganism-derived enzymes having an identical or similar function can be properly applied to the present invention, and the application of a corresponding enzyme within a range that can be easily applied by a person of ordinary skill in the art falls within the scope of the present invention.

FabF[SEQ ID NO: 107]:
MSKRRVVVTGLGMLSPVGNTVESTWKALLAGQSGISLIDHFDTSAYATKFAGLVKD
FNCEDIISRKEQRKMDAFIQYGIVAGVQAMQDSGLEITEENATRIGAAIGSGIGGLGLIEEN
HTSLMNGGPRKISPFFVPSTIVNMVAGHLTIMYGLRGPSISIATACTSGVHNIGHAARIIAY
GDADVMVAGGAEKASTPLGVGGFGAARALSTRNDNPQAASRPWDKERDGFVLGDGAGMLVLE
EYEHAKKRGAKIYAELVGFGMSSDAYHMTSPPENGAGAALAMANALRDAGIEASQIGYVNAH
GTSTPAGDKAEAQAVKTIFGEAASRVLVSSTKSMTGHLLGAAGAVESIYSILALRDQAVPPT
INLDNPDEGCDLDFVPHEARQVSGMEYTLCNSFGFGGTNGSLIFKKI
YfcY[SEQ ID NO: 108]:
MGQVLPLVTRQGDRIAIVSGLRTPFARQATAFHGIPAVDLGKMVVGELLARSEIPA
EVIEQLVFGQVVQMPEAPNIAREIVLGTGMNVHTDAYSVSRACATSFQAVANVAESLMAGTI
RAGIAGGADSSSVLPIGVSKKLARVLVDVNKARTMSQRLKLFSRLRLRDLMPVPPAVAEYST
GLRMGDTAEQMAKTYGITREQQDALAHRSHQRAAQAWSDGKLKEEVMTAFIPPYKQPLVEDN
NIRGNSSLADYAKLRPAFDRKHGTVTAANSTPLTDGAAAVILMTESRAKELGLVPLGYLRSY
AFTAIDVWQDMLLGPAWSTPLALERAGLTMSDLTLIDMHEAFAAQTLANIQLLGSERFAREA
LGRAHATGEVDDSKFNVLGGSIAYGHPFAATGARMITQTLHELRRRGGGFGLVTACAAGGLG
AAMVLEAE
XapR[SEQ ID NO: 109]:
MERVYRTDLKLLRYFLAVAEELHFGRAAARLNMSQPPLSIHIKELENQLGTQLFIR
HSRSVVLTHAGKILMEESRRLLVNANNVLARIEQIGRGEAGRIELGVVGTAMWGRMRPVMRR
FLRENPNVDVLFREKMPAMQMALLERRELDAGIWRMATEPPTGFTSLRLHESAFLVAMPEEH
HLSSFSTVPLEALRDEYFVTMPPVYTDWDFLQRVCQQVGFSPVVIREVNEPQTVLAMVSMGI
GITLIADSYAQMNWPGVIFRPLKQRIPADLYIVYETQQVTPAMVKLLAALTQ
CytR[SEQ ID NO: 110]:
MKAKKQETAATMKDVALKAKVSTATVSRALMNPDKVSQATRNRVEKAAREVGYLPQ
PMGRNVKRNESRTILVIVPDICDPFFSEIIRGIEVTAANHGYLVLIGDCAHQNQQEKTFIDL
IITKQIDGMLLLGSRLPFDASIEEQRNLPPMVMANEFAPELELPTVHIDNLTAAFDAVNYLY
EQGHKRIGCIAGPEEMPLCHYRLQGYVQALRRCGIMVDPQYIARGDFTFEAGSKAMQQLLDL
PQPPTAVFCHSDVMALGALSQAKRQGLKVPEDLSIIGFDNIDLTQFCDPPLTTIAQPRYEIG
REAMLLLLDQMQGQHVGSGSRLMDCELIIRGSTRALP
FabH[SEQ ID NO: 111]:
MYTKIIGTGSYLPEQVRTNADLEKMVDTSDEWIVTRTGIRERHIAAPNETVSTMGF
EAATRAIEMAGIEKDQIGLIVVATTSATHAFPSAACQIQSMLGIKGCPAFDVAAACAGFTYA
LSVADQYVKSGAVKYALVVGSDVLARTCDPTDRGTIIIFGDGAGAAVLAASEEPGIISTHLH
ADGSYGELLTLPNADRVNPENSIHLTMAGNEVFKVAVTELAHIVDETLAANNLDRSQLDWLV
PHQANLRIISATAKKLGMSMDNVVVTLDRHGNTSAASVPCALDEAVRDGRIKPGQLVLLEAF
GGGFTWGSALVRF
Mqo[SEQ ID NO: 112]:
MKKVTAMLFSMAVGLNAVSMAAKAKASEEQETDVLLIGGGIMSATLGTYLRELEPE
WSMTMVERLEGVAQESSNGWNNAGTGHSALMELNYTPQNADGSISIEKAVAINEAFQISRQF
WAHQVERGVLRTPRSFINTVPHMSFVWGEDNVNFLRARYAALQQSSLFRGMRYSEDHAQIKE
WAPLVMEGRDPQQKVAATRTEIGTDVNYGEITRQLIASLQKKSNFSLQLSSEVRALKRNDDN
TWTVTVADLKNGTAQNIRAKFVFIGAGGAALKLLQESGIPEAKDYAGFPVGGQFLVSENPDV
VNHHLAKVYGKASVGAPPMSVPHIDTRVLDGKRVVLFGPFATFSTKFLKNGSLWDLMSSTTT
SNVMPMMHVGLDNFDLVKYLVSQVMLSEEDRFEALKEYYPQAKKEDWRLWQAGQRVQIIKRD
AEKGGVLRLGTEVVSDQQGTIAALLGASPGASTAAPIMLNLLEKVFGDRVSSPQWQATLKAI
VPSYGRKLNGDVAATERELQYTSEVLGLNYDKPQAADSTPKPQLKPQPVQKEVADIAL
YfiD[SEQ ID NO: 113]:
MITGIQITKAANDDLLNSFWLLDSEKGEARCIVAKAGYAEDEVVAVSKLGDIEYRE
VPVEVKPEVRVEGGQHLNVNVLRRETLEDAVKHPEKYPQLTIRVSGYAVRFNSLTPEQQRDV
IARTFTESL
Fmt[SEQ ID NO: 114]:
MSESLRIIFAGTPDFAARHLDALLSSGHNVVGVFTQPDRPAGRGKKLMPSPVKVLA
EEKGLPVFQPVSLRPQENQQLVAELQADVMVVVAYGLILPKAVLEMPRLGCINVHGSLLPRW
RGAAPIQRSLWAGDAETGVTIMQMDVGLDTGDMLYKLSCPITAEDTSGTLYDKLAELGPQGL
ITTLKQLADGTAKPEVQDETLVTYAEKLSKEEARIDWSLSAAQLERCIRAFNPWPMSWLEIE
GQPVKVWKASVIDTATNAAPGTILEANKQGIQVATGDGILNLLSLQPAGKKAMSAQDLLNSR
REWFVPGNRLV
PyrF [SEQ ID NO: 115]:
MTLTASSSSRAVTNSPVVVALDYHNRDDALAFVDKIDPRDCRLKVGKEMFTLFGPQ
FVRELQQRGFDIFLDLKFHDIPNTAAHAVAAAADLGVWMVNVHASGGARMMTAAREALVPFG
KDAPLLIAVTVLTSMEASDLVDLGMTLSPADYAERLAALTQKCGLDGVVCSAQEAVRFKQVF
GQEFKLVTPGIRPQGSEAGDQRRIMTPEQALSAGVDYMVIGRPVTQSVDPAQTLKAINASLQ
RSA
AraA [SEQ ID NO: 116]:
MTIFDNYEVWFVIGSQHLYGPETLRQVTQHAEHVVNALNTEAKLPCKLVLKPLGTT
PDEITAICRDANYDDRCAGLVVWLHTFSPAKMWINGLTMLNKPLLQFHTQFNAALPWDSIDM
DFMNLNQTAHGGREFGFIGARMRQQHAVVTGHWQDKQAHERIGSWMRQAVSKQDTRHLKVCR
FGDNMREVAVTDGDKVAAQIKFGFSVNTWAVGDLVQVVNSISDGDVNALVDEYESCYTMTPA
TQIHGKKRQNVLEAARIELGMKRFLEQGGFHAFTTTFEDLHGLKQLPGLAVQRLMQQGYGFA
GEGDWKTAALLRIMKVMSTGLQGGTSFMEDYTYHFEKGNDLVLGSHMLEVCPSIAAEEKPIL
DVQHLGIGGKDDPARLIFNTQTGPAIVASLIDLGDRYRLLVNCIDTVKTPHSLPKLPVANAL
WKAQPDLPTASEAWILAGGAHHTVFSHALNLNDMRQFAEMHDIEITVIDNDTRLPAFKDALR
WNEVYYGFRR
ArgC[SEQ ID NO: 117]:
MLNTLIVGASGYAGAELVTYVNRHPHMNITALTVSAQSNDAGKLISDLHPQLKGIV
DLPLQPMSDISEFSPGVDVVFLATAHEVSHDLAPQFLEAGCVVFDLSGAFRVNDATFYEKYY
GFTHQYPELLEQAAYGLAEWCGNKLKEANLIAVPGCYPTAAQLALKPLIDADLLDLNQWPVI
NATSGVSGAGRKAAISNSFCEVSLQPYGVFTHRHQPEIATHLGADVIFTPHLGNFPRGILET
ITCRLKSGVTQAQVAQVLQQAYAHKPLVRLYDKGVPALKNVVGLPFCDIGFAVQGEHLIIVA
TEDNLLKGAAAQAVQCANIRFGYAETQSLI
FadR[SEQ ID NO: 118]:
MVIKAQSPAGFAEEYIIESIWNNRFPPGTILPAERELSELIGVIRTTLREVLQRLA
RDGWLTIQHGKPTKVNNFWETSGLNILETLARLDHESVPQLIDNLLSVRTNISTIFIRTAFR
QHPDKAQEVLATANEVADHADAFAELDYNIFRGLAFASGNPIYGLILNGMKGLYTRIGRHYF
ANPEARSLALGFYHKLSALCSEGAHDQVYETVRRYGHESGEIWHRMQKNLPGDLAIQGR
NudD[SEQ ID NO: 119]:
MFLRQEDFATVVRSTPLVSLDFIVENSRGEFLLGKRTNRPAQGYWFVPGGRVQKDE
TLEAAFERLTMAELGLRLPITAGQFYGVWQHFYDDNFSGTDFTTHYVVLGFRFRVSEEELLL
PDEQHDDYRWLTSDALLASDNVHANSRAYFLAEKRTGVPGL
PabA[SEQ ID NO: 120]:
MILLIDNYDSFTWNLYQYFCELGADVLVKRNDALTLADIDALKPQKIVISPGPCTP
DEAGISLDVIRHYAGRLPILGVCLGHQAMAQAFGGKVVRAAKVMHGKTSPITHNGEGVFRGL
ANPLTVTRYHSLVVEPDSLPACFDVTAWSETREIMGIRHRQWDLEGVQFHPESILSEQGHQL
LANFLHR
PurB[SEQ ID NO: 121]:
MELSSLTAVSPVDGRYGDKVSALRGIFSEYGLLKFRVQVEVRWLQKLAAHAAIKEV
PAFAADAIGYLDAIVASFSEEDAARIKTIERTTNHDVKAVEYFLKEKVAEIPELHAVSEFIH
FACTSEDINNLSHALMLKTARDEVILPYWRQLIDGIKDLAVQYRDIPLLSRTHGQPATPSTI
GKEMANVAYRMERQYRQLNQVEILGKINGAVGNYNAHIAAYPEVDWHQFSEEFVTSLGIQWN
PYTTQIEPHDYIAELFDCVARFNTILIDFDRDVWGYIALNHFKQKTIAGEIGSSTMPHKVNP
IDFENSEGNLGLSNAVLQHLASKLPVSRWQRDLTDSTVLRNLGVGIGYALIAYQSTLKGVSK
LEVNRDHLLDELDHNWEVLAEPIQTVMRRYGIEKPYEKLKELTRGKRVDAEGMKQFIDGLAL
PEEEKARLKAMTPANYIGRAITMVDELK
HycI[SEQ ID NO: 122]:
MTDVLLCVGNSMMGDDGAGPLLAEKCAAAPKGNWVVIDGGSAPENDIVAIRELRPT
RLLIVDATDMGLNPGEIRIIDPDDIAEMFMMTTHNMPLNYLIDQLKEDIGEVIFLGIQPDI
VGFYYPMTQPIKDAVETVYQRLEGWEGNGGFAQLAVEEE

In the present invention, the microorganism may be selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhodobacter, Zymomonas, Bacillus, Staphylococcus, Lactococcus, Streptococcus, Lactobacillus, Clostridium, Corynebacterium, Streptomyces, Bifidobacterium, Cyanobacterium, and Cyclobacterium, but the microorganism to which the present invention can be applied is not limited thereto. Preferably, the microorganism of the present invention may be constructed by introducing a recombinant vector into a microorganism selected from the group consisting of E. coli, Pseudomonas sp., Corynebacterium sp., and Rhodococcus sp., but is not limited thereto. As E. coli, one selected from among the E. coli strains shown in Table 2 below is preferably used, but those skilled in the art will appreciate that other E. coli strains expected to exhibit similar effects may also be used. The recombinant microorganism of the present invention may be constructed by using Pseudomonas putida as Pseudomonas sp., Corynebacterium glutamicum as Corynebacterium sp., and Rhodococcus opacus as Rhodococcus sp., but this is an example of a representative microorganism, and the microorganism to which the present invention is applied is not limited thereto.

In addition, the use of the screened gene can produce a useful substance using malonyl-CoA as a substrate or an intermediate in high yield by only a very simple genetic manipulation.

Therefore, in a first further aspect, the present invention is directed to a recombinant microorganism having increased malonyl-CoA producing ability wherein expression of one or more genes selected from the group consisting of the following genes:

fabF (3-oxoacyl-[acyl-carrier-protein] synthase II);

yfcY (beta-ketoacyl-CoA thiolase);

xapR (transcriptional activator of xapAB);

cytR (transcriptional repressor for deo operon, udp, cdd, tsx, nupC and nupG);

fabH (3-oxoacyl-[acyl-carrier-protein] synthase III);

mqo (malate dehydrogenase);

yfiD (pyruvate formate lyase subunit);

fmt (10-formyltetrahydrofolate:L-methionyl-tRNA(fMet)N-formyltransferase);

pyrF (orotidine-5′-phosphate decarboxylase);

araA (L-arabinose isomerase);

fadR (negative regulator for fad regulon and positive regulator of fabA);

pabA (aminodeoxychorismate synthase, subunit II);

purB (adenylosuccinate lyase); and

hycI (protease involved in processing C-terminal end of HycE), in a microorganism having malonyl-CoA producing ability is decreased compared to that in a wild-type microorganism.

The microorganism having malonyl-CoA producing ability means a microorganism inherently having malonyl-CoA producing ability or externally introduced with malonyl-CoA producing ability.

The present invention is also directed to a method of producing a useful substance using malonyl-CoA as a substrate or an intermediate, the method comprising the steps of:

(a) constructing a recombinant microorganism in which a gene involved in production of the useful substance is additionally introduced, or expression of the gene is increased, or a gene involved in production of the useful substance is additionally deleted, or expression of the gene is decreased;

(b) culturing the constructed microorganism; and

(c) recovering the useful substance from the cultured microorganism.

In the present invention, the useful substance may be one or more useful substances selected from among:

a polyketide compound composed of actinorhodin, doxorubicin, daunorubicin, oxytetracycline, rapamycin, lovastatin, SEK4, SEK4b, SEK34, SEK15, SEK26, FK506, DMAC, aklavinone, aklanonic acid, epsilon-rhodomycinone, aloesin, aloenin, barbaloin, 5,7-dihydroxy-2-methylchromone, erythromycin, rifamycin, avermectin, geldanamycin, ivermectin, doxycycline, anthramycin, penicillic acid, calicheamicin, epothilone, tetracenomycin, frenolicin, triacetic acid lactone, 6-methylsalicylic acid, and aloesone;

a phenylpropanoid compound composed of pinocembrin, dihydrokaempferol, eriodictyol, dihydroquercetin, daidzein, genistein, apigenin, luteolin, kaempferol, quercetin, catechin, pelargonidin, cyanidin, afzelechin, myricetin, fisetin, galangin, hesperetin, tangeritin, delphinidin, epicatechin, chrysin, resveratrol, and naringenin;

a biofuel group composed of pentane, hexane, heptane, octane, nonane, decane, undecane, dodecane, tridecane, tetradecane, pentadecane, hexadecane, heptadecane, octadecane, nonadecane, icosane, pentanol, hexanol, heptanol, octanol, nonanol, decanol, undecanol, dodecanol, tridecanol, tetradecanol, pentadecanol, hexadecanol, heptadecanol, octadecanol, nonadecanol, icosanol, methyl caprate, methyl laurate, methyl myristate, methyl palmitate, methyl palmitoleate, methyl stearate, methyl oleate, methyl linoleate, methyl linolenate, methyl arachidate, methyl paullinate, methyl erucate, ethyl caprate, ethyl laurate, ethyl myristate, ethyl palmitate, ethyl palmitoleate, ethyl stearate, ethyl oleate, ethyl linoleate, ethyl linolenate, ethyl arachidate, ethyl paullinate, and methyl erucate;

a lipid compound composed of ceramide, palmitate, and sphingosine; and

3-hydroxypropionic acid, but is not limited thereto. In other words, the biosensor (i.e., recombinant microorganism) of the present invention can be applied to high-valued products such as other any malonyl-CoA-derived natural substances, compounds, biofuels, and the like, besides the above-described useful substances.

The present invention is also directed to a recombinant microorganism for producing 6-methylsalicylic acid in which genes that encode 6-methylsalicylic acid synthase (6MSAS) and 4′-phosphopantetheinyl transferase (Sfp) are additionally introduced or expression of the genes is increased in the recombinant microorganism.

The 6MSAS may be derived from Penicillium patulum (or Penicillium griseofulvum), Aspergillus terreus, Aspergillus aculeatus, Aspergillus niger, Aspergillus westerdijkiae, Byssochlamys nivea, Glarea lozoyensis, Penicillium expansum, or Streptomyces antibioticus, but is not limited thereto.

The Sfp may be derived from Bacillus subtilis, Corynebacterium ammoniagenes, Escherichia coli, Homo sapiens, Mycobacterium tuberculosis, Pseudomonas aeruginosa, Ricinus communis, Saccharomyces cerevisiae, Spinacia oleracea, Stigmatella aurantiaca, Streptomyces coelicolor, Streptomyces pneumonia, Streptomyces verticillus, or Vibrio harveyi, but is not limited thereto.

The gene whose expression is decreased compared to that in a wild-type microorganism may be one or more selected from the group consisting of pabA, fabF, xapR and ytcY, but are not limited thereto.

The recombinant microorganism for producing 6-methylsalicylic acid may be characterized in that genes that encode one or more enzymes selected from the group consisting of glucose-6-phosphate dehydrogenase (Zwf), malate dehydrogenase (Mdh), phosphoglycerate dehydrogenase (SerA), acetyl-CoA carboxylase (AccBC and AccD1), glyceraldehyde 3-phosphate dehydrogenase (GapA), phosphoglycerate kinase (Pgk), acetyl-CoA synthetase (Acs), and pyruvate dehydrogenase (PDH: AceEF and Lpd) are additionally introduced or expression of the genes is increased in the recombinant microorganism.

The Zwf, Mdh, SerA, GapA, Pgk, Acs, AceE, AceF and Lpd may be derived from Escherichia coli (E. coli), but are not limited thereto. AccBC and AccD1 may be derived from Corynebacterium glutamicum, but are not limited thereto and can be extended to enzymes corresponding to other microorganism-derived acetyl-CoA carboxylases.

The amino acid sequence of each of enzymes, which are screened and used in the present invention, is as follows, but may have a substitution, deletion or addition of one or more amino acids in the amino acid sequences, and in this case, the resulting mutant amino acid sequence may exhibit a function equal to or better than that in the present invention. For example, it will be obvious that if the structural configuration of the mutant amino acid sequence is identical or similar to the wild-type amino acid sequence, and thus exhibits the same or higher effect on a substrate, the mutant amino acid sequence also falls within the scope of the present invention. In the same context, it will be obvious that besides enzymes used in the present invention, other microorganism-derived enzymes having an identical or similar function can be properly applied to the present invention, and the application of a corresponding enzyme within a range that can be easily applied by a person of ordinary skill in the art falls within the scope of the present invention.

Zwf[SEQ ID NO: 171]:
MAVTQTAQACDLVIFGAKGDLARRKLLPSLYQLEKAGQLNPDTRIIGVGRADWDKA
AYTKVVREALETFMKETIDEGLWDTLSARLDFCNLDVNDTAAFSRLGAMLDQKNRITINYF
AMP
PSTFGAICKGLGEAKLNAKPARVVMEKPLGTSLATSQEINDQVGEYFEECQVYRID
HYLGKETVLNLLALRFANSLFVNNWDNRTIDHVEITVAEEVGIEGRWGYFDKAGQMRDMIQ
NHLLQILCMIAMSPPSDLSADSIRDEKVKVLKSLRRIDRSNVREKTVRGQYTAGFAQGKKV
PGYLEEEGANKSSNTETFVAIRVDIDNWRWAGVPFYLRTGKRLPTKCSEVVVYFKTPELNL
FKESWQDLPQNKLTIRLQPDEGVDIQVLNKVPGLDHKHNLQITKLDLSYSETFNQTHLADA
YERLLLETMRGIQALFVRRDEVEEAWKWVDSITEAWAMDNDAPKPYQAGTWGPVASVAMIT
RDGRSWNEFE
Mdh[SEQ ID NO: 172]:
MKVAVLGAAGGIGQALALLLKTQLPSGSELSLYDIAPVTPGVAVDLSHIPTAVKIK
GFSGEDATPALEGADVVLISAGVARKPGMDRSDLFNVNAGIVKNLVQQVAKTCPKACIGII
TNPVNTTVAIAAEVLKKAGVYDKNKLFGVTTLDIIRSNTFVAELKGKQPGEVEVPVIGGHS
GVTILPLLSQVPGVSFTEQEVADLTKRIQNAGTEVVEAKAGGGSATLSMGQAAARFGLSLV
RALQGEQGVVECAYVEGDGQYARFFSQPLLLGKNGVEERKSIGTLSAFEQNALEGMLDTLK
KDIALGEEFVNK
SerA[SEQ ID NO: 173]:
MAKVSLEKDKIKFLLVEGVHQKALESLRAAGYTNIEFHKGALDDEQLKESIRDAHF
IGLRSRTHLTEDVINAAEKLVAIGCFCIGTNQVDLDAAAKRGIPVFNAPFSNTRSVAELVI
GELLLLLRGVPEANAKAHRGVWNKLAAGSFEARGKKLGIIGYGHIGTQLGILAESLGMYVY
FYDIENKLPLGNATQVQHLSDLLNMSDVVSLHVPENPSTKNMMGAKEISLMKPGSLLINAS
RGTVVDIPALCDALASKHLAGAAIDVFPTEPATNSDPFTSPLCEFDNVLLTPHIGGSTQEA
QENIGLEVAGKLIKYSDNGSTLSAVNFPEVSLPLHGGRRLMHIHENRPGVLTALNKIFAEQ
GVNIAAQYLQTSAQMGYVVIDIEADEDVAEKALQAMKAIPGTIRARLLY
GapA[SEQ ID NO: 174]:
MTIKVGINGFGRIGRIVFRAAQKRSDIEIVAINDLLDADYMAYMLKYDSTHGRFDG
TVEVKDGHLIVNGKKIRVTAERDPANLKWDEVGVDVVAEATGLFLTDETARKHITAGAKKV
VMTGPSKDNTPMFVKGANFDKYAGQDIVSNASCTTNCLAPLAKVINDNFGIIEGLMTTVHA
TTATQKTVDGPSHKDWRGGRGASQNIIPSSTGAAKAVGKVLPELNGKLTGMAFRVPTPNVS
VVDLTVRLEKAATYEQIKAAVKAAAEGEMKGVLGYTEDDVVSTDFNGEVCTSVFDAKAGIA
LNDNFVKLVSWYDNETGYSNKVLDLIAHISK
Pgk[SEQ ID NO: 175]:
MSVIKMTDLDLAGKRVFIRADLNVPVKDGKVTSDARIRASLPTIELALKQGAKVMV
TSHLGRPTEGEYNEEFSLLPVVNYLKDKLSNPVRLVKDYLDGVDVAEGELVVLENVRFNKG
EKKDDETLSKKYAALCDVFVMDAFGTAHRAQASTHGIGKFADVACAGPLLAAELDALGKAL
KEPARPMVAIVGGSKVSTKLTVLDSLSKIADQLIVGGGIANTFIAAQGHDVGKSLYEADLV
DEAKRLLTTCNIPVPSDVRVATEFSETAPATLKSVNDVKADEQILDIGDASAQELAEILKN
AKTILWNGPVGVFEFPNFRKGTEIVANAIADSEAFSIAGGGDTLAAIDLFGIADKISYIST
GGGAFLEFVEGKVLPAVAMLEERAKK
Acs[SEQ ID NO: 176]:
MSQIHKHTIPANIADRCLINPQQYEAMYQQSINVPDTFWGEQGKILDWIKPYQKVK
NTSFAPGNVSIKWYEDGTLNLAANCLDRHLQENGDRTAIIWEGDDASQSKHISYKELHRDV
CRFANTLLELGIKKGDVVAIYMPMVPEAAVAMLACARIGAVHSVIFGGFSPEAVAGRIIDS
NSRLVITSDEGVRAGRSIPLKKNVDDALKNPNVTSVEHVVVLKRTGGKIDWQEGRDLWWHD
LVEQASDQHQAEEMNAEDPLFILYTSGSTGKPKGVLHTTGGYLVYAALTFKYVFDYHPGDI
YWCTADVGWVTGHSYLLYGPLACGATTLMFEGVPNWPTPARMAQVVDKHQVNILYTAPTAI
RALMAEGDKAIEGTDRSSLRILGSVGEPINPEAWEWYWKKIGNEKCPVVDTWWQTETGGFM
ITPLPGATELKAGSATRPFFGVQPALVDNEGNPLEGATEGSLVITDSWPGQARTLFGDHER
FEQTYFSTFKNMYFSGDGARRDEDGYYWITGRVDDVLNVSGHRLGTAEIESALVAHPKIAE
AAVVGIPHNIKGQAIYAYVTLNHGEEPSPELYAEVRNWVRKEIGPLATPDVLHWTDSLPKT
RSGKIMRRILRKIAAGDTSNLGDTSTLADPGVVEKLLEEKQAIAMPS
AceE[SEQ ID NO: 177]:
MSERFPNDVDPIETRDWLQAIESVIREEGVERAQYLIDQLLAEARKGGVNVAAGTG
ISNYINTIPVEEQPEYPGNLELERRIRSAIRWNAIMTVLRASKKDLELGGHMASFQSSATI
YDVCFNHFFRARNEQDGGDLVYFQGHISPGVYARAFLEGRLTQEQLDNFRQEVHGNGLSSY
PHPKLMPEFWQFPTVSMGLGPIGAIYQAKFLKYLEHRGLKDTSKQTVYAFLGDGEMDEPES
KGAITIATREKLDNLVFVINCNLQRLDGPVTGNGKIINELEGIFEGAGWNVIKVMWGSRWD
ELLRKDTSGKLIQLMNETVDGDYQTFKSKDGAYVREHFFGKYPETAALVADWTDEQIWALN
RGGHDPKKIYAAFKKAQETKGKATVILAHTIKGYGMGDAAEGKNIAHQVKKMNMDGVRHIR
DRFNVPVSDADIEKLPYITFPEGSEEHTYLHAQRQKLHGYLPSRQPNFTEKLELPSLQDFG
ALLEEQSKEISTTIAFVRALNVMLKNKSIKDRLVPIIADEARTFGMEGLFRQIGIYSPNGQ
QYTPQDREQVAYYKEDEKGQILQEGINELGAGCSWLAAATSYSTNNLPMIPFYIYYSMFGF
QRIGDLCWAAGDQQARGFLIGGTSGRTTLNGEGLQHEDGHSHIQSLTIPNCISYDPAYAYE
VAVIMHDGLERMYGEKQENVYYYITTLNENYHMPAMPEGAEEGIRKGIYKLETIEGSKGKV
QLLGSGSILRHVREAAEILAKDYGVGSDVYSVTSFTELARDGQDCERWNMLHPLETPRVPY
IAQVMNDAPAVASTDYMKLFAEQVRTYVPADDYRVLGTDGFGRSDSRENLRHHFEVDASYV
VVAALGELAKRGEIDKKVVADAIAKFNIDADKVNPRLA
AceF[SEQ ID NO: 178]:
MAIEIKVPDIGADEVEITEILVKVGDKVEAEQSLITVEGDKASMEVPSPQAGIVKE
IKVSVGDKTQTGALIMIFDSADGAADAAPAQAEEKKEAAPAAAPAAAAAKDVNVPDIGSDE
VEVTEILVKVGDKVEAEQSLITVEGDKASMEVPAPFAGTVKEIKVNVGDKVSTGSLIMVFE
VAGEAGAAAPAAKQEAAPAAAPAPAAGVKEVNVPDIGGDEVEVTEVMVKVGDKVAAEQSLI
TVEGDKASMEVPAPFAGVVKELKVNVGDKVKTGSLIMIFEVEGAAPAAAPAKQEAAAPAPA
AKAEAPAAAPAAKAEGKSEFAENDAYVHATPLIRRLAREFGVNLAKVKGTGRKGRILREDV
QAYVKEAIKRAEAAPAATGGGIPGMLPWPKVDFSKFGEIEEVELGRIQKISGANLSRNWVM
IPHVTHFDKTDITELEAFRKQQNEEAAKRKLDVKITPVVFIMKAVAAALEQMPRFNSSLSE
DGQRLTLKKYINIGVAVDTPNGLVVPVFKDVNKKGIIELSRELMTISKKARDGKLTAGEMQ
GGCFTISSIGGLGTTHFAPIVNAPEVAILGVSKSAMEPVWNGKEFVPRLMLPISLSFDHRV
IDGADGARFITIINNTLSDIRRLVM
Lpd[SEQ ID NO: 179]:
MSTEIKTQVVVLGAGPAGYSAAFRCADLGLETVIVERYNTLGGVCLNVGCIPSKAL
LHVAKVIEEAKALAEHGIVFGEPKTDIDKIRTWKEKVINQLTGGLAGMAKGRKVKVVNGLG
KFTGANTLEVEGENGKTVINFDNAIIAAGSRPIQLPFIPHEDPRIWDSTDALELKEVPERL
LVMGGGIIGLEMGTVYHALGSQIDVVEMFDQVIPAADKDIVKVFTKRISKKFNLMLETKVT
AVEAKEDGIYVTMEGKKAPAEPQRYDAVLVAIGRVPNGKNLDAGKAGVEVDDRGFIRVDKQ
LRTNVPHIFAIGDIVGQPMLAHKGVHEGHVAAEVIAGKKHYFDPKVIPSIAYTEPEVAWVG
LTEKEAKEKGISYETATFPWAASGRAIASDCADGMTKLIFDKESHRVIGGAIVGTNGGELL
GEIGLAIEMGCDAEDIALTIHAHPTLHESVGLAAEVFEGSITDLPNPKAKKK
AccBC[SEQ ID NO: 180]:
VSVETRKITKVLVANRGEIAIRVFRAARDEGIGSVAVYAEPDADAPFVSYADEAFA
LGGQTSAESYLVIDKIIDAARKSGADAIHPGYGFLAENADFAEAVINEGLIWIGPSPESIR
SLGDKVTARHIADTAKAPMAPGTKEPVKDAAEVVAFAEEFGLPIAIKAAFGGGGRGMKVAY
KMEEVADLFESATREATAAFGRGECFVERYLDKARHVEAQVIADKHGNVVVAGTRDCSLQR
RFQKLVEEAPAPFLTDDQRERLHSSAKAICKEAGYYGAGTVEYLVGSDGLISFLEVNTRLQ
VEHPVTEETTGIDLVREMFRIAEGHELSIKEDPAPRGHAFEFRINGEDAGSNFMPAPGKIT
SYREPQGPGVRMDSGVVEGSEISGQFDSMLAKLIVWGDTREQALQRSRRALAEYVVEGMPT
VIPFHQHIVENPAFVGNDEGFEIYTKWIEEVWDNPIAPYVDASELDEDEDKTPAQKVVVEI
NGRRVEVALPGDLALGGTAGPKKKAKKRRAGGAKAGVSGDAVAAPMQGTVIKVNVEEGAEV
NEGDTVVVLEAMKMENPVKAHKSGTVTGLTVAAGEGVNKGVVLLEIK
AccD1[SEQ ID NO: 181]:
MTISSPLIDVANLPDINTTAGKIADLKARRAEAHFPMGEKAVEKVHAAGRLTARER
LDYLLDEGSFIETDQLARHRTTAFGLGAKRPATDGIVTGWGTIDGREVCIFSQDGTVFGGA
LGEVYGEKMIKIMELAIDTGRPLIGLYEGAGARIQDGAVSLDFISQTFYQNIQASGVIPQI
SVIMGACAGGNAYGPALTDFVVMVDKTSKMFVTGPDVIKTVTGEEITQEELGGATTHMVTA
GNSHYTAATDEEALDWVQDLVSFLPSNNRSYTPLEDFDEEEGGVEENITADDLKLDEIIPD
SATVPYDVRDVIECLTDDGEYLEIQADRAENVVIAFGRIEGQSVGFVANQPTQFAGCLDID
SSEKAARFVRTCDAFNIPIVMLVDVPGFLPGAGQEYGGILRRGAKLLYAYGEATVPKITVT
MRKAYGGAYCVMGSKGLGSDINLAWPTAQIAVMGAAGAVGFIYRKELMAADAKGLDTVALA
KSFEREYEDHMLNPYHAAERGLIDAVILPSETRGQISRNLRLLKHKNVTRPARKHGNMPL

The present invention is also directed to a method of producing 6-methylsalicylic acid, the method comprising the steps of:

(a) culturing the above-described recombinant microorganism; and

(b) recovering the 6-methylsalicylic acid from the cultured microorganism.

The method of producing 6-methylsalicylic acid of the present invention may comprises culturing the recombinant microorganism by adding 1-50 g/L of glucose or 1-100 g/L of glycerol as a carbon source.

The present invention is also directed to a recombinant microorganism for producing aloesone in which a gene that encodes aloesone synthase is additionally introduced or expression of the gene is increased in the recombinant microorganism. In the present invention, the aloesone synthase may be ALS or PKS3, the ALS may be derived from Rheum palmatum, and the PKS3 may be derived from Aloe arborescens, but aloesone synthase other than the aloesone synthase can be applied to the present invention.

The gene whose expression is decreased compared to that in a wild-type microorganism may be pabA, but are not limited thereto.

The recombinant microorganism for producing aloesone may be characterized in that genes that encode one or more enzymes selected from the group consisting of glucose-6-phosphate dehydrogenase (Zwf), malate dehydrogenase (Mdh), phosphoglycerate dehydrogenase (SerA), acetyl-CoA carboxylase (AccBC and AccD1), glyceraldehyde 3-phosphate dehydrogenase (GapA), phosphoglycerate kinase (Pgk), acetyl-CoA synthetase (Acs), and pyruvate dehydrogenase (PDH: AceEF and Lpd) are additionally introduced or expression of the genes is increased in the recombinant microorganism.

The Zwf, Mdh, SerA, GapA, Pgk, Acs, AceE, AceF and Lpd may be derived from Escherichia coli (E. coli), but are not limited thereto. AccBC and AccD1 may be derived from Corynebacterium glutamicum, but are not limited thereto and can be extended to enzymes corresponding to other microorganism-derived acetyl-CoA carboxylases.

The present invention is also directed to a method of producing aloesone, the method comprising the steps of:

(a) culturing the above-described recombinant microorganism; and

(b) recovering the aloesone from the cultured microorganism.

The method of producing aloesone of the present invention may comprises culturing the recombinant microorganism by adding 1-50 g/L of glucose or 1-100 g/L of glycerol as a carbon source.

The present invention is also directed to a recombinant microorganism for producing resveratrol in which genes that encode tyrosine ammonia-lyase (TAL), 4-coumarate:CoA ligase (4CL) and stilbene synthase (STS) are additionally introduced or expression of the genes is increased in the recombinant microorganism.

TAL may be derived from Rhodobacter capsulatus, Clitoria ternatea, Fragaria x ananassa, Rhodobacter sphaeroides, Zea mays or Saccharothrix espanaensis,

4CL may be derived from Arabidopsis thaliana, Streptomyces coelicolor, Acetobacterium woodii, Agastache rugose, Avena sativa, Camellia sinensis, Centaurium erythraea, Cephalocereus senilis, Cocos nucifera, Eriobotrya japonica, Erythrina cristagalli, Forsythia sp., Fragaria x ananassa, Glycine max, Gossypium hirsutum, Hibiscus cannabinus, Larix cajanderi, Larix gmelinii, Larix kaempferi, Larix kamtschatica, Larix sibirica, Larix sukaczewii, Lithospermum erythrorhizon, Lolium perenne, Lonicera japonica, Metasequoia glyptostroboides, Nicotiana tabacum, Ocimum basilicum, Ocimum tenuiflorum, Oryza sativa, Paulownia tomentosa, Petroselinum crispum, Phyllostachys bambusoides, Physcomitrella patens, Picea abies, Pinus radiate, Pinus taeda, Pisum sativum, Platycladus orientalis, Polyporus hispidus, Populus tomentosa, Populus tremuloides, Populus x canadensis, Prunus avium, Pueraria montana, Robinia pseudoacacia, Ruta graveolens, Saccharomyces cerevisiae, Salix babylonica, Solanum lycopersicum, Solanum tuberosum, Sorbus aucuparia, Triticum aestivum or Vitis vinifera, and

STS may be derived from Arachis hypogaea, Pinus densiflora, Pinus massoniana, Pinus strobes, Polygonum cuspidatum, Psilotum nudum or Vitis vinifera, but are not limited thereto.

The 4-coumarate:CoA ligase may be either a mutant enzyme in which amino acids are mutated to I250L/N404K/I461V in the amino acid sequence represented by SEQ ID NO: 128 or a mutant enzyme in which amino acids are mutated to A294G/A318G in the amino acid sequence represented by SEQ ID NO: 131, but is not limited thereto.

The gene whose expression is decreased compared to that in a wild-type microorganism may be one or more selected from the group consisting of pabA, yfiD, mqo, xapR, purB, fabH, fabF, ytcY, argC, nudD, araA, fadR, cytR, fmt and pyrF, but are not limited thereto.

The present invention is also directed to a method of producing resveratrol, the method comprising the steps of:

(a) culturing the above-described recombinant microorganism; and

(b) recovering the resveratrol from the cultured microorganism.

The method of producing resveratrol of the present invention may comprises culturing the recombinant microorganism by adding 1-50 g/L of glucose or 1-100 g/L of glycerol as a carbon source.

The present invention is also directed to a recombinant microorganism for producing naringenin in which genes that encode tyrosine ammonia-lyase (TAL), 4-coumarate:CoA ligase (4CL), chalcone synthase (CHS), and chalcone isomerase (CHI) are additionally introduced in the above-described recombinant microorganism or expression of the genes is increased.

TAL may be derived from Rhodobacter capsulatus, Clitoria ternatea, Fragaria x ananassa, Rhodobacter sphaeroides, Zea mays or Saccharothrix espanaensis,

4CL may be derived from Arabidopsis thaliana, Streptomyces coelicolor, Acetobacterium woodii, Agastache rugose, Avena sativa, Camellia sinensis, Centaurium erythraea, Cephalocereus senilis, Cocos nucifera, Eriobotrya japonica, Erythrina cristagalli, Forsythia sp., Fragaria x ananassa, Glycine max, Gossypium hirsutum, Hibiscus cannabinus, Larix cajanderi, Larix gmelinii, Larix kaempferi, Larix kamtschatica, Larix sibirica, Larix sukaczewii, Lithospermum erythrorhizon, Lolium perenne, Lonicera japonica, Metasequoia glyptostroboides, Nicotiana tabacum, Ocimum basilicum, Ocimum tenuiflorum, Oryza sativa, Paulownia tomentosa, Petroselinum crispum, Phyllostachys bambusoides, Physcomitrella patens, Picea abies, Pinus radiate, Pinus taeda, Pisum sativum, Platycladus orientalis, Polyporus hispidus, Populus tomentosa, Populus tremuloides, Populus x canadensis, Prunus avium, Pueraria montana, Robinia pseudoacacia, Ruta graveolens, Saccharomyces cerevisiae, Salix babylonica, Solanum lycopersicum, Solanum tuberosum, Sorbus aucuparia, Triticum aestivum or Vitis vinifera,

CHS may be derived from Freesia hybrid cultivar, Medicago sativa, Physcomitrella patens, Plagiochasma appendiculatum, Triticum aestivum, Vitis vinifera, Citrus sinensis, Arabidopsis thaliana, Avena sativa, Daucus carota, Fagopyrum esculentum, Glycine max, Glycyrrhiza echinata, Humulus lupulus, Hypericum androsaemum, Petroselinum crispum, Physcomitrella patens, Rubus idaeus, Scutellaria baicalensis, Xanthisma gracile, Cosmos sulphureus, Gerbera hybrid cultivar, Hordeum vulgare, Juglans sp., Phaseolus vulgaris, Pueraria montana, Secale cereal, Silene sp., Sinapis alba, Spinacia oleracea, Stellaria longipes, Tulipa hybrid cultivar, Verbena sp., or Petunia x hybrida, and

CHI may be derived from Perilla frutescens, Ginkgo biloba, Trigonella foenumgraecum, Medicago sativa, Scutellaria baicalensis, Glycine max, Cephalocereus senilis, Citrus sinensis, Glycyrrhiza echinata, Glycyrrhiza uralensis, Lilium candidum, Morella rubra, Petunia x hybrid, Phaseolus vulgaris, Soja hispida, Tulipa hybrid cultivar, Arabidopsis thaliana or Nicotiana tabacum, but are not limited thereto.

4-coumarate:CoA ligase may be a mutant enzyme in which amino acids are mutated to I250L/N404K/I461V in the amino acid sequence represented by SEQ ID NO: 128, but is not limited thereto.

The gene whose expression is decreased compared to that in a wild-type microorganism may be one or more selected from the group consisting of fadR, hycI and xapR.

The present invention is also directed to a method of producing naringenin, the method comprising the steps of:

(a) culturing the above-described recombinant microorganism; and

(b) recovering the naringenin from the cultured microorganism.

The method of producing naringenin of the present invention may comprises culturing the recombinant microorganism by adding 1-50 g/L of glucose or 1-100 g/L of glycerol as a carbon source.

In the present invention, “regulation” of a gene includes all gene deletion, expression inhibition, expression promotion, knockdown, promoter replacement, and inducing expression of the gene by a technique such as a regulatory mechanism, and is meant to include evolving or mutating one or more of enzymes present in the biosynthetic pathway.

In the present invention, “knockdown” means significantly reducing the expression level of a gene so as to reduce the function of the gene, unlike “knockout” that completely blocks gene expression. It can be regulated at the gene transcript level or at the protein level. However, since the present invention is significant in inhibiting or reducing the expression of a gene encoding an enzyme involved in the corresponding pathway, the use of any of “knockdown” and “knockout” can achieve the desired object.

As used herein, the term “vector” means a DNA construct containing a DNA sequence operably linked to a suitable control sequence capable of effecting the expression of the DNA in a suitable host. The vector may be a plasmid, a phage particle, or simply a potential genomic insert. Once incorporated into a suitable host, the vector may replicate and function independently of the host genome, or may in some instances, integrate into the genome itself. In the present specification, “plasmid” and “vector” are sometimes used interchangeably, as the plasmid is the most commonly used form of vector. For the purpose of the present invention, the plasmid vector is preferably used. A typical plasmid vector which can be used for this purpose contains: (a) a replication origin by which replication occurs efficiently such that several hundred plasmid vectors per host cell are created; (b) an antibiotic-resistant gene by which host cells transformed with the plasmid vector can be selected; and (c) restriction enzyme cutting sites into which foreign DNA fragments can be inserted. Even if suitable restriction enzyme cutting sites are not present in the vector, the use of a conventional synthetic oligonucleotide adaptor or linker enables the easy ligation between the vector and the foreign DNA fragments. After ligation, the vector should be transformed into suitable host cells. The transformation can be easily achieved by the calcium chloride method or electroporation (Neumann, et al., EMBO J., 1:841, 1982).

The promoter of the vector may be constructive or inductive, and may be further modified to achieve the effects of the present invention. Furthermore, the expression vector includes a selective marker for selecting a host cell containing the vector, and a replicable expression vector includes a replication origin. The vector may be self-replicating, or may be integrated into the host genome DNA. Preferably, a gene inserted into the vector is irreversibly fused into the genome of the host cell, such that the expression of the gene in the cells is stably continued for a long period of time).

A nucleotide sequence is “operably linked” when it is placed into a functional relationship with another nucleotide sequence. The nucleotide sequence may be a gene and a control sequence(s) linked to be capable of expressing the gene when it binds to a control sequence(s) (e.g., transcription-activating protein). For example, DNA for a pre-sequence or a secretory leader is operably linked to DNA encoding polypeptide when expressed as pre-protein participating in secretion of polypeptide; a promoter or an enhancer is operably linked to a coding sequence when affecting the transcription of the sequence; and a ribosome binding site (RBS) is operably linked to a coding sequence when affecting the transcription of the sequence, or to a coding sequence when arranged to facilitate translation. Generally, the term “operably linked” means that the DNA linked sequences are contiguous, and in the case of the secretory leader, are contiguous and present in a reading frame. However, an enhancer is not necessarily contiguous. The linkage between these sequences is performed by ligation at a convenient restriction enzyme site. However, when the site does not exist, a synthetic oligonucleotide adaptor or a linker is used according to a conventional method.

As is well known in the art, in order to increase the expression level of a transfected gene in a host cell, a corresponding gene should be operably linked to transcription and translation expression control sequences which are operated in a selected expression host. Preferably, the expression control sequences and the corresponding gene are included in one recombinant vector together with a bacterial selection marker and a replication origin. When a host cell is a eukaryotic cell, a recombinant vector should further include an expression marker which is useful in a eukaryotic expression host.

The host cell transformed by the aforementioned recombinant vector constitutes another aspect of the present invention. As used herein, the term “transformation” means that DNA can be replicated as a factor outside of chromosome or by means of completion of the entire chromosome by introducing DNA as a host.

Of course, it should be understood that all vectors and expression control sequences do not equally function to express DNA sequences according to the present invention. Similarly, all hosts do not equally function with respect to the same expression system. However, one skilled in the art may appropriately select from among various vectors, expression control sequences, and hosts without either departing from the scope of the present invention or bearing excessive experimental burden. For example, a vector must be selected considering a host, because the vector must be replicated in the host. Specifically, the copy number of the vector, the ability of regulating the copy number and the expression of other protein encoded by the corresponding vector (e.g., the expression of an antibiotic marker) should also be considered.

In addition, the gene introduced in the present invention may be introduced into the genome of a host cell and present as a chromosomal factor. It will be obvious to those skilled in the art to which the present invention pertains that even when the gene is inserted into the genomic chromosome of a host cell, it has the same effect as when the recombinant vector is introduced into the host cell as described above.

### EXAMPLES

Hereinafter, the present invention will be described in further detail with reference to examples. It will be obvious to a person having ordinary skill in the art that these examples are for illustrative purposes only and are not to be construed to limit the scope of the present invention.

### Example 1. Construction of Type III Polyketide Synthase-Based Malonyl-CoA Biosensor

1.1. Performance Test for Various Type III Polyketide Synthases

The restriction enzymes used in this Example and the following Examples were purchased from New England Labs (USA) or Enzynomics (Korea), and the PCR polymerase was purchased from Biofact (Korea). Others are marked separately. In addition, the introduced exogenous genes in this Example and the following Examples are summarized in Table 1 below together with the accession numbers in a database containing information thereon and the sequences thereof. In addition, unless otherwise specified, the E. coli strain used in cloning was a DH5a strain which was cultured in a LB medium (per liter: 10 g tryptone, 5 g yeast extract and 10 g NaCl) or on an LB agar plate (1/5%, v/v). Furthermore, if necessary, a suitable concentration of an antibiotic (50 μg/mL kanamycin, 100 μg/mL ampicillin and/or 100 μg/mL spectinomycin) was added.

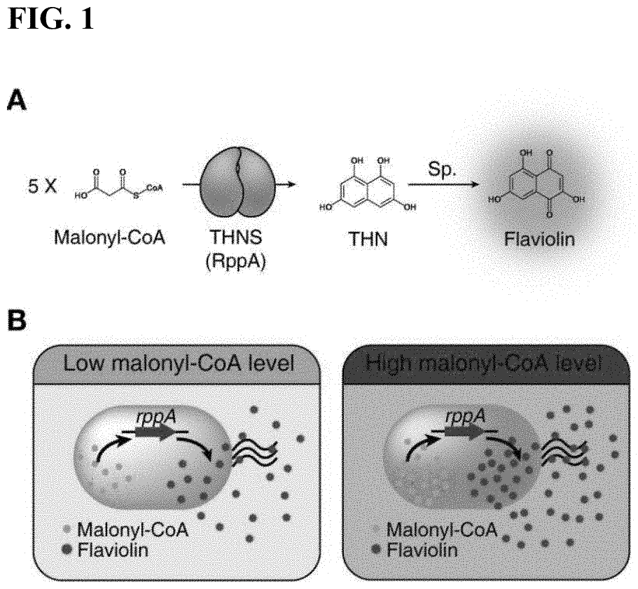

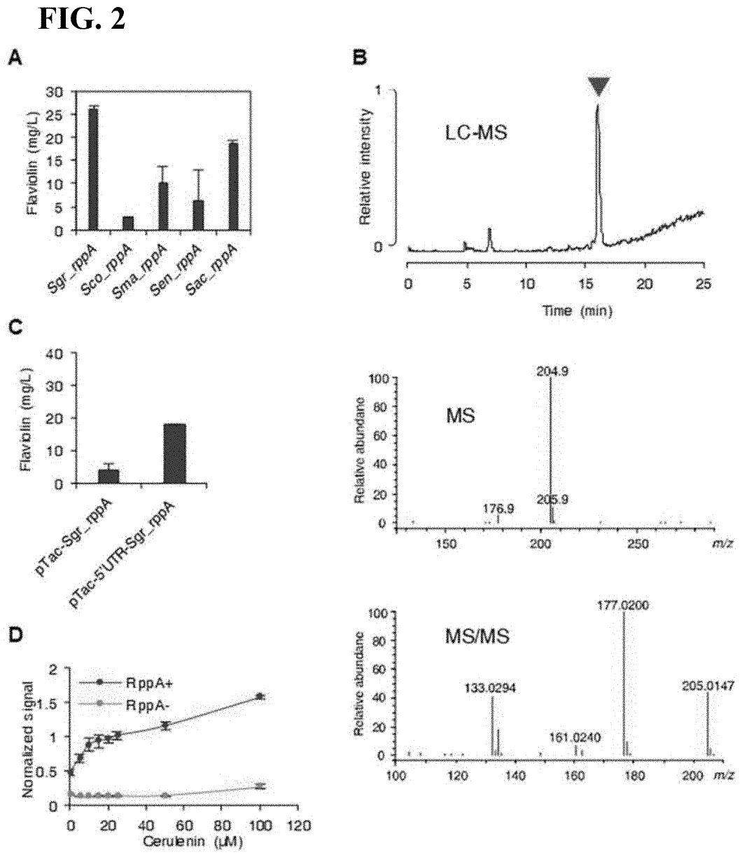

In the present invention, red-colored flaviolin was produced from five molecules of malonyl-CoA using type III polyketide synthase RppA, and used as an indicator of intracellular malonyl-CoA level (FIG. 1). To this end, the performances of various III polyketide synthases were compared and tested. Type III polyketide synthase-encoding rppA genes from a total of five Actinomycetes strains (Streptomyces griseus, Streptomyces coelicolor, Streptomyces avermitilis, Saccharopolyspora erythraea, Streptomyces aculeolatus) were cloned, and each of the genes was transformed into an E. coli BL21(DE3) strain. Then, each of the constructed strains was inoculated into a flask containing M9 minimal medium supplemented with 10 g/L of glucose, and then treated with 0.5 mM isopropyl-β-D-1-thiogalactopyranoside (IPTG) in an initial exponential phase. Next, each strain was cultured for 48 hours, and the production of flaviolin was observed. At this time, the highest flaviolin production was observed in the strain in which the S. griseus-based enzyme Sgr_RppA was expressed (25.7 mg/L; FIG. 2A). The constructed plasmids were based on pET-30a(+) (Novagen) and named pET-Sgr_rppA, pET-Sco_rppA, pET-Sma_rppA, pET-Sen_rppA, and pET-Sac_rppA, respectively. For construction of pET-Sgr_rppA, the rppA gene was amplified using the genomic DNA of S. griseus as a template and the primer pair of SEQ ID NO: 1 and SEQ ID NO: 2. The gene was cloned into the pET-30a(+) plasmid at EcoRI and BamHI sites using Gibson assembly (Gibson D G, et al. (2009), Nature Methods 6:343-345). Other plasmids were also constructed by the same method as described above. The M9 minimal medium contained, per liter, the following components: 12.8 g Na₂HPO₄.7H₂O, 3 g KH₂PO₄, 0.5 g NaCl, 1 g NH₄Cl, 2 mM MgSO₄, 0.1 mM CaCl₂).

[SEQ ID NO: 1]
5′-CTTTAAGAAGGAGATATACATATGGCGACCCTGTGCCGACC-3′
[SEQ ID NO: 2]
5′-CTTGTCGACGGAGCTCGAATTCATTAGCCGGACAGCGCAACGC-3′

TABLE 1
Information on exogenous genes proposed in the present invention

| | | Accession |
|---|---|---|
| Gene | Organism | Number * |
| Sgr_rppA | Streptomyces griseus | BAE07216 |
| Sco_rppA | Streptomyces coelicolor | CAC01488 |
| Sma_rppA | Streptomyces avermitilis | BAC74842 |
| Sen_rppA | Saccharopolyspora erythraea | AAL78053 |
| Sac_rppA | Streptomyces aculeolatus | ABS50451 |
| whiE_ORFII | Streptomyces coelicolor | CAB45605 |
| AaPCS | Aloe arborescens | AAX35541 |
| AaOKS | Aloe arborescens | AAT48709 |
| AaPKS4 | Aloe arborescens | ACR19997 |
| AaPKS5 | Aloe arborescens | ACR19998 |
| sfp | Bacillus subtilis | ABV89950 |
| Pg6MSAS | Penicillium griseofulvum | CAA39295 |
| RpALS | Rheum palmatum | AAS87170 |
| AaPKS3 | Aloe arborescens | ABS72373 |
| SeTAL | Saccharothrix espanaensis | CCH33126 |
| At4CL1 | Arabidopsis thaliana | AAQ86588 |
| At4CL3 | Arabidopsis thaliana | AAQ86589 |
| At4CL4 | Arabidopsis thaliana | Q9LU36 |
| Sc4CL | Streptomyces coelicolor | CAB95894 |
| PhCHS | Petunia × hybrida | AGZ04577 |
| AtCHI | Arabidopsis thaliana | NP_191072 |
| VvSTS^{†} | Vitis vinifera | CAA54221 |

* depicts NCBI numbers for enzymes
^{†}codon-optimized for E. coli K-12 MG1655.

In FIG. 1A, malonyl-CoA is converted to THN by the RppA enzyme. Then, THN is converted to flaviolin by a spontaneous oxidation reaction. WhiE_ORFIII was reported to involved in this final step (Austin M B, et al. (2004), J Biol Chem 279:45162-45174.), and hence, in the present invention, the corresponding gene was additionally cloned into pET-30a(+), but it caused reduced flaviolin production. This demonstrates that the rppA single gene is sufficient for flaviolin production. The produced flaviolin was confirmed by LC-MS and MS/MS (FIG. 2B). Thereafter, the rppA expression platform was established in a tac promoter-based plasmid for its use in all E. coli strains beyond the conventional T7 promoter-based system. To this end, a pTacCDFS plasmid based on a pCDFDuet-1 (Novagen) plasmid was first constructed, which contains a CDF replication origin and has spectinomycin antibiotic resistance and a tac promoter-based gene expression cassette. To this end, a DNA fragment containing the tac promoter was amplified by PCR using a pTac15K plasmid (Lee S Y, et al. (2008), US patent 20110269183) as a template and the primers of SEQ ID NO: 3 and SEQ ID NO: 4, and the corresponding plasmid was linearized using a pCDFDuet-1 plasmid as a template and the primers of SEQ ID NO: 5 and SEQ ID NO: 6. The produced two DNA fragments were assembled using Gibson assembly, thereby constructing a pTacCDFS plasmid. A pTrcCDFS plasmid was also constructed for further use, and this plasmid is the same as the pTacCDFS plasmid, except that it contains the trc promoter. In addition, the pTrcCDFS plasmid was constructed in the same manner as the pTacCDFS plasmid, except that the DNA fragment containing the trc promoter was amplified by PCR using the pTrc99A plasmid (Lee S Y, et al. (2008), US patent 20110269183) as a template. Thereafter, the rppA expression plasmid pTac-Sgr_rppA based on the pTacCDFS plasmid was constructed by the following method. First, the rppA gene was amplified from the genomic DNA of S. griseus using the primers of SEQ ID NO: 7 and SEQ ID NO: 8, and the pTacCDFS plasmid was linearized using the primers of SEQ ID NO: 9 and SEQ ID NO: 10. The two produced DNA fragments were assembled using Gibson assembly, thereby constructing a pTac-Sgr_rppA plasmid. Furthermore, for optimized expression of rppA, optimization of the 5′ untranslated region (5′UTR) was performed. The 5′UTR DNA sequence was designed using the previously reported UTR designer (url: https://sbi.postech.ac.kr/utr_designer/). For construction of the corresponding primer pTac-5′UTR-Sgr_rppA, inverse PCR was performed using pTac-Sgr_rppA as a template and the primers of SEQ ID NO: 9 and SEQ ID NO: 11. The produced linearized plasmid was treated with DpnI to remove the template, and ligated by T4 polynucleotide kinase (PNK) (Enzynomics, Korea) and T4 ligase (Elpis Biotech, Korea) treatment. The resulting plasmid was introduced into an E. coli BL21(DE3) strain and cultured in M9 minimal medium, thereby producing 17.8 mg/L of flaviolin (FIG. 2C).

[SEQ ID NO: 3]
5'-CGACTCCTGCATTAGGAAATGACTGCACGGTGCACCAATG-3'
[SEQ ID NO: 4]
5'-GCGTTTCACTTCTGAGTTCG-3'
[SEQ ID NO: 5]
5'-CGAACTCAGAAGTGAAACGCCTGAAACCTCAGGCATTTGAG-3'
[SEQ ID NO: 6]
5'-ATTTCCTAATGCAGGAGTCG-3'
[SEQ ID NO: 7]
5'-CAATTTCACACAGGAAACAGAATGGCGACCCTGTGCCGACC-3'
[SEQ ID NO: 8]
5'-CTCTAGAGGATCCCCGGGTACCATTAGCCGGACAGCGCAACGC-3'
[SEQ ID NO: 9]
5'-GGTACCCGGGGATCCTCTAGAG-3'
[SEQ ID NO: 10]
5'-TCTGTTTCCTGTGTGAAATTG-3'
[SEQ ID NO: 11]
5'-GATGCTCCTTTCTTGTTATTGAAATTGTTATCCGCTCACAATTC-3'

1.2. Examination of Applicability of Type III Polyketide Synthase-Based Malonyl-CoA Biosensor

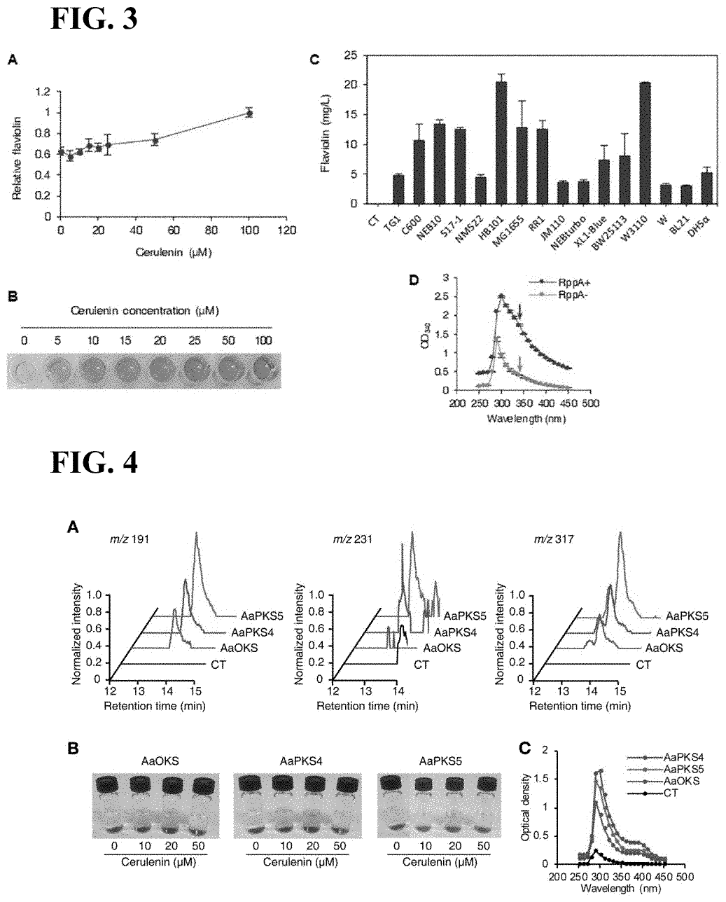

After construction of a malonyl-CoA biosensor based on S. griseus RppA, the following experiment was performed in order to examine the applicability of the biosensor. First, the signal of the RppA malonyl-CoA biosensor was defined as the absorbance of culture supernatant at 340 nm (FIG. 3D). Generally, for characterizing a biosensor, different concentrations of a target compound has to be added to examine whether an increased signal occur. However, because malonyl-CoA is a substance that cannot pass through the cell membrane, adding malonyl-CoA to medium is meaningless. Thus, cerulenin (Sigma-aldrich, USA) known to block fatty acid biosynthetic pathways was used to indirectly characterize the biosensor (Omura S (1976), Bacteriol Rev 40:681-697.). Cerulenin blocks fatty acid biosynthetic pathways, and thus leads to the accumulation of malonyl-CoA, a fatty acid precursor. Thus, the concentration of malonyl-CoA in E. coli according to addition of different concentrations of cerulenin as described in previous studies was measured (Xu P, Li L, Zhang F, Stephanopoulos G, Koffas M (2014), Proc Natl Acad Sci USA 111:11299-11304). An E. coli BL21(DE3) strain containing the plasmid pTac-5′UTR-Sgr_rppA was inoculated into 3 mL of LB medium in a 14-mL disposable Falcon round-bottom tube. Next, the strain was cultured in an incubator at 30° C. and 220 rpm for 16 hours, and then inoculated into 3 mL of modified R/2 medium (pH 6.8) in another 14-mL disposable Falcon round-bottom tube. The modified R/2 medium was prepared to contain, per liter, the following components: 3 g yeast extract, 6.75 g KH₂PO4, 2 g (NH₄)₂HPO₄, 0.8 g MgSO₄.7H₂O, 3 g (NH₄)₂SO₄, 0.85 g citric acid, 5 mL trace metal solution [per 1 L of 0.1 M HCl: 10 g FeSO₄.7H₂O, 2.25 g ZnSO₄.7H₂O, 0.58 g MnSO₄.5H₂O, 1 g CuSO₄.5H₂O, 0.1 g (NH₄)₆Mo₇O₂₄.4H₂O, 0.02 g Na₂B₄O₇.10H₂O, 2 g CaCl₂.2H₂O]. 20 g/L of glucose, 100 μg/mL of spectinomycin, 0.2 mM IPTG, and a suitable concentration of cerulenin were added thereto. The concentrations of cerulenin added were as follows: 0, 5, 10, 15, 20, 25, 50, and 100 μM. The inoculated cells were cultured in an incubator at 30° C. and 220 rpm for 16 hours, and then the growth (OD₆₀₀) of the cells and the absorbance at 340 nm of the culture supernatant were measured. The results of the measurement are shown in FIG. 2D. At this time, it could be seen that the RppA-expressing strain generated a normalized signal, which obviously increased as the concentration of cerulenin increased, compared to the strain that does not express RppA. As shown in FIG. 3A, it could be confirmed that the normalized production of flaviolin also increased with increasing cerulenin concentration, as observed by the naked eyes (FIG. 3B). In addition, it was confirmed that when the pTac-5′UTR-Sgr_rppA plasmid was introduced into 16 different E. coli strains (Table 2), flaviolin was produced from all the strains. This demonstrates that the malonyl-CoA biosensor proposed in the present invention is applicable to all E. coli strains (FIG. 3C).

TABLE 2

| Strain | Descrotion |
|---|---|
| DHS [symbol] | [symbol] |
| TG1 | [symbol] |
| C600 | [symbol] |
| NEB10 | [symbol] |
| S17-1 | Mobilizing donor strain, pro recA, which has |
| | an RP4 derivative intergrated into |
| | the chromosome |
| NM522 | [symbol] |
| HB101 | [symbol] |
| NG1655 | [symbol] |
| RP1 | [symbol] |
| JM110 | [symbol] |
| NEBturbo | [symbol] |
| XL1-Blue | [symbol] |
| [symbol] w25113 | [symbol] |
| W3110 | [symbol] |
| W3110(DE3) | [symbol] |
| W | ATCC 9637 |
| BL21(DE3) | [symbol] |
| BAP1 | BL21 (DE3) [symbol] |
| [symbol] | BL21 (DE3) Δ [symbol] Δ [symbol] |
| [symbol] | [symbol] |

[symbol]

indicates data missing or illegible when filed

Furthermore, in order to demonstrate that other type III polyketide synthases in addition to the S. griseus RppA-based malonyl-CoA biosensor are also applicable as malonyl-CoA biosensors, three type III polyketide synthases were tested. To this end, Aloe arborescens type III polyketide synthase (octaketide synthase, OKS)[SEQ ID NO: 103], Aloe arborescens type III polyketide synthase (octaketide synthase 2, PKS4)[SEQ ID NO: 105], and Aloe arborescens type III polyketide synthase (octaketide synthase 3, PKS5)[SEQ ID NO: 106] were tested, the plasmids pET-AaOKS, pET-AaPKS4 and pET-AaPKS5, each containing a gene encoding each of the enzymes, were constructed as follows. First, AaOKS was amplified by PCR using a synthesized AaOKS gene (Integrated DNA Technologies, Inc., USA) as a template and the primers of SEQ ID NO: 137 and SEQ ID NO: 138, and then inserted into the a pET-30a(+) plasmid at NdeI and EcoRI sites. Other plasmids were also constructed in the same method as described above, except that AaPKS4 was amplified using the primers of SEQ ID NO: 141 and SEQ ID NO: 142 and AaPKS5 was amplified using the primers of SEQ ID NO: 143 and SEQ ID NO: 144.

[SEQ ID NO: 135]
5′-CTTTAAGAAGGAGATATACATATGAGTTCACTCTCCAACTCTC-3′
[SEQ ID NO: 136]
5′-CTTGTCGACGGAGCTCGAATTCATTACATGAGAGGCAGGCTGTG-3′
[SEQ ID NO: 137]
5′-CTTTAAGAAGGAGATATACATATGAGTTCACTCTCCAACGCTTC-3′
[SEQ ID NO: 138]
5′-CTTGTCGACGGAGCTCGAATTCATTACATGAGAGGCAGGCTGTG-3′
[SEQ ID NO: 139]
5′-CTTTAAGAAGGAGATATACATATGGGTTCACTCTCCGACTCTAC-3′
[SEQ ID NO: 140]
5′-CTTGTCGACGGAGCTCGAATTCATTACACAGGAGGCAGGCTATG-3′
[SEQ ID NO: 141]
5′-CTTTAAGAAGGAGATATACATATGGGTTCACTCTCCAACTAC-3′
[SEQ ID NO: 142]
5′-CTTGTCGACGGAGCTCGAATTCATTACACAGGGGGCAGGCTG-3′
[SEQ ID NO: 143]
5′-CTTTAAGAAGGAGATATACATATGGGTTCGATCGCCGAG-3′
[SEQ ID NO: 144]
5′-CTTGTCGACGGAGCTCGAATTCATTAGACAGGAGGCAGGCTG-3′
[SEQ ID NO: 145]
5′-GCTCACCATCATTGGGCTCCGTGGCCCCAATG-3′
[SEQ ID NO: 146]
5′-CATTGGGGCCACGGAGCCCAATGATGGTGAGC-3′

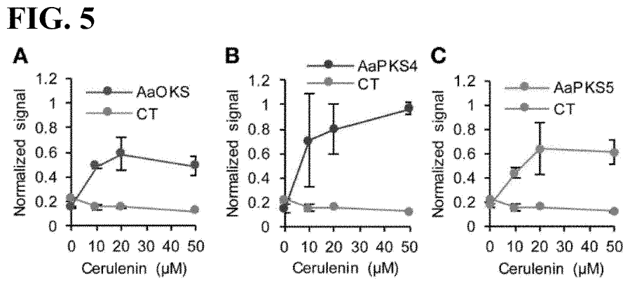

When an E. coli BL21(DE3) strain containing each of the constructed plasmids was cultured, change in the color of the strains expressing AaOKS, AaPKS4 and AaPKS5 could be observed. Thus, polyketide synthases that contributed to the color changes were analyzed by LC-MS analysis (FIG. 4A). As noted above, malonyl-CoA experiments using cerulenin were performed, and as a result, it was demonstrated that the signal generated from each strain increased as the intracellular malonyl-CoA concentration increased (FIGS. 4 and 5). Here, the signal indicates the absorbance at 300 nm of the culture supernatant (FIG. 4C). Therefore, in the present invention, it was demonstrated that not only the RppA enzyme, all other type III polyketide synthases capable of producing colored polyketide-based compounds from malonyl-CoA, can be used as malonyl-CoA biosensors.

In the following Examples, additional experiments were performed using RppA among the above-described type III polyketide synthases.

1.3. Examination of Scalability to Other Industrially Useful Strains

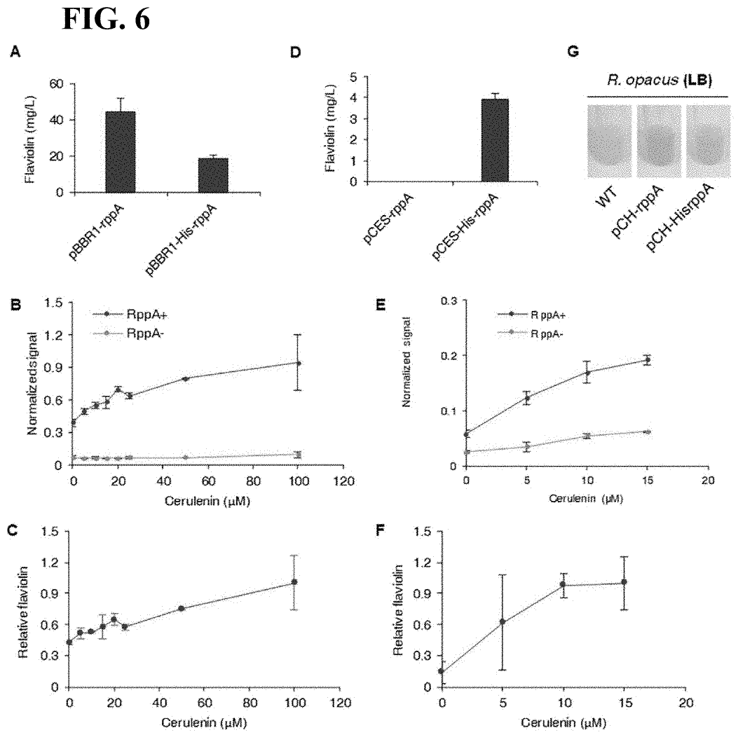

After the applicability in E. coli was demonstrated in the above-described Examples, the scalability of this RppA malonyl-CoA biosensor to other industrially useful strains was examined. Examples of strains which are used in this examination include, in addition to E. coli, Pseudomonas putida which is a representative Gram-negative bacterium, and Corynebacterium glutamicum and Rhodococcus opacus, which are representative gram-positive bacteria. First, a plasmid for producing flaviolin in a P. putida strain was constructed using pBBR1MCS2 as a base plasmid (Kovach M E, et al. (1995), Gene 166:175-176). First, the pBBR1MCS2 plasmid was linearized using the primers of SEQ ID NO: 12 and SEQ ID NO: 13. Then, an rppA expression cassette containing the tac promoter was amplified by PCR using the pTac-Sgr_rppA plasmid as a template and the primers of SEQ ID NO: 14 and SEQ ID NO: 15. The obtained two DNA fragments were assembled using Gibson assembly, thereby constructing a pBBR1-rppA plasmid. Furthermore, in order to examine the effect of the N-terminal poly His-tag on enzyme expression and on flaviolin production, pTac-His-Sgr_rppA was first constructed. At this time, a pTacCDFS plasmid, linearized by PCR using the primers of SEQ ID NO: 9 and SEQ ID NO: 10, and a His-tagged rppA gene amplified by PCR from the genomic DNA of S. griseus using the primers of SEQ ID NO: 16 and SEQ ID NO: 8 were assembled into a single plasmid (pTac-His-Sgr_rppA) using Gibson assembly. The constructed plasmid was amplified again by PCR using the primers of SEQ ID NO: 14 and SEQ ID NO: 15, and assembled with the linearized pBBR1MCS2 plasmid using Gibson assembly, thereby constructing a pBBR1-His-rppA plasmid. At this time, each of pBBR1-rppA and pBBR1-His-rppA plasmids was transformed into a P. putida strain, and then each of the resulting strains was inoculated into a test tube containing 5 mL of LB medium, and then cultured at 30° C. and 220 rpm for 18 hours. Next, 0.8 g/L MgSO₄.7H₂O, 10 g/L glucose, and an antibiotic were added to 5 mL LB, and then subculture was performed. After culture under the same conditions for 25 hours, sampling was performed. As a result, a larger amount of flaviolin was produced in the strain containing the pBBR1-rppA plasmid (44.7 mg/L; FIG. 6A). Accordingly, using the screened P. putida pBBR1-rppA strain, characterization of the malonyl-CoA biosensor was performed using cerulenin in the same manner as described in Example 1.2 above. As a result, it could be observed that the normalized signal and the normalized production of flaviolin also increased as the cerulenin concentration in the corresponding strain increased (FIGS. 6B and 6C). Here, the subculture was performed in 2 mL of MR-MOPS medium supplemented with 20 g/L glucose, 25 μg/mL kanamycin, 0.5 mM IPTG and a suitable concentration of cerulenin, and the composition of the MR-MOPS medium (pH 7.0) contained, per liter, the following components: 6.67 g KH₂PO₄, 4 g (NH₄)₂HPO₄, 0.8 g citric acid, 5 mL trace metal solution, 20.09 g MOPS (3-(N-morpholino)propanesulfonic acid), 0.8 g/L MgSO₄.7H₂O. As a result, it was demonstrated that the RppA malonyl-CoA biosensor successfully acts in P. putida.

[SEQ ID NO: 12]
5′-CGAACTCAGAAGTGAAACGCTGCCTAATGAGTGAGCTAAC-3′
[SEQ ID NO: 13]
5′-CATTGGTGCACCGTGCAGTCAAAATTCGCGTTAAATTTTTG-3′
[SEQ ID NO: 14]
5′-GACTGCACGGTGCACCAATG-3′
[SEQ ID NO: 15]
5′-GCGTTTCACTTCTGAGTTCG-3′
[SEQ ID NO: 16]
5′-CAATTTCACACAGGAAACAGAATGCACCATCACCATCACCATGCGACC
CTGTGCCGACC-3′

In addition, in order to apply this biosensor to a C. glutamicum strain, an rppA expression plasmid was constructed using pCES-H36 as a base plasmid (Yim S S, An S J, Kang M, Lee J, Jeong K J (2013), Biotechnol Bioeng 110:2959-2969). To this end, the corresponding plasmid was linearized using a pCES-H36-GFP plasmid as a template and the primers of SEQ ID NO: and SEQ ID NO: 18. A tac promoter-based rppA expression cassette was amplified by PCR using pTac-Sgr_rppA as a template and the primers of SEQ ID NO: 19 and SEQ ID NO: 20, and an N-terminal His-tagged rppA expression cassette based on the tac promoter was amplified by PCR using pTac-His-Sgr_rppA as a template and the primers of SEQ ID NO: 21 and SEQ ID NO: 20. The two different rppA expression cassettes were assembled with the above-described linearized pCES-H36 plasmid using Gibson assembly, thereby constructing pCES-rppA and pCES-His-rppA plasmids. The constructed plasmids were transformed into a C. glutamicum strain, and then inoculated into 5 mL of LB medium. Each of the resulting strains was cultured in an incubator at 30° C. and 220 rpm for 18 hours, and then subcultured in 5 mL of LB medium for 48 hours, after which sampling was performed. At this time, 3.9 mg/L of flaviolin was produced only in the strain containing the N-terminal His-tagged rppA expression cassette (pCES-His-rppA) (FIG. 6D). Accordingly, using the selected C. glutamicum pCES-His-rppA strain, characterization of the malonyl-CoA biosensor was performed using cerulenin in the same manner as described in Example 1.2 above. As a result, it could be observed that the normalized signal and the normalized production of flaviolin also increased as the cerulenin concentration in the corresponding strain increased (FIGS. 6E and 6F).

[SEQ ID NO: 17]
5′-CCATTATAATTAGGCCTCGG-3′
[SEQ ID NO: 18]
5′-CCATGCTACTCCTACCAACC-3′
[SEQ ID NO: 19]
5′-GGTTGGTAGGAGTAGCATGGGATCCATGGCGACCCTGTGCCGACC-3′
[SEQ ID NO: 20]
5′-CCGAGGCCTAATTATAATGGATTAGCCGGACAGCGCAACGC-3′
[SEQ ID NO: 21]
5′-GGTTGGTAGGAGTAGCATGGGATCCATGCACCATCACCATCACCATG
C-3′

In another example, in order to apply this biosensor to an R. opacus strain, an rppA expression plasmid was constructed using pCH as a base plasmid. An acetamide-inducible promoter (G. Roberts et al., FEMS Microbiol. Lett. 222:131-136, 2003) was used, which was amplified by PCR using the primers of SEQ ID NO: 22 and SEQ ID NO: 23. In addition, an rppA expression cassette was amplified by PCR from the genomic DNA of S. griseus using the primers of SEQ ID NO: 24 and SEQ ID NO: 25, and an rppA expression cassette for additional expression of the N-terminal His-tag was amplified by PCR using the primer of SEQ ID NO: 26 and SEQ ID NO: 25. Each rppA expression cassette, the acetamide-inducible promoter DNA fragment, and the pCH plasmid linearized by PstI were assembled together using Gibson assembly, thereby constructing two vectors (pCH-rppA and pCH-His-rppA). Each of the constructed plasmids was transformed into an R. opacus strain, and then inoculated into 5 mL of LB medium. Each of the resulting strains was cultured in an incubator at 30° C. and 220 rpm for 18 hours, and then subcultured in 5 mL of LB medium for 48 hours, after which sampling was performed. It could be seen that red-colored flaviolin was produced only in the strain containing pCH-rppA (FIG. 6G).

[SEQ ID NO: 22]
5′-CTTGATCAGCTTGCATGCCTGCAGAAGCTITCTAGCAGAAATAATTC-
3′
[SEQ ID NO: 23]
5′-GTGCATGTGGACTCCCTTTCTCTTATC-3′
[SEQ ID NO: 24]
5′-GATAAGAGAAAGGGAGTCCACATGGCGACCCTGTGCCGACC-3′
[SEQ ID NO: 25]
5′-GGATCCTCTAGAGTCGACCTGCAGATTAGCCGGACAGCGCAACGC-3′
[SEQ ID NO: 26]
5′-GATAAGAGAAAGGGAGTCCACATGCACCATCACCATCACCATGC-3′

### Example 2. High-Throughput Screening of Strain Having Increased Malonyl-CoA Producing Ability by Use of RppA Biosensor

2.1. High-Throughput Screening of Strain Having Increased Malonyl-CoA Producing Ability by Introduction of E. coli Genome-Scale Synthetic Control sRNA Library

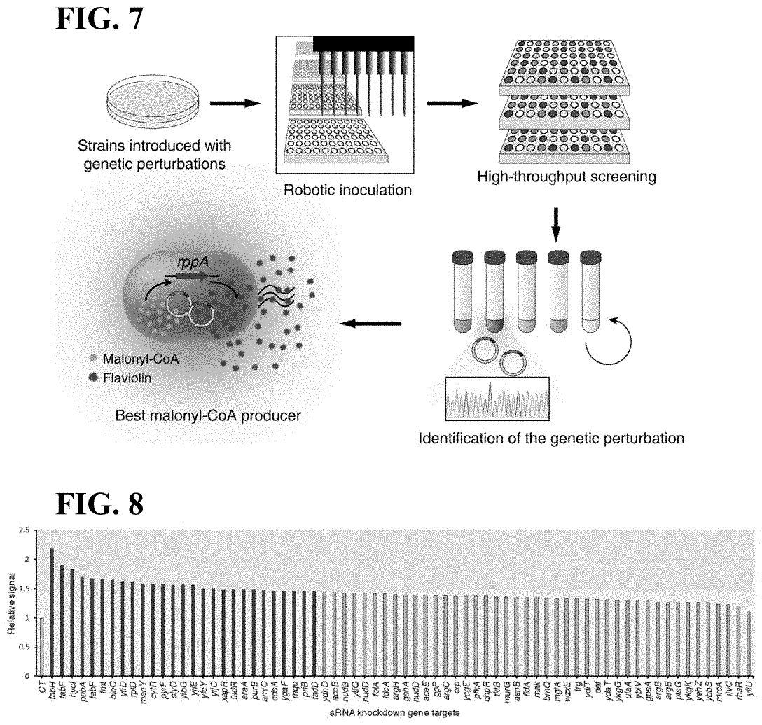

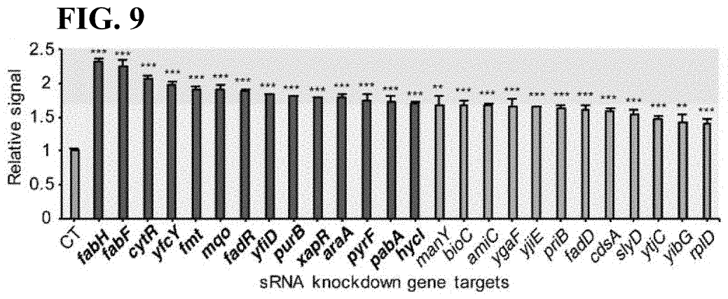

After construction of the RppA malonyl-CoA biosensor, confirmation of successful operation thereof, and demonstration of the applicability and scalability of the biosensor, synthetic control sRNA technology developed by the present inventors was introduced in order to screen a strain having increased malonyl-CoA producing ability by the use of the biosensor (KR 10-1575587, U.S. Pat. No. 9,388,417, EP 13735942.8, CN 201380012767.X, KR 10-1690780, KR 10-1750855, U.S. Ser. No. 15/317,939, CN 201480081132.X; Na D, et al. (2013), Nat Biotechnol 31:170-174; Yoo S M, Na D, Lee S Y (2013), Nat Protoc 8:1694-1707). In addition, in order to include all major genes in E. coli, a previously constructed E. coli genome-scale synthetic-control sRNA library (including 1,858 genes in E. coli K-12 W3110 strain) was introduced and knocked down at the gene level, and knockdown gene targets effective for increased production of malonyl-CoA were screened. Thus, the E. coli genome-scale synthetic-control sRNA library was introduced into an E. coli BL21(DE3) strain containing a pTac-5′UTR_Sgr_rppA plasmid, and then high-throughput screening was performed (FIG. 7). In order to cover all 1,858 sRNAs, 11,488 colonies having a size equal to larger than 6 times the library size were selected from the obtained colonies, and then a robotic automatic high-throughput screening system was used. To this end, a K3 colony picker (KBiosystems, Basildon, UK) was used, and each colony was inoculated into LB medium (supplemented with antibiotic and 0.2 mM IPTG) in 96-well microplates by a robot (Korea Research Institute of Bioscience and Biotechnology (KRIBB); Jeongeup, Republic of Korea). The K3 colony picker illuminates and images the agar plates. Then, the intrinsic program recognizes and locates colonies and instructs a robot arm tipped with pins to pick the selected colonies. The inoculated cells were cultured in a HT-MegaGrow incubator (Bioneer, Republic of Korea) at 30° C. and 500 rpm for 24 hours. After culture, 231 strains with relatively strong signals were selected. The selected strains were cultured in 14 mL disposable Falcon round-bottom tubes containing 3 mL of LB medium supplemented with antibiotics and 0.2 mM IPTG at 30° C. and 250 rpm for 24 hours. Among them, 70 strains showed high signals than the control strain (biosensor strain containing no sRNA), and sRNAs contained in these strains were sequenced (FIG. 8). At this time, three sRNAs were observed twice (argB, fabF, nudD). In addition, among them, 26 strains showed an increase in signal of 45% or more compared to the control strain. Accordingly, sRNA vectors corresponding to the 26 strains were transformed back to the original sensor strain (FIG. 9), and cultured again in 3 mL of LB as described above. As a result, among them, 14 sRNAs showing an increase in signal of more than 70% compared to the control strain were finally selected. The 14 selected knockdown gene targets for increased malonyl-CoA production are listed in Table 3 below.

TABLE 3
List of 14 finally selected knockdown target genes

| No. | Target gene | Protein function | Essentiality^{‡} |
|---|---|---|---|
| 1 | fabH | 3-oxoacyl-[acyl-carrier- | NE |
| | | protein] synthase III | |
| 2 | fabF | 3-oxoacyl-[acyl-carrier- | NE |
| | | protein] synthase II | |
| 3 | cytR | LacI family transcriptional | NE |
| | | regulator, repressor for | |
| | | deo operon, udp, cdd, tsx, | |
| | | nupC and nupG | |
| 4 | yfcY | beta-ketoacyl-CoA thiolase | NE |
| 5 | fmt | 10- | ND |
| | | formyltetrahydrofdate: L- | |
| | | methionyl-tRNA(fMet)N- | |
| | | formyltransferase | |
| 6 | mqo | malate dehydrogenase, | ND |
| | | FAD/NAD(P)-binding domain | |
| 7 | fadR | GntR family transcriptional | NE |
| | | regulator, negative | |
| | | regulator for fad regulon | |
| | | and positive regulator of | |
| | | fabA | |
| 8 | yfiD | pyruvate formate lyase | NE |
| | | subunit | |
| 9 | purB | adenylosuccinate lyase | NE |
| 10 | xapR | transcriptional activator | NE |
| | | of xapAB | |
| 11 | araA | L-arabinose isomerase | ND |
| 12 | pyrF | orotidine-5′-phosphate | E |
| | | decarboxylase | |
| 13 | pabA | aminodeoxychorismate | E |
| | | synthase, subunit II | |
| 14 | hycI | protease involved in | NE |
| | | processing C-terminal end | |
| | | of HycE | |

2.2. Confirmation of Overexpression Gene Targets for Increased Malonyl-CoA Production, Identified Using FVSEOF Algorithm, by RppA Biosensor

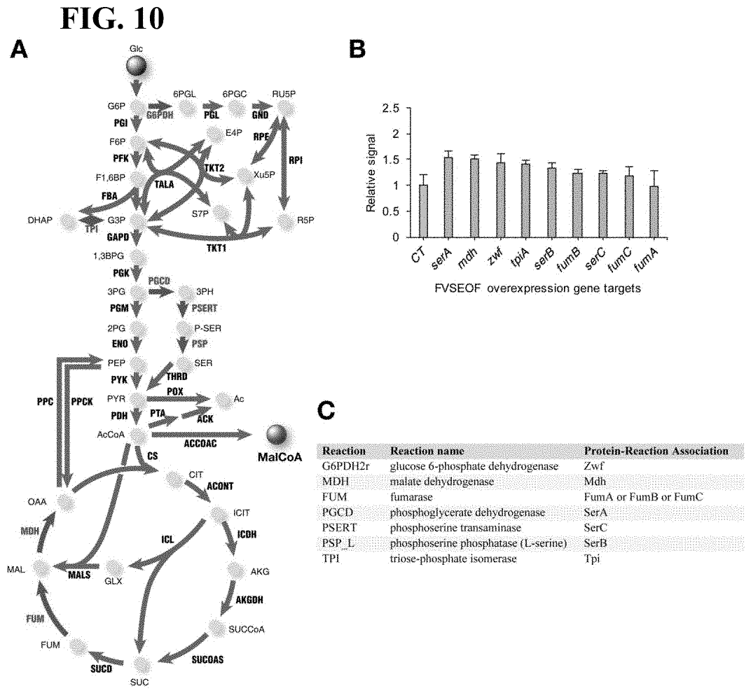

Not only knockdown gene targets, but also overexpression gene targets were demonstrated by the RppA biosensor. At this time, gene targets identified using an FVSEOF algorithm were used (Park J M, et al. (2012), BMC Syst Biol 6:106). In silico analysis was performed using the E. coli genome-scale metabolic model iJO1366. (Orth J D, et al. (2011), Mol Syst Biol 7:535). Thus identified 9 gene targets are as follows: zwf, mdh, fumA, fumB, fumC, serA, serB, serC, and tpiA (FIGS. 10A and 10C). Each gene target was amplified in E. coli BL21(DE3), and inserted into a pTrc99A plasmid under the trc promoter, and transformed into a BL21(DE3) pTac-5′UTR-Sgr_rppA strain. The constructed sensor strains were cultured in test tubes in the same manner as described in Example 2.1 above, and at this time, signals were measured. As a result, increased signals compared to the control were observed in 8 gene targets (serA, mdh, zwf, tpiA, serB, fumB, serC, and fumC) among 9 gene targets (FIG. 10B).

### Example 3. Increased Production of Useful Products Using Knockdown Gene Targets Screened Using RppA Biosensor

Flask culture conditions used in this Example and the following Examples are as follows unless otherwise specified. A colony was inoculated into a test tube containing 10 mL of LB medium, and cultured in an incubator at 37° C. and 200 rpm. 1 mL of the cell culture was inoculated into a baffled flask containing 50 mL of modified R/2 medium. When strains which have been grown at 37° C. and 200 rpm reached an OD₆₀₀ value of 0.8, the strains were treated with 0.5 mM IPTG to induce production, and were cultured at 30° C. and 200 rpm for 48 hours. As a carbon source, glycerol or glucose was added.

3.1. Increased Production of 6-Methylsalicylic Acid Using Selected Knockdown Gene Targets

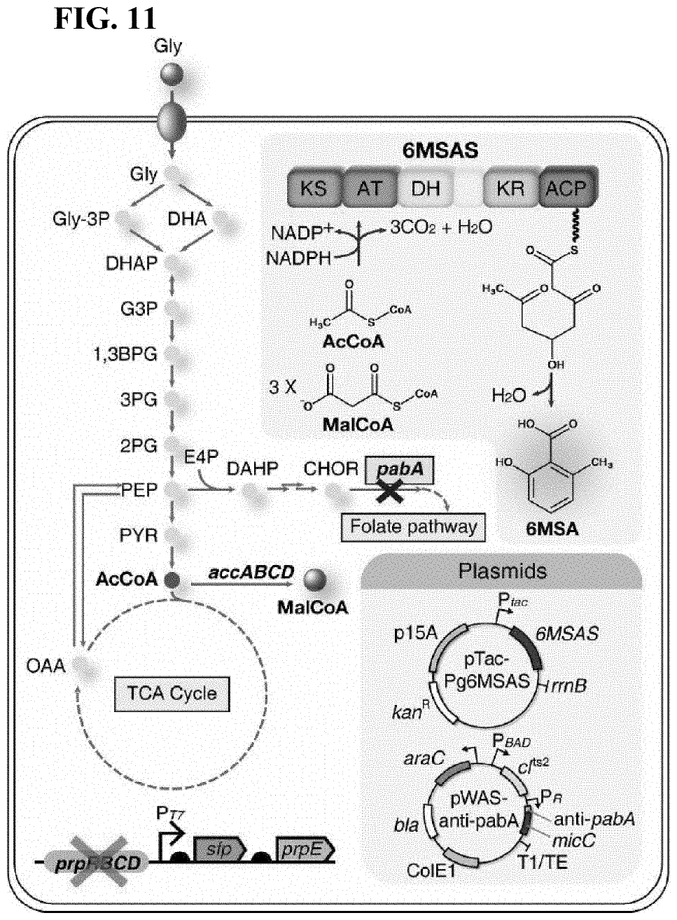

In the next step, the knockdown gene targets, selected in the above Example and enabling increased malonyl-CoA production, were applied for production of useful products in order to demonstrate that the biosensor of the present invention could successfully screen effective knockdown gene targets. Accordingly, as the first product, 6-methylsalicylic acid (6MSA) reported to be produced from a Penicilium griseofulvum (or Penicilium patulum) strain was attempted to be produced from genetically engineered E. coli. 6-Methylsalicylic acid possesses antibiotic and antifungal activities (Dimroth P, Ringelmann E, Lynen F (1976), Eur J Biochem 68:591-596), and is produced from one molecule of acetyl-CoA and three molecules of malonyl-CoA by a 6-methylsalicylic acid synthase (6MSAS), type I iterative polyketide synthase, found in fungi (FIG. 11).

The sequence of 6-methylsalicylic acid synthase (6MSAS) that was used in the present invention is as follows:

6-methylsalicylic acid synthase (6MSAS) [SEQ
ID NO: 123]:
mhsaatstyp sgktspapvg tpgteyseye fsndvavvgm
acrvaggnhn pellwqslls qksamgeipp mrwepyyrrd
arnekflknt tsrgyfldrl edfdcqffgi spkeaeqmdp
qqrvslevas ealedagipa kslsgsdtav fwgvnsddys
klvledlpnv eawmgigtay cgvpnrisyh lnlmgpstav
daacasslva ihhgvqairl geskvaivgg vnalcgpglt
rvldkagais sdgscksfdd dahgyargeg agalvlkslh
ralldhdnvl avikgsavcq dgktngimap nsvaqqlaan
nalsaanidp htvryveaha tstplgdpte isaiasvyga
drpaddpcyi gsikpnighl eagagvmgfi kavlaiqkgv
lppqanltkl nsridwktag vkvvqeatpw pesdpirrag
vcsygyggtv shavieefsp ilqpdplgng aysgpgllll
sgpqekrlal qaktlrdwmt aegkdhnlsd ilttlatrrd
hhdyraalvv ddyrdaeqvl qslangvdht fttqsrvlgs
diskdvvwvf sghgaqwpdm gkqlihnpvf faaiqpldel
iqaeiglspi ellrtgdfes sdrvqiltyv mqiglsallq
sngitpqavi ghsvgeiaas vvagalspae galivtrral
lyrqvmgkgg milvnlpsae teeilgsrsd lvvaidssps
scvvagdkel vaetaealka rgvktftvks diafhsptln
glvdplrdvl aetlspvspn vklystalad prgqdlrdve
ywagnmvnry rltsavkaav edgyrlflev sthpvvshsi
netlmdagme dfaviptllr kkptekhilh siaqlhcrga
evnwaaqmpg rwatgvpttt wmhkpiwrki etaplhtglt
hdvekhtllg qripvpgtdt yvyttrldnd tkpfpgshpl
hgteivpaag lintflkgtg gqmlqnvvlr vpvainaprs
vqvvvqqdqv kvvsrlipse psqldddasw vthttaywdr
kvagsedrid faavksrlvt kladnfsidy ldkvgvsamg
fpwavtehyr ndkemlarvd vnpaisgdap lpwdssswap
vldaatsvgs tifptpalrm paqiervevf tsqdppkisw
lyvqeasdsv ptshvsvvse agevlakfta mrfseiegtp
gvsgsmeslv hqiawppatp aeeplsietv ilvspdattr
alyaaslptr vnsfqfsstq effsnasslp lekgtvvtyi
pgevaslaev paasesftwn llelikftvn gslpikvftl
tanigegqtp talaqsplyg larviasehp dlgtlidvee
pviplstmry iqgadiirin dgiartsrfr slprnkllpa
segprllprp egtylitggl gvlglevadf lvekgarrll
lisrralppr rtwdqvsedl qptiakirll esrgasvhvl
plditkpdav eqlttaldrl slpsvqgvvh aagvldnelv
mqttrdafnr vlapkiagal alhevfppks vdffvmfssc
gnlvgftgqa sygsgnafld tlathrarlg daaysfqwts
wrglgmgast dfinaelesk gitdvtrdea faawqhlaky
dmdhgvvirs rafedgepip vsilndiavr rvgtvsntsp
aaagssdavp tsgpelkayl dekirgcvak vlqmtaedvd
skaaladlgv dsvmtvtlrr qlqltlkiav pptltwshpt
vshlavwfae klak

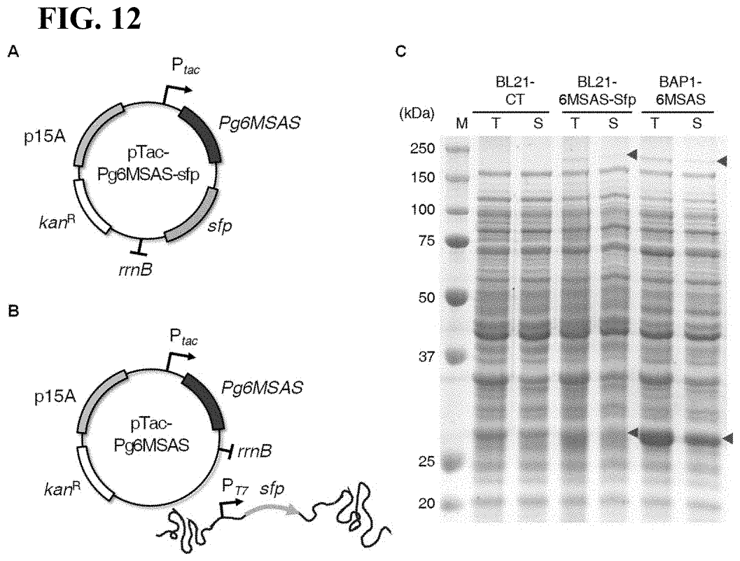

In order to construct an E. coli strain capable of producing 6-methylsalicylic acid, a pTac15K plasmid was first linearized by inverse PCR using the primers of SEQ ID NO: 27/SEQ ID NO: 28, and then a Pg6MSAS gene encoding 6-methylsalicylic acid synthase was amplified using the genomic DNA of P. griseofulvum as a template and sequentially using the primers of SEQ ID NO: 29 and SEQ ID NO: 30, and SEQ ID NO: 31 and SEQ ID NO: 30. The two DNA fragments were assembled using Gibson assembly, thereby constructing a pTac-Pg6MSAS plasmid (FIG. 12B). The 6MSAS enzyme contains an acyl-carrier protein domain at its terminus, and 4′-phosphopantetheinyl transferase (Sfp) is required to activate this domain.

The sequence of 4′-phosphopantetheinyl transferase (Sfp) that was used in the present invention is as follows:

4′-phosphopantetheinyl transferase (Sfp)
[SEQ ID NO: 124]:
mkiygiymdr plsqeenerf msfispekre kcrrfyhked
ahrtllgdvl vrsvisrqyq ldksdirfst qeygkpcipd
lpdahfnish sgrwvicafd sqpigidiek tkpisleiak
rffskteysd llakdkdeqt dyfyhlwsmk esfikqegkg
lslpldsfsv rlhqdgqvsi elpdshspcy iktyevdpgy
kmavcavhpd fpeditmvsy eell

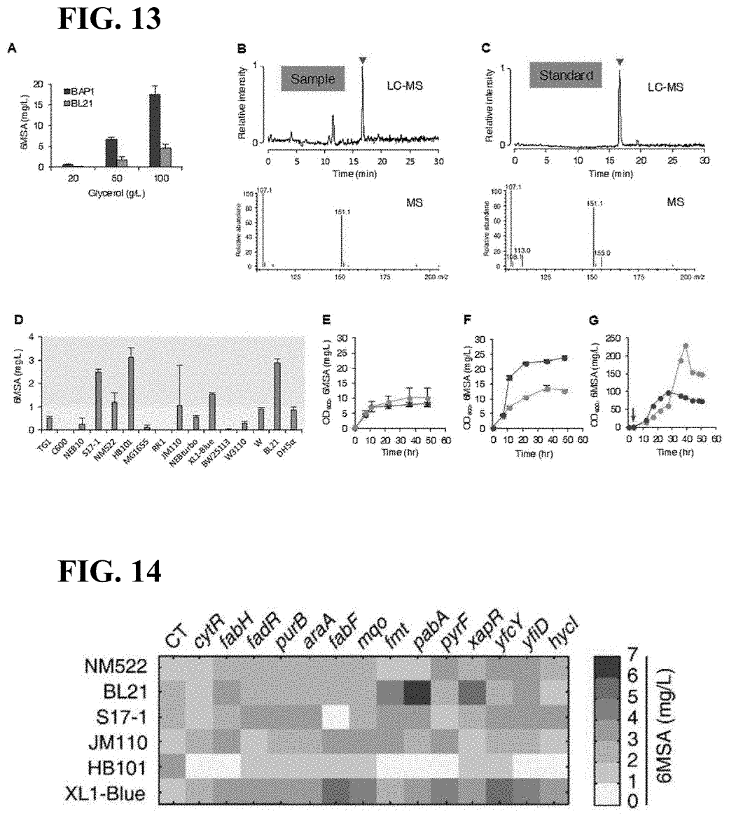

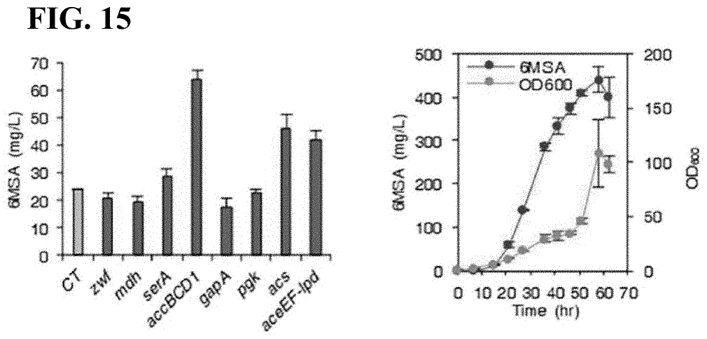

sfp gene fragment encoding 4′-phosphopantetheinyl transferase was amplified by PCR from the genomic DNA of Bacillus subtilis using the primers of SEQ ID NO: 32 and SEQ ID NO: 33, and inserted into a pTac15K plasmid at the EcoRI site. The resulting plasmid was amplified again by PCR using the primers of SEQ ID NO: 34 and SEQ ID NO: 35, and then inserted into a pTac-Pg6MSAS plasmid at the SphI site, thereby constructing a pTac-Pg6MSAS-sfp plasmid (FIG. 12A). The constructed plasmid was transformed into an E. coli BL21(DE3) strain, and enzyme expression was checked by SDS-PAGE (FIG. 12C). Then, flask culture of the strain was performed in modified R/2 medium in the presence of different concentrations of glucose or glycerol, and as a result, it could be seen that when 100 g/L of glycerol was added, 4.7 mg/L of 6-methylsalicylic acid was produced (FIG. 13A). The authenticity of the produced 6-methylsalicylic acid was confirmed by LC-MS (FIGS. 13B and 13C). Before introduction of the sRNAs selected in the above Example, in order to examine the production of 6-methylsalicylic acid in different E. coli strains, the pTac-Pg6MSAS-sfp plasmid was introduced into the 16 E. coli strains shown in Table 1 above, and then test tube-scale culture of each of the strains was performed in 3 mL of modified R/2 medium. Among these strains, 6 strains (NM522, BL21, S17-1, JM110, HB101, and XL1-Blue) producing more than 1 mg/L of 6MSA were selected (FIG. 13D). Then, 14 selected sRNAs were introduced into each of the selected strains, thereby constructing a total of 84 strains, and test tube-scale of each of the constructed strains was performed. As a result, the pabA-knockdown BL21(DE3) strain produced the largest amount of 6-methylsalicylic acid (6.1 mg/L; FIG. 14). This strain was further subjected to flask culture (50 mL) under the same medium conditions, and as a result, it produced 8.0 mg/L of 6-methylsalicylic acid (FIG. 13E).

[SEQ ID NO: 27]
5′-GCTGAGAAGCTTGCCAAATAATGGATCCTCTAGAGTCGACCTG-3′
[SEQ ID NO: 28]
5′-CTGGGGATGTTTTCCCAGAGGGGTATGTAGAAGTTGCAGCGGAATGCA
TGAATTCTGTTTCCTGTGTGAAATTG-3′
[SEQ ID NO: 29]
5′-ACAGTGAATATGAATTCTCCAACG-3′
[SEQ ID NO: 30]
5′-ATTATTTGGCAAGCTTCTCAGC-3′
[SEQ ID NO: 31]
5′-CTCTGGGAAAACATCCCCAGCACCAGTCGGAACCCCTGGGACTGAGT
ACAGTGAATATGAATTCTCCAACG-3′
[SEQ ID NO: 32]
5′-TAATAAGAATTCATGAAGATTTACGGAATTTATATG-3′
[SEQ ID NO: 33]
5′-TTATTAGAATTCTTATAAAAGCTCTTCGTACGAG-3′
[SEQ ID NO: 34]
5′-CTAGAGTCGACCTGCAGGCATGCCACTCCCGTTCTGGATAATG-3′
[SEQ ID NO: 35]
5′-CAAAACAGCCAAGCTTGCATGC-3′

In order to further optimize the 6-methylsalicylic acid producing strain, only the Pg6MSAS gene having a long length (5.3 kb) was expressed with a plasmid (pTac-Pg6MSAS), and sfp was expressed using the strain BAP1 (Pfeifer B A, Admiraal S J, Gramajo H, Cane D E, Khosla C (2001), Science 291:1790-1792), which is a strain that sfp is inserted into the genome of the BL21(DE3) strain. The results of flask culture showed an increased 6-methylsalicylic acid production of 17.6 mg/L, and from the results of SDS-PAGE analysis, this was believed to be because of improved 4′-phosphopantetheinyl transferase expression and the reduced proportion of inactivated 6-methylsalicylic acid synthase (FIG. 12C). Accordingly, when a pabA-knockdown sRNA was introduced into an E. coli BAP1 pTac-Pg6MSAS strain, 6-methylsalicylic acid could be produced in an amount of 23.9 mg/L, which corresponding to an increase of about 35.8% (FIG. 13F). When fed-batch fermentation of this strain was performed, an increased 6-methylsalicylic acid concentration of 97.8 mg/L could be obtained after about 27 hours (FIG. 13G). The fed-batch fermentation was performed in 1.9 L of modified R/2 medium in a 6.6-L fermenter (BioFlo320, Eppendofr, Germany) in the presence of 50 g/L of glycerol as an initial carbon source. The colony was inoculated into a test tube containing 10 mL of LB medium, and then cultured at 37° C. and 200 rpm for one day. Next, the cells were inoculated into two baffled flasks, each containing 50 mL of modified R/2 medium, and at this time, 50 g/L of glycerol was used as a carbon source. Flask culture was performed for about 9 hours until the OD₆₀₀ value reached about 2, and then the cells were inoculated into a fermenter. The culture pH was maintained at 6.8 by 28% (v/v) ammonia solution, and the dissolved oxygen (DO) concentration was controlled at 40% of air saturation by supplying air at 2 L/min and automatically controlling the agitation speed as 1000 rpm and by increasing the oxygen flow rate. The pH-stat feeding strategy was employed in order to supply nutrients. When the pH becomes higher than 6.86, the feeding solution was automatically added. The feed solution contained, per liter, the following components: 800 g glycerol, 6 mL trace metal solution and 12 g MgSO₄.7H₂O. Heterologous protein expression was induced with 0.5 mM IPTG when the OD₆₀₀ value after inoculation reached 2 to 3.

In order to further increase the production of 6-methylsalicylic acid, it was attempted to increase the expression of the previously selected three FVSEOF gene targets (P<0.05; zwf, mdh, serA; FIG. 10) and the following five enzymes: C. glutamicum acetyl-CoA carboxylase (AccBC and AccD1), E. coli glyceraldehyde 3-phosphate dehydrogenase (GapA), E. coli phosphoglycerate kinase (Pgk), E. coli acetyl-CoA synthetase (Acs), and E. coli pyruvate dehydrogenase (PDH: AceEF and Lpd). At this time, the corresponding gene fragments were inserted and cloned into a pBBR1TaC plasmid. The pBBR1TaC plasmid is a tac promoter-based expression plasmid constructed from pBBR1MCS. For construction of pBBR1TaC, pBBR1MCS was first amplified by inverse PCR using the primers of SEQ ID NO: 147 and SEQ ID NO: 148, and a DNA fragment composed of tac promoter, MCS and rrnBT1T2 terminator was amplified using pTac15K as a template and the primers of SEQ ID NO: 149 and SEQ ID NO: 150, and cloned into the plasmid using Gibson assembly. Next, in order to transfer genes from the pTrc99A-zwf, pTrc99A-mdh and pTrc99A-serA plasmids constructed in Example 2.2 above to pBBR1TaC, the following experiment was performed. First, zwf, mdh and serA genes were each amplified using the primers of SEQ ID NO: 151 and SEQ ID NO: 152, and the pBBR1TaC plasmid was amplified using the primers of SEQ ID NO: 75 and SEQ ID NO: 76, and then subjected to Gibson assembly, thereby constructing pBBR1-zwf, pBBR1-mdh and pBBR1-serA plasmids. C. glutamicum acetyl-CoA carboxylase is composed of the alpha subunit AccBC and the beta subunit AccD1, and the corresponding genes were each cloned into pBBR1TaC, thereby constructing pBBR1-accBC and pBBR1-accD1. At this time, the accBC gene was amplified using the primers of SEQ ID NO: 153 and SEQ ID NO: 154, and the accD1 gene was amplified using the primers of SEQ ID NO: 155 and SEQ ID NO: 156. The two gene fragments were each inserted by Gibson assembly into the pBBR1TaC plasmid amplified using the primers of SEQ ID NO: 9 and SEQ ID NO: 10. Using pBBR1-accD1 as a template among the two constructed plasmids, a DNA fragment containing tac promoter and accD1 was amplified using the primers of SEQ ID NO: 157 and SEQ ID NO: 158, and then inserted by Gibson assembly into a pBBR1-accBC plasmid digested by HindIII restriction enzyme, thereby constructing a pBBR1-accBCD1 plasmid. E. coli pyruvate dehydrogenase (PDH) is composed of subunits E1 (AceE), E2 (AceF) and E3 (Lpd). First, an aceEF gene fragment and an lpd gene fragment were cloned into a pBBR1TaC plasmid, thereby constructing pBBR1-aceEF and pBBR1-lpd plasmids. The gene fragment aceEF was amplified using the primers of SEQ ID NO: 159 and SEQ ID NO: 160, and lpd was amplified using the primers of SEQ ID NO: 161 and SEQ ID NO: 162. The two gene fragments were each inserted by Gibson assembly into the linearized pBBR1TaC plasmid used for construction of the pBBR1-accBC and pBBR1-accD1 plasmids. Using pBBR1-lpd as a template among the two constructed plasmids, a gene fragment comprising tac promoter and lpd was amplified using the primers of SEQ ID NO: 163 and SEQ ID NO: 164, and then inserted into a pBBR1-aceEF plasmid digested by SalI restriction enzyme, thereby constructing a pBBR1-aceEF-lpd plasmid. For construction of pBBR1-gapA, pBBR1-pgk and pBBR1-acs plasmids containing E. coli glyceraldehyde 3-phosphate dehydrogenase (GapA), E. coli phosphoglycerate kinase (Pgk) and E. coli acetyl-CoA synthetase (Acs), respectively, gapA, pgk and acs gene fragments were amplified by PCR using the primers of SEQ ID NO: 165 and SEQ ID NO: 166, SEQ ID NO: 167 and SEQ ID NO: 168, and SEQ ID NO: 169 and SEQ ID NO: 170, and then each inserted by Gibson assembly into the linearized pBBR1TaC plasmid.

[SEQ ID NO: 147]
5′-GACGGATGGCCTTTTACTAGTGCCTGGGGTGCCTAATGAG-3′
[SEQ ID NO: 148]
CGATGATTAATTGTCAACTGCTACGCCTGAATAAGTGATAATAAG-3′
[SEQ ID NO: 149]
5′-GTTGACAATTAATCATCGGCTC-3′
[SEQ ID NO: 150]
5′-ACTAGTAAAAGGCCATCCGTCAGGATG-3′
[SEQ ID NO: 151]
5′-GTTGACAATTAATCATCGGC-3′
[SEQ ID NO: 152]
5′-CGTTTCACTTCTGAGTTCGG-3′
[SEQ ID NO: 153]
5′-CAATTTCACACAGGAAACAGAATTCGTGTCAGTCGAGACTAGGAAG-
3′
[SEQ ID NO: 154]
5′-CTCTAGAGGATCCCCGGGTACCATTACTTGATCTCGAGGAGAAC-3′
[SEQ ID NO: 155]
5′-CAATTTCACACAGGAAACAGAATTCATGACCATTTCCTCACCTTTG-
3′
[SEQ ID NO: 156]
5′-CTCTAGAGGATCCCCGGGTACCATTACAGTGGCATGTTGCCGTG-3′
[SEQ ID NO: 157]
5′-GAGTCGACCTGCAGGCATGCATTGACAATTAATCATCGGCTCG-3′
[SEQ ID NO: 158]
5′-CTCATCCGCCAAAACAGCCAAGCTT-3′
[SEQ ID NO: 159]
5′-CAATTTCACACAGGAAACAGAATTCATGTCAGAACGTTTCCCAAATG-
3′
[SEQ ID NO: 160]
5′-CTCTAGAGGATCCCCGGGTACCATTACATCACCAGACGGCGAATG-3′
[SEQ ID NO: 161]
5′-CAATTTCACACAGGAAACAGAATTCATGAGTACTGAAATCAAAACTC-
3′
[SEQ ID NO: 162]
5′-CTCTAGAGGATCCCCGGGTACCATTACTTCTTCTTCGCTTTCGG-3′
[SEQ ID NO: 163]
5′-GTACCCGGGGATCCTCTAGAGTTGACAATTAATCATCGGCTCG-3′
[SEQ ID NO: 164]
5′-CAAGCTTGCATGCCTGCAGGTCGAC-3′
[SEQ ID NO: 165]
5′-CAATTTCACACAGGAAACAGAATTCATGACTATCAAAGTAGGTATCAA
C-3′
[SEQ ID NO: 166]
5′-CTCTAGAGGATCCCCGGGTACCATTATTTGGAGATGTGAGCGATC-3′
[SEQ ID NO: 167]
5′-CAATTTCACACAGGAAACAGAATTCATGTCTGTAATTAAGATGACCGA
TC-3′
[SEQ ID NO: 168]
5′-CTCTAGAGGATCCCCGGGTACCATTACTTCTTAGCGCGCTCTTCG-3′
[SEQ ID NO: 169]
5′-CAATTTCACACAGGAAACAGAATTCATGAGCCAAATTCACAAACAC-
3′
[SEQ ID NO: 170]
5′-CTCTAGAGGATCCCCGGGTACCATTACGATGGCATCGCGATAG-3′

The 8 plasmids constructed as described above were each transformed into the strain E. coli BAP1 pTac-Pg6MSAS pWAS-anti-pabA showing the highest production, and then flask culture of the strain was performed. The results of the flask culture are shown in FIG. 15A. When C. glutamicum acetyl-CoA carboxylase was overexpressed, the production of 6-methylsalicylic acid increased to 63.6 mg/L. Fed-batch fermentation of the corresponding strain was performed, and as a result, as shown in FIG. 15B, the production of 6-methylsalicylic acid from glycerol increased to 440.3 mg/L±30.2 mg/L.

3.2. Increased Production of Aloesone Using Selected Knockdown Gene Targets

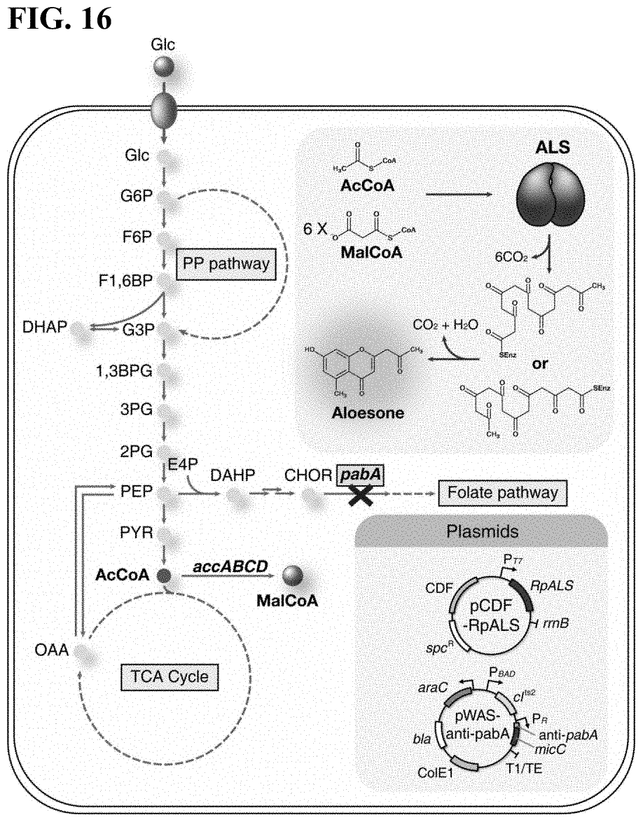

As a second product, aloesone produced from Rheum palmatum (Abe I, Utsumi Y, Oguro S, Noguchi H (2004), FEBS Lett 562:171-176) or Aloe arborescens (Mizuuchi Y, et al. (2009), FEBS J 276:2391-2401) type III polyketide synthase was tested. Aloesone is a precursor of aloesin which is widely used in the cosmetic field due its whitening effect. However, a biosynthetic pathway for producing aloesin from aloesone has not yet been reported. It has been reported that aloesone is produced from one molecule of acetyl-CoA and six molecules of malonyl-CoA (FIG. 16) and is produced by R. palmatum aloesone synthase (ALS) or A. arborescens aloesone synthase (PKS3).

The sequence of R. palmatum aloesone synthase (ALS) is as follows.

R. palmatum aloesone synthase (ALS)[SEQ ID NO:
125]:
madvlqeirn sqkasgpatv laigtahppt cypqadypdf
yfrvcksehm tklkkkmqfi cdrsgirqrf mfhteenlgk
npgmctfdgp slnarqdmli mevpklgaea aekaikewgq
dksrithlif ctttsndmpg adyqfatlfg lnpgvsrtmv
yqqgcfaggt vlrlvkdiae nnkgarvlvv cseivafafr
gphedhidsl igqllfgdga aalvvgtdid esverpifqi
msatqatipn slhtmalhlt eagltfhlsk evpkvvsdnm
eelmleafkp lgitdwnsif wqvhpggrai ldkieeklel
tkdkmrdsry ilseygnlts acvlfvmdem rkrsfregkq
ttgdgyewgv aiglgpgltv etvvlrsvpi p

In addition, the sequence of A. arborescens aloesone synthase (PKS3) is as follows.

A. arborescens aloesone synthase, PKS3) [SEQ ID NO:
126]:
mgslsdstpl mkdvqgirka qkadgtatvm aigtahpphi
isqdsyadfy frvtnsehkv elkkkfdric kktmigkryf
nfdeeflkky pnitsfdkps lndrhdicip gvpalgaeaa
vkaieewgrp kseithlvfc tsggvdmpsa dfqcakllgl
rtnvnkyciy mqgcyaggtv mryakdlaen nrgarvlmvc
aeltiialrg pndshidnai gnslfgdgaa alivgsdpii
gvekpmfeiv cakqtvipns eevihlhlre sglmfymtkd
saatisnnie aclvdvfksv gmtppedwns lfwiphpggr
aildqveakl klrpekfsat rtvlwdygnm isacvlyild

| | |
|---|---|
| emrrksaaeg | letygeglew gvllgfgpgm tietillhsl ppv |

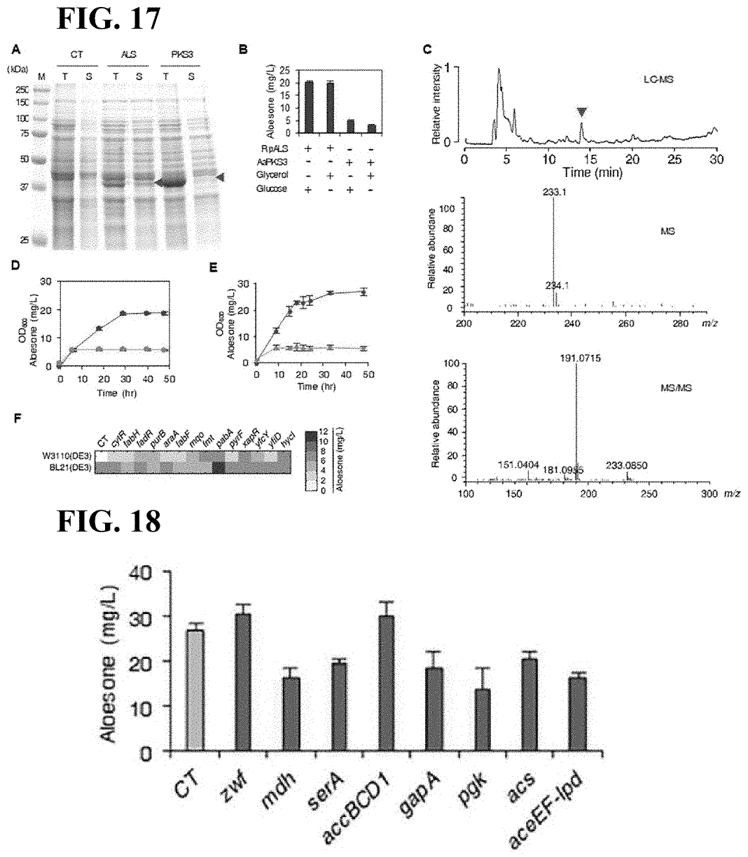

Genes corresponding to the two enzymes were each synthesized by Integrated DNA Technologies Inc. (USA) and then inserted by Gibson assembly into a pCDFDuet-1 (Novagen) plasmid at the NcoI site. The two constructed plasmids pCDF-RpALS and pCDF-AaPKS3 were each transformed into an E. coli BL21(DE3) strain, and then the expression of the heterologous enzymes was analyzed by SDS-PAGE (FIG. 17A). Flask culture of the strains was performed, and as a result, 20.5 mg/L and 4.7 mg/L of aloesone were produced from 20 g/L of glucose (FIG. 17B). Accordingly, the plasmid pCDF-RpALS was used in the following experiments. The authenticity of the produced aloesone was confirmed by LC-MS and MS/MS analysis (FIG. 17C). Next, the 14 sRNAs selected in the above Example were introduced into various E. coli strains as described in Example 3.1 above. To this end, each sRNA was introduced into a BL21(DE3) strain and a W3110(DE3) strain, thereby constructing 28 strains. The results of test tube-scale culture of the constructed strains are shown in FIG. 17F. At this time, the pabA-knockdown BL21(DE3) strain showed the highest aloesone production (18.5 mg/L), and the aloesone production of the strain in flask culture was 27.1 mg/L, which corresponds to an increase of 32.2% compared to that of the control strain (containing no sRNA) (FIGS. 17D and 17E).

In order to further increase the production of aloesone, the expression of the three FVSEOF gene targets (zwf, mdh, and serA; FIG. 10) and the following five enzymes used for increased production of 6-methylsalicylic acid was increased: C. glutamicum acetyl-CoA carboxylase (AccBC and AccD1), E. coli glyceraldehyde 3-phosphate dehydrogenase (GapA), E. coli phosphoglycerate kinase (Pgk), E. coli acetyl-CoA synthetase (Acs), and E. coli pyruvate dehydrogenase (PDH: AceEF and Lpd). To this end, eight plasmids pBBR1-zwf, pBBR1-mdh, pBBR1-serA, pBBR1-accBCD1, pBBR1-gapA, pBBR1-pgk, pBBR1-acs, and pBBR1-aceEF-lpd containing the corresponding genes were each transformed into the strain showing the highest aloesone production (E. coli BL21(DE3) harboring pCDF-RpALS and pWAS-anti-pabA), and then flask culture of the resulting strains was performed. The results of the flask culture are shown in FIG. 18. When C. glutamicum acetyl-CoA carboxylase was overexpressed, the production of aloesone increased to 30.9 mg/L. In this case, 20 g/L of glucose was used as a carbon source.

3.3. Increased Production of Resveratrol Using Selected Knockdown Gene Targets

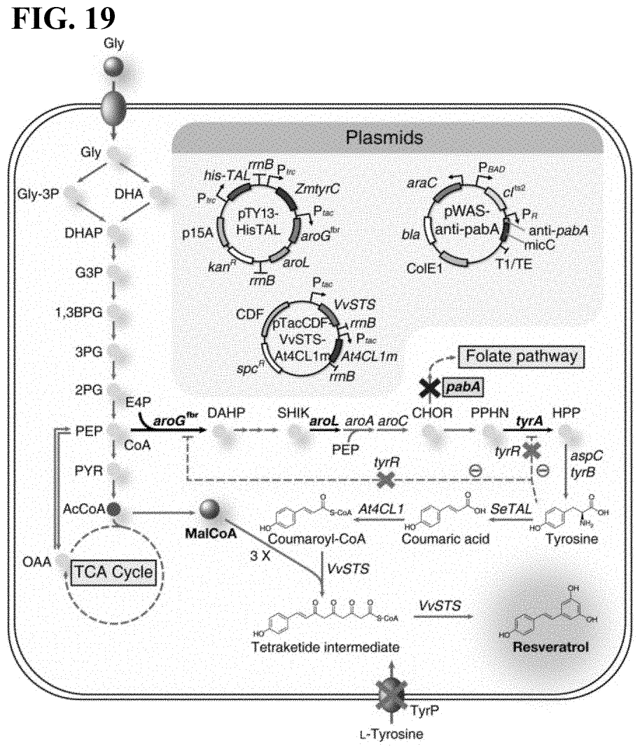

As a third product, resveratrol produced in plants was selected. Resveratrol, a compound belonging to the stilbenoid family among phenylpropanoid-based natural products, is a very useful natural product having antioxidant, anti-aging and anticancer effects, etc. Resveratrol is produced from one molecule of p-coumaroyl-CoA and three molecules of malonyl-CoA (FIG. 19). Accordingly, a strain that produces p-coumaric acid from a simple carbon source was constructed. To this end, a previously constructed tyrosine overproducing strain was used (Kim B, Binkley R, Kim H U, Lee S Y (2018), Biotechnol Bioeng). This strain (BTY5.13) contains a pTac15K plasmid-based plasmid (pTY13) which overexpresses Zymomonas mobilis tyrC, E. coli aroG^{fbr}, and aroL in a tyrR, tyrP-knockdown strain (BTY5) based on E. coli BL21(DE3). To convert tyrosine to p-coumaric acid, tyrosine ammonia-lyase (TAL) should be expressed. Thus, the gene SeTAL encoding tyrosine ammonia-lyase was amplified from the genomic DNA of Saccharothrix espanaensis using the primers of SEQ ID NO: 36 and SEQ ID NO: 37, and then a pTrc99A plasmid was linearized by PCR using the primers of SEQ ID NO: 38 and SEQ ID NO: 39. Next, the two DNA fragments were assembled using Gibson assembly, thereby constructing the plasmid pTrc-SeTAL.

The sequence of tyrosine ammonia-lyase that was used in the present invention is as follows.

Tyrosine ammonia-lyase (TAL) [SEQ ID NO: 127]:
mtqvverqad rlssreylar vvrsagwdag ltsctdeeiv
rmgasartie eylksdkpiy gltqgfgplv lfdadseleq
ggslishlgt gqgaplapev srlilwlriq nmrkgysays
pvfwqkladl wnkgftpaip rhgtvsasgd lqplahaala
ftgvgeawtr dadgrwstvp avdalaalga epfdwpvrea
lafvngtgas lavavlnhrs alrlvracav lsarlatllg
anpehydvgh gvargqvgql taaewirqgl prgmvrdgsr
plqepyslrc apqvlgavld qldgagdvla revdgcqdnp
ityegellhg gnfhampvgf asdqiglamh maaylaerql
gllvspvtng dlppmltpra grgaglagvq isatsfvsri
rqlvfpaslt tlptngwnqd hvpmalngan svfealelgw
ltvgslavgv aqlaamtgha aegvwaelag icppldadrp
lgaevraard llsahadqll vdeadgkdfg

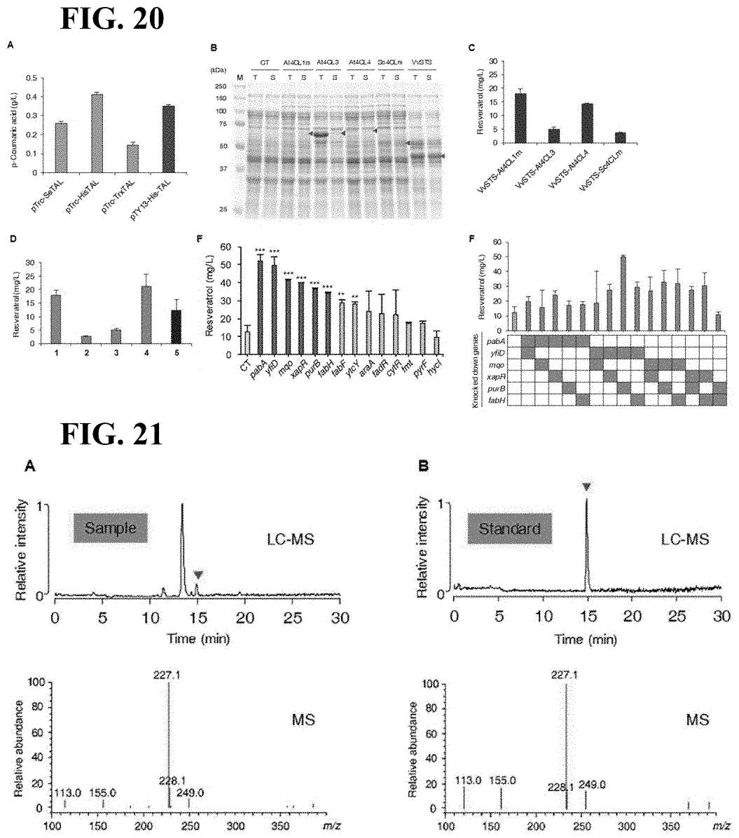

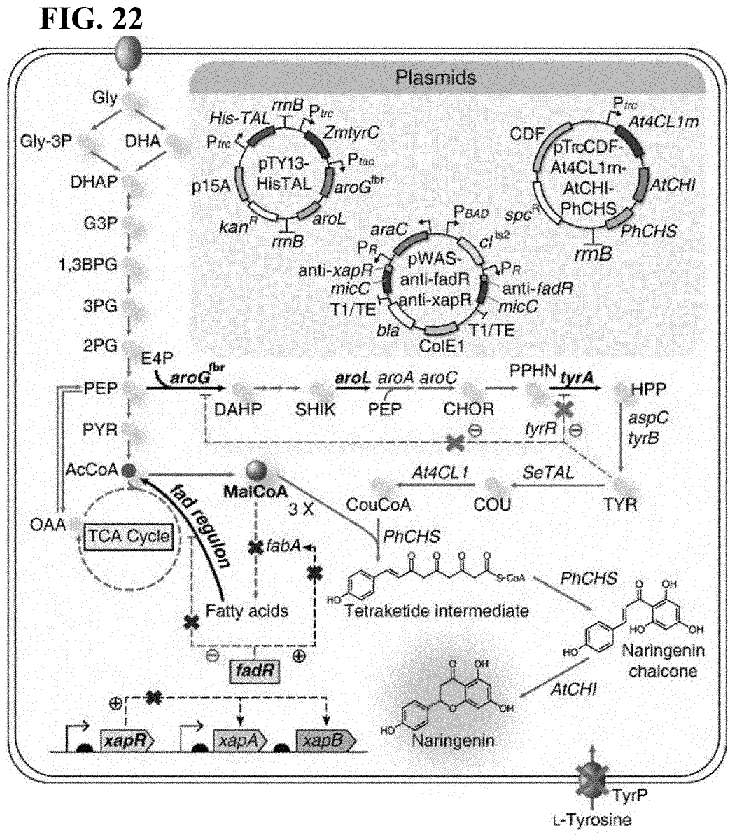

In order to optimize the expression of tyrosine ammonia-lyase, His-tag and thioredoxin tag (TrxA) were each attached to the N-terminus. For attachment of His-tag, His-SeTAL was amplified by PCR using the genomic DNA of S. espanaensis and the primers of SEQ ID NO: 40 and SEQ ID NO: 37, and pTrc-SeTAL as a template was linearized by inverse PCR using the primers of SEQ ID NO: 38 and SEQ ID NO: 41. The two DNA fragments were assembled using Gibson assembly, thereby constructing pTrc-HisTAL. Next, trxA gene was amplified by PCR using the genomic DNA of E. coli W3110 as a template and the primers of SEQ ID NO: 42 and SEQ ID NO: 43, and TrxA-TAL was amplified by PCR using the genomic DNA of S. espanaensis as a template and the primers of SEQ ID NO: 44 and SEQ ID NO: 45. The two DNA fragments were subjected to extension PCR to obtain a single DNA fragment by PCR using the primers of SEQ ID NO: 46 and SEQ ID NO: 37. The obtained DNA fragment was assembled by Gibson assembly with the linearized pTrc-SeTAL DNA fragment used for construction of the pTrc-HisTAL, thereby constructing pTrc-TrxTAL. The three constructed plasmids pTrc-SeTAL, pTrc-HisTAL and pTrc-TrxTAL were each transformed into a BTY5.13 strain, and then inoculated into a flask containing 50 mL of modified MR medium supplemented with 20 g/L of glucose, and the strains were cultured at 30° C. and 200 rpm. When the cells reached an OD₆₀₀ value of about 0.8, the cells were treated with 1 mM of IPTG, and then cultured for 36 hours. The modified MR medium contained, per liter, the following components: 6.67 g KH₂PO₄, 4 g (NH₄)₂HPO₄, 0.8 g citric acid, 0.8 g MgSO₄.7H₂O, 5 mL trace metal solution, 2 g yeast extract and 15 g (NH₄)₂SO₄. When His-TAL was expressed, the highest level of p-coumaric acid production (0.41 g/L) could be obtained (FIG. 20A). Thus, to construct a single plasmid by assembling pTrc-HisTAL with the pTY13 plasmid, the HisTAL portion was amplified using the primers of SEQ ID NO: 47 and SEQ ID NO: 48, and then inserted by Gibson assembly into the pTY13 plasmid at the NheI site, thereby constructing a pTY13-HisTAL plasmid. The constructed plasmid was transformed into a BTY5 strain, and then flask culture of the strain was performed. As a result, 0.35 g/L of p-coumaric acid was produced from 20 g/L of glycerol (FIG. 20A).

[SEQ ID NO: 36]
5′-GAATTGTGAGCGGATAACAAAGACCGAGGAAAAGGAGCATCGCAAATG
ACGCAGGTCGTGGAACGTC-3′
[SEQ ID NO: 37]
5′-TAGAGGATCCCCGGGTACTCATCCGAAATCCTTCCCGTC-3′
[SEQ ID NO: 38]
5′-GTACCCGGGGATCCTCTAG-3′
[SEQ ID NO: 39]
5′-TTGTTATCCGCTCACAATTC-3′
[SEQ ID NO: 40]
5′-GAGGAAAAGGAGCATCGCAAATGCACCATCATCATCATCAT
ACGCAGGTCGTGGAACGTC-3′
[SEQ ID NO: 41]
5′-TTGCGATGCTCCTTTTCCTC-3′
[SEQ ID NO: 42]
5′-ATGAGCGATAAAATTATTCACCTG-3′
[SEQ ID NO: 43]
5′-CGCCAGGTTAGCGTCGAGG-3′
[SEQ ID NO: 44]
5′-CCTCGACGCTAACCTGGCGACGCAGGTCGTGGAACGTC-3′
[SEQ ID NO: 45]
5′-TCATCCGAAATCCTTCCCGTC-3′
[SEQ ID NO: 46]
5′-AGACCGAGGAAAAGGAGCATCGCAAATGAGCGATAAAATTATTCACCT
G-3′
[SEQ ID NO: 47]
5′-GTAAGCCAGTATACACTCCGGACTGCACGGTGCACCAATG-3′
[SEQ ID NO: 48]
5′-CTGTTGGGCGCCATCTCCTTGTGTAGAAACGCAAAAAGGCCATC-3′

After successfully constructing the p-coumaric acid producing strain as described above, a downstream resveratrol biosynthetic pathway starting from p-coumaric acid was constructed, which is composed of Arabidopsis thaliana mutant 4-coumarate:CoA ligase (4CL) 1 (At4CL1m) and Vitis vinifera stilbene synthase (STS). First, At4CL1 gene was amplified from A. thaliana cDNA using the primers of SEQ ID NO: 49 and SEQ ID NO: 50, and then inserted by Gibson assembly into a pTac15K plasmid at EcoRI and KpnI sites, thereby constructing a pTac-At4CL1 plasmid. Next, pTac-At4CL1m (At4CL1m; I250L/N404K/I461V) was constructed by three consecutive rounds of site-directed mutagenesis in order to enhance substrate specificity for p-coumaroyl-CoA. At this time, for the three consecutive rounds of site-directed mutagenesis, the primer pairs of SEQ ID NO: 51 and SEQ ID NO: 52, SEQ ID NO: 53 and SEQ ID NO: 54, and SEQ ID NO: 55 and SEQ ID NO: 56 were used. Next, from A. thaliana, At4CL3 and At4CL4 genes that encode 4-coumarate:CoA ligase 3 and 4-coumarate:CoA ligase 4, respectively, were amplified by PCR using the primer pairs of SEQ ID NO: 57 and SEQ ID NO: 58, and SEQ ID NO: 59 and SEQ ID NO: 60, respectively, and from S. coelicolor, the Sc4CL gene encoding 4-coumarate:CoA ligase was amplified by PCR using the primer pair of SEQ ID NO: 61 and SEQ ID NO: 62. The amplified genes were cloned in the same manner as described above, thereby constructing pTac-At4CL3, pTac-At4CL4, and pTac-Sc4CL. For pTac-Sc4CL, one round of site-directed mutagenesis was performed using the primer pair of SEQ ID NO: 63 and SEQ ID NO: 64 in order to enhance substrate specificity for p-coumaroyl-CoA (Kaneko M, Ohnishi Y, Horinouchi S (2003), J Bacteriol 185:20-27), thereby constructing a pTac-Sc4CLm(Sc4CLm; A294G/A318G) plasmid. The STS gene encoding Vitis vinifera stilbene synthase was synthesized by Integrated DNA Technologies Inc., and it was amplified by PCR using the primer pair of SEQ ID NO: 65 and SEQ ID NO: 66, and then inserted into a pTac15K plasmid at EcoRI and KpnI sites using Gibson assembly, thereby constructing a pTac-VvSTS plasmid.

The sequence of A. thaliana 4-coumarate:CoA ligase 1 that was used in the present invention is as follows.

A. thaliana 4-coumarate: CoA ligase 1 [SEQ ID NO:
128]:
mapqeqavsq vmekqsnnnn sdvifrsklp diyipnhlsl
hdyifqnise fatkpcling ptghvytysd vhvisrqiaa
nfhklgvnqn dvvmlllpnc pefvlsflaa sfrgatataa
npfftpaeia kqakasntkl iitearyvdk ikplqnddgv
vivciddnes vpipegclrf teltqsttea sevidsveis
pddvvalpys sgttglpkgv mlthkglvts vaqqvdgenp
nlyfhsddvi lcvlpmfhiy alnsimlcgl rvgaailimp
kfeinlllel iqrckvtvap mvppivlaia kssetekydl
ssirvvksga aplgkeleda vnakfpnakl gqgygmteag
pvlamslgfa kepfpvksga cgtvvrnaem kivdpdtgds
lsrnqpgeic irghqimkgy lnnpaataet idkdgwlhtg
diglidddde lfivdrlkel ikykgfqvap aelealligh
pditdvavva mkeeaagevp vafvvkskds elseddvkqf
vskqvvfykr inkvfftesi pkapsgkilr kdlraklang l

The sequence of A. thaliana 4-coumarate:CoA ligase 3 that was used in the present invention is as follows.

A. thaliana 4-coumarate: CoA ligase 3 [SEQ ID NO:
129]:
mitaalhepq ihkptdtsvv sddvlphspp tprifrsklp
didipnhlpl htycfeklss vsdkpclivg stgksytyge
thlicrrvas glyklgirkg dvimillqns aefvfsfmga
smigaystta npfytsqely kqlkssgakl iithsqyvdk
lknlgenltl ittdeptpen clpfstlitd detnpfgetv
diggddaaal pfssgttglp kgvvlthksl itsvaqqvdg
dnpnlylksn dvilcvlplf hiyslnsvll nslrsgatvl
lmhkfeigal ldliqrhrvt iaalvpplvi alaknptvns
ydlssvrfvl sgaaplgkel qdslrrrlpq ailgqgygmt
eagpvlsmsl gfakepiptk sgscgtvvrn aelkvvhlet
rlslgynqpg eicirgqqim keylndpeat satideegwl
htgdigyvde ddeifivdrl kevikfkgfq vppaelesll
inhhsiadaa vvpqndevag evpvafvvrs ngnditeedv
keyvakqvvf ykrlhkvffv asipkspsgk ilrkdlkakl c

The sequence of A. thaliana 4-coumarate:CoA ligase 4 that was used in the present invention is as follows.

A. thaliana 4-coumarate: CoA ligase 4 [SEQ ID NO:
130]:
mvlqqqthfl tkkidqedee eepshdfifr sklpdifipn
hlpltdyvfq rfsgdgdgds sttciidgat griltyadvq
tnmrriaagi hrlgirhgdv vmlllpnspe falsflavay
lgaysttanp fytqpeiakq akasaakmii tkkclvdklt
nlkndgvliv cldddgdngv vsssddgcvs fteltqadet
ellkpkispe dtvampyssg ttglpkgvmi thkglvtsia
qkvdgenpnl nftandvilc flpmfhiyal dalmlsamrt
gaallivprf elnlvmeliq rykvtvvpva ppvvlafiks
peterydlss vrimlsgaat lkkeledavr lkfpnaifgq
gygmtesgtv akslafaknp fktksgacgt virnaemkvv
dtetgislpr nksgeicvrg hqlmkgylnd peatartidk
dgwlhtgdig fvddddeifi vdrlkelikf kgyqvapael
eallishpsi ddaavvamkd evadevpvaf varsqgsqlt
eddvksyvnk qvvhykrikm vffievipka vsgkilrkdl
rakletmcsk

The sequence of S. coelicolor 4-coumarate:CoA ligase that was used in the present invention is as follows.

S. coelicolor 4-coumarate: CoA ligase [SEQ ID NO:
131]:
mfrseyadvp pvdlpihdav lggaaafgst palidgtdgt
tltyeqvdrf hrrvaaalae tgvrkgdvla lhspntvafp
lafyaatrag asvttvhpla taeefakqlk dsaarwivtv
spllstarra aelaggvqei lvcdsapghr slvdmlasta
pepsvaidpa edvaalpyss gttgtpkgvm lthrgiatnl
aqlepsmpsa pgdrvlavlp ffhiygltal mnaplrlgat
vvvlprfdle qflaaiqnhr itslyvappi vlalakhplv
adydlsslry ivsaaaplda rlaaacsqrl glppvgqayg
mtelspgthv vpldamadap pgtvgrliag temrivsltd
pgtdlpages geilirgpqi mkgylgrpda taamideegw
lhtgdvghvd adgwlfvvdr vkelikykgf qvapaeleah
llthpgvada avvgaydddg nevphafvvr qpaapglaes
eimmyvaery apykrvrrvt fvdavpraas gkilrrqlre pr

The sequence of stilbene synthase that was used in the present invention is as follows.

Stilbene synthase [SEQ ID NO: 132]:
masveefrna qrakgpatil aigtatpdhc vyqsdyadfy
frvtksehmt alkkkfnric dksmikkryi hlteemleeh
pnigaymaps lnirqeiita evpklgkeaa lkalkewgqp
kskithlvfc ttsgvempga dyklanllgl epsvrrvmly
hqgcyaggtv lrtakdlaen nagarvlvvc seitvvtfrg
psedaldslv gqalfgdgsa avivgsdpdi sierplfqlv
saaqtfipns agaiagnlre vgltfhlwpn vptlisenie
kcltqafdpl gisdwnslfw iahpggpail daveaklnld
kkkleatrhv lseygnmssa cvlfildemr kkslkgerat
tgegldwgvl fgfgpgltie tvvlhsipmv tn

Each of the above-described plasmids was transformed into a BL21(DE3) strain, and then the expression of heterologous enzymes in the resulting strains was analyzed using SDS-PAGE. Then, in order to assemble the 4CL gene with the STS gene into a single plasmid, the following operation was performed. First, a DNA fragment (containing tac promoter) for stilbene synthase expression was amplified by PCR from pTac-VvSTS using the primers of SEQ ID NO: 47 and SEQ ID NO: 48, and then inserted into pTac-At4CL3, pTac-At4CL4l and pTac-Sc4CLm plasmid at an NheI site by Gibson assembly, thereby constructing pTac-VvSTS-At4CL3, pTac-VvSTS-At4CL4 and pTac-VvSTS-Sc4CLm plasmids. For At4CL1m, a DNA fragment for 4-coumarate:CoA ligase expression was amplified by PCR from pTac-At4CL1m using the primers of SEQ ID NO: 67 and SEQ ID NO: 68, and then inserted into a pTac-VvSTS plasmid at the PvuII site, thereby constructing a pTac-VvSTS-At4CL1m plasmid. The constructed plasmids pTac-VvSTS-At4CL1m, pTac-VvSTS-At4CL3, pTac-VvSTS-At4CL4, and pTac-VvSTS-Sc4CLm was transformed into a BL21(DE3) strain, and flask culture of the strain was performed in medium supplemented with 2 mM p-coumaric acid). The results of the flask culture are shown in FIG. 20C. At this time, the BL21(DE3) pTac-VvSTS-At4CL1m strain showed the highest resveratrol production (18.0 mg/L). The fact that resveratrol was produced in this amount even though p-coumaric acid was added at a concentration of 2 mM (328.1 mg/L) was added was an indirect evidence that malonyl-CoA was a bottleneck. In the present invention, before solving the problem according to this assumption by increasing the concentration of malonyl-CoA, this problem was solved by regulating the expression of exogenous genes for resveratrol production in various ways, and then malonyl-CoA was addressed. Thus, in order to express the At4CL1m gene and the STS gene in a single operon, the STS gene was amplified by PCR using the primers of SEQ ID NO: 69 and SEQ ID NO: 70, and then inserted into a pTac-At4CL1m plasmid at the SphI site, thereby constructing a pTac-At4CL1m-opr-VvSTS plasmid. In addition, in order to facilitate the transfer of a substrate by expressing the two enzymes as a single fused enzyme, the At4CL1m gene was amplified by PCR using the primer pair of SEQ ID NO: 71 and SEQ ID NO: 72, and then inserted into a pTac-VvSTS plasmid at the NdeI site, thereby constructing a pTac-At4CL1m-fus-VvSTS plasmid. For expression of the fusion protein, a glycine-serine linker (Gly-Gly-Gly-Ser) was used. However, neither of the two strategies produced a higher level of resveratrol (FIG. 20D). Thus, pTac-VvSTS-At4CL1m was selected as a final plasmid, and this plasmid was not compatible with pTY13-HisTAL (the two plasmid all have a p15A replication origin), and thus was transferred to the above-mentioned pTacCDFS plasmid. To this end, an STS expression cassette was amplified by PCR using the primers of SEQ ID NO: 73 and SEQ ID NO: 74, and then assembled by Gibson assembly with a pTacCDFS plasmid linearized using the primers of SEQ ID NO: 75 and SEQ ID NO: 76. The constructed plasmid was treated with PstI restriction enzyme, and then assembled by Gibson assembly or T4 ligation with an At4CL1m expression cassette amplified using the primers of SEQ ID NO: 75 and SEQ ID NO: 76, thereby constructing a single plasmid (pTacCDF-VvSTS-At4CL1m). The constructed plasmid was transformed into a BL21(DE3) strain, and then flask culture of the strain was performed, thereby producing 21.2 mg/L of resveratrol (FIG. 20D). However, this is the result of supplying 2 mM p-coumaric acid. Thus, in order to produce resveratrol directly from glycerol, the pTacCDF-VvSTS-At4CL1m plasmid was transformed into a BTY5 pTY13-HisTAL strain, and flask culture of the strain was performed, and as a result, 12.4 mg/L of resveratrol was produced from 20 g/L of glycerol. The authenticity of the produced resveratrol was confirmed by LC-MS (FIG. 21).

[SEQ ID NO: 49]
5′-CAATTTCACACAGGAAACAGACATATGGCGCCACAAGAACAAGC-3′
[SEQ ID NO: 50]
5′-CTCTAGAGGATCCCCGGGTACCATTACAATCCATTTGCTAGTTTTG-
3′
[SEQ ID NO: 51]
5′-CACAGCGATGACGTCCTACTCTGTGTTTTG-3′
[SEQ ID NO: 52]
5′-CAAAACACAGAGTAGGACGTCATCGCTGTG-3′
[SEQ ID NO: 53]
5′-CTCTTTCGAGGAAACAACCCGGTGAG-3′
[SEQ ID NO: 54]
5′-CTCACCGGGTTGTTTCCTCGAAAGAG-3′
[SEQ ID NO: 55]
5′-GATTGAAAGAACTTGTCAAGTATAAAGG-3′
[SEQ ID NO: 56]
5′-CCTTTATACTTGACAAGTTCTTTCAATC-3′
[SEQ ID NO: 57]
5′-CAATTTCACACAGGAAACAGACATATGATCACTGCAGCTCTACAC-3′
[SEQ ID NO: 58]
5′-CTCTAGAGGATCCCCGGGTACCATTAACAAAGCTTAGCTTTGAGG-3′
[SEQ ID NO: 59]
5′-CAATTTCACACAGGAAACAGACATATGGTGCTCCAACAACAAACG-3′
[SEQ ID NO: 60]
5′-CTCTAGAGGATCCCCGGGTACCATTATTTAGAGCACATGGTTTCC-3′
[SEQ ID NO: 61]
5′-CAATTTCACACAGGAAACAGACATATGTTCCGCAGCGAGTACGC-3′
[SEQ ID NO: 62]
5′-CTCTAGAGGATCCCCGGGTACCATTATCGCGGCTCCCTGAGCTGTC-
3′
[SEQ ID NO: 63]
5′-GTACATCGTCAGCGGCGCCGCCCCGCTCGACG-3′
[SEQ ID NO: 64]
5′-CGTCGAGCGGGGCGGCGCCGCTGACGATGTAC-3′
[SEQ ID NO: 65]
5′-CAATTTCACACAGGAAACAGACATATGGCAAGTGTCGAGGAATTC-3′
[SEQ ID NO: 66]
5′-CTCTAGAGGATCCCCGGGTACCATTAATTGGTAACCATCGGAATGG-
3′
[SEQ ID NO: 67]
5′-GTAAGCCAGTATACACTCCGGACTGCACGGTGCACCAATG-3′
[SEQ ID NO: 68]
5′-CTGTTGGGCGCCATCTCCTTGTGTAGAAACGCAAAAAGGCCATC-3′
[SEQ ID NO: 69]
5′-GAGTCGACCTGCAGGCATGCAATTTCACACAGGAAACAGA-3′
[SEQ ID NO: 70]
5′-CAAAACAGCCAAGCTTGCATG-3′
[SEQ ID NO: 71]
5′-TTTCACACAGGAAACAGACATATG-3′
[SEQ ID NO: 72]
5′-GAATTCCTCGACACTTGCCATACTACCACCACCCAATCCATTTGCTAG
TTTTG-3′
[SEQ ID NO: 73]
5′-GTTGACAATTAATCATCGGC-3′
[SEQ ID NO: 74]
5′-CGTTTCACTTCTGAGTTCGG-3′
[SEQ ID NO: 75]
5′-CCGAACTCAGAAGTGAAACG-3′
[SEQ ID NO: 76]
5′-GCCGATGATTAATTGTCAAC-3′

The 14 sRNAs selected in Example 2.1 above were introduced into the final resveratrol producing strain constructed as described above, and flask culture of the strain was performed. The results of the flask culture are shown in FIG. 20E. As a result, when pabA was knocked down, 51.8 mg/L of resveratrol was produced from glycerol, and this production was 4.2-fold increase compared to that in the control strain (a strain having no sRNA). In addition, it is notable that pabA is a knockdown target which showed the greatest increase in the production of both 6-methylsalicylic acid and aloesone as mentioned above. Next, six knockdown gene targets enabling more than 2.5-fold increase in resveratrol titer were selected, and sRNA plasmids, each containing a combination of two among the gene targets, were constructed. Each of the sRNA plasmids was transformed into a resveratrol producing strain, and then flask culture of each of the resulting strains was performed. The results of the flask culture are shown in FIG. 20. However, an additional increase in resveratrol production was not achieved, because as the highest resveratrol production was 50.0 mg/L when yfiD and purB were simultaneously knocked down.

Here, the sRNA plasmids for combinatorial double knockdown were constructed as follows. A DNA fragment encoding a synthetic-control sRNA to be inserted was amplified from a parent plasmid using the primers of SEQ ID NO: 77 and SEQ ID NO: 78, and then assembled by Gibson assembly with a synthetic-control sRNA-containing parent plasmid linearized by PCR using the primers of SEQ ID NO: 79 and SEQ ID NO: 80.

[SEQ ID NO: 77]
5′-GAATTTTAACAAAATATTAACGAATTCTAACACCGTGCGTG-3′
[SEQ ID NO: 78]
5′-GTGCCACCTAAATTGTAAGCGGCGAATTGGGTACCTATAAAC-3′
[SEQ ID NO: 79]
5′-GCTTACAATTTAGGTGGCAC-3′
[SEQ ID NO: 80]
5′-GTTAATATTTTGTTAAAATTCGCG-3′

3.4. Increased Production of Naringenin Using Selected Knockdown Gene Targets

As a fourth product, naringenin produced in plants was selected. Naringenin is a common precursor of many pharmacologically useful products belonging to the flavonoid family among phenylpropanoid-based natural products. In addition, naringenin was reported to have anti-Alzheimer, anticancer, antioxidant and antifungal activities. Naringenin is produced from one molecule of p-coumaroyl-CoA and three molecules of malonyl-CoA, like resveratrol (FIG. 21). Since the p-coumaric acid producing strain was constructed in Example 3.3 above, a downstream naringenin biosynthetic pathway was constructed in the present example. Here, conversion from p-coumaric acid to p-coumaroyl-CoA was performed using A. thaliana mutant 4-coumarate:CoA ligase 1 (At4CL1m), and conversion from p-coumaroyl-CoA and malonyl-CoA to naringenin chalcone was performed using Petunia x hybria chalcone synthase (CHS), and conversion from naringenin chalcone to naringenin was performed using A. thaliana chalcone isomerase (CHI).

The sequence of chalcone synthase (CHS) that was used in the present invention is as follows.

Chalcone synthase (CHS) [SEQ ID NO: 133]:
mvtveeyrka qraegpatvm aigtatptnc vdqstypdyy
fritnsehkt dlkekfkrmc eksmikkrym hlteeilken
psmceymaps ldarqdivvv evpklgkeaa qkaikewgqp
kskithlvfc ttsgvdmpgc dyqltkllgl rpsvkrlmmy
qqgcfaggtv lrlakdlaen nkgarvlvvc seitavtfrg
pndthldslv gqalfgdgag aiiigsdpip gverplfelv
saaqtllpds hgaidghlre vgltfhllkd vpglisknie
ksleeafrpl sisdwnslfw iahpggpail dqveiklglk
peklkatrnv lsnygnmssa cvlfildemr kasakeglgt
tgeglewgvl fgfgpgltve tvvlhsvat

The sequence of chalcone isomerase (CHI) that was used in the present invention is as follows.

Chalcone isomerase (CHI) [SEQ ID NO: 134]:
msssnacasp spfpavtklh vdsvtfvpsv kspassnplf
lggagvrgld iqgkfviftv igvylegnav pslsvkwkgk
tteeltesip ffreivtgaf ekfikvtmkl pltgqqysek
vtencvaiwk qlglytdcea kavekfleif keetfppgss
ilfalsptgs ltvafskdds ipetgiavie nkllaeavle
siigkngvsp gtrlsvaerl sqlmmknkde kevsdhsvee
klaken

The At4CL1m gene encoding mutant 4-coumarate:CoA ligase 1 was amplified by PCR using A. thaliana cDNA as a template and the primers of SEQ ID NO: 81 and SEQ ID NO: 82, and the AtCHI gene encoding chalcone synthase was amplified by PCR using A. thaliana cDNA as a template and the primers of SEQ ID NO: 83 and SEQ ID NO: 84. Then, the two DNA fragments were assembled into a single DNA fragment by extension PCR using the primers of SEQ ID NO: 81 and SEQ ID NO: 84, and then inserted into a pTrc99A plasmid at KpnI and BamHI sites. In addition, the PhCHS gene encoding chalcone synthase was amplified by PCR using a DNA fragment (synthesized by Integrated DNA Technologies Inc.) as a template and the primers of SEQ ID NO: 85 and SEQ ID NO: 86, and then inserted into the BamHI and XbaI sites of the above-constructed plasmid, thereby constructing a pTrc-At4CL1m-AtCHI-PsCHS plasmid. For the same reason as described in Example 3.3 above, the plasmid pTrc-At4CL1m-AtCHI-PsCHS was digested with NcoI and PstI restriction enzymes so that it would be compatible with the plasmid in the p-coumaric acid producing strain. The produced DNA fragment was assembled by Gibson assembly with a pTrcCDFS plasmid linearized by inverse PCR using the primers of SEQ ID NO: 87 and SEQ ID NO: 88, thereby constructing a pTrcCDF-At4CL1m-AtCHI-PhCHS plasmid.

[SEQ ID NO: 81]
5′-AGACAGGGTACCATGGCGCCACAAGAACAAG-3′
[SEQ ID NO: 82]
5′-ATGTATATCTCCTTCTTAAAGTTAATTACAATCCATTTGCTAGTTTTG
CCC-3′
[SEQ ID NO: 83]
5′-TTAACTTTAAGAAGGAGATATACATATGTCTTCATCCAACGCCTGC-
3′
[SEQ ID NO: 84]
5′-AGACAGGGATCCTCAGTTCTCTTTGGCTAGTTTTTCC-3′
[SEQ ID NO: 85]
5′-AGAGAGGATCCATAACAATTCCCCATCTTAG-3′
[SEQ ID NO: 86]
5′-AGAGATCTAGATTAGGTAGCCACACTATGCAGAACC-3′
[SEQ ID NO: 87]
5′-GAAACTGCTTGTTCTTGTGGCGCCATGGTCTGTTTCCTGTGTG-3′
[SEQ ID NO: 88]
5′-CTAATCTAGAGTCGACCTGCAGGCATGCAAGCTTG-3′

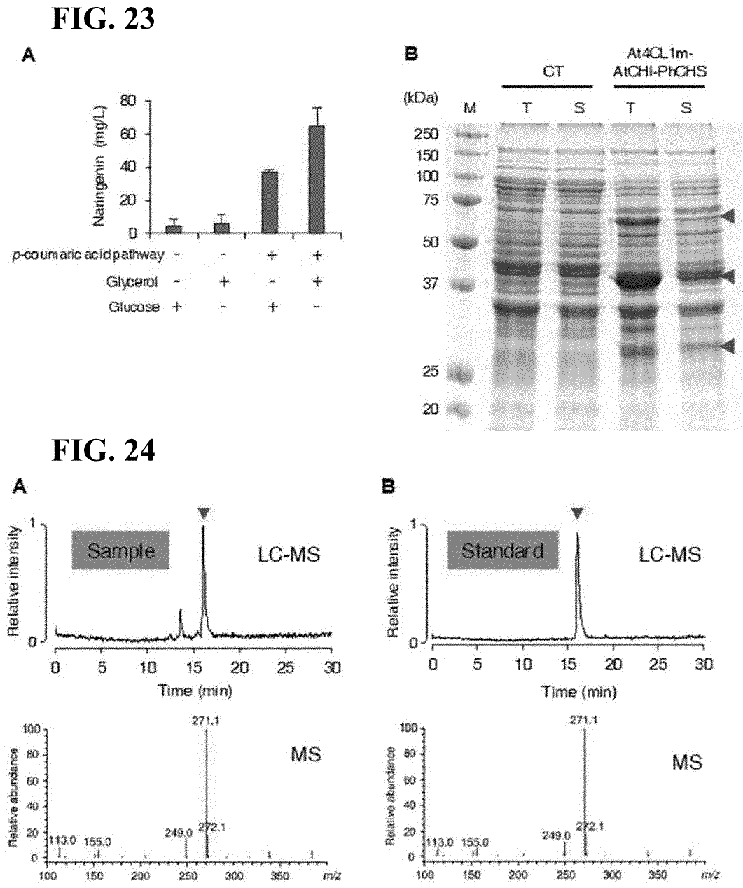

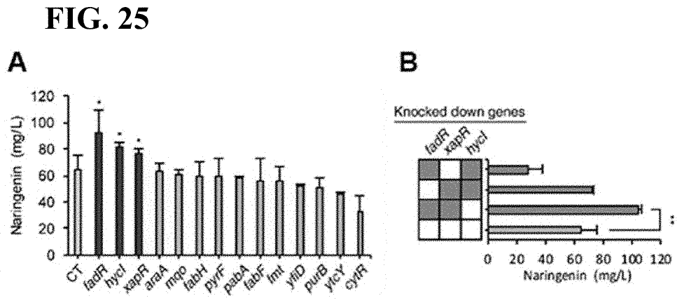

The plasmid pTrcCDF-At4CL1m-AtCHI-PhCHS constructed as described above was introduced into a BTY5 pTY13-HisTAL strain, and then flask culture of the strain was performed. As a result, 37.2 mg/L and 64.5 mg/L of naringenin were produced from glucose and glycerol, respectively (FIG. 23A). Thus, the following experiments on naringenin production were all performed using glycerol. The expression of heterologous enzymes in the strain was analyzed by SDS-PAGE (FIG. 23B), and the authenticity of the produced naringenin was confirmed by LC-MS (FIG. 24). The 14 sRNAs selected in Example 2.1 above were introduced into the constructed naringenin producing strain, and flask culture of each of the resulting strains was performed. The results of the flask culture are shown in FIG. 25A. At this time, the fadR-knockdown strain showed the highest naringenin production (92.3 mg/L), which was 43% increase in naringenin production compared to that in the control strain (a naringenin producing strain having no sRNA). In addition, knockdown targets showing more than 15% increase compared to the control were combined and subjected to a combinatorial double knockdown experiment, and the results of the experiment are shown in FIG. 25B. At this time, in the strain in which both fadR and xapR were knocked down, 103.8 mg/L of naringenin was produced from glycerol, which was 61% increase in naringenin production compared to that in the control.

Here, the sRNA plasmid for combinatorial double knockdown was constructed as follows. A DNA fragment encoding a synthetic-control sRNA to be inserted was amplified from the parent plasmid using the primers of SEQ ID NO: 89 and SEQ ID NO: 90, and then assembled by Gibson assembly with a synthetic-control sRNA-containing parent plasmid linearized by PCR using the primers of SEQ ID NO: 91 and SEQ ID NO: 92.

[SEQ ID NO: 89]
5′-CACTAGATCTCAAATGTGCTGGAATTCTAACACCGTGCGTG-3′
[SEQ ID NO: 90]
5′-CCTTATAAATCAAACATGTGCGGCGAATTGGGTACCTATAAAC-3′
[SEQ ID NO: 91]
5′-GCACATGTTTGATTTATAAGGG-3′
[SEQ ID NO: 92]
5′-CAGCACATTTGAGATCTAGTGG-3′

As examined in Example 3 above, the knockdown targets for increased malonyl-CoA production, easily and rapidly screened by the RppA malonyl-CoA biosensor, all contributed greatly to the malonyl-CoA-based production of useful compounds. The four useful product-producing strains examined in the above Examples are not those constructed through many metabolic engineering strategies as reported in previous studies, but are those constructed in a short time by constructing basic production pathways and simply transforming sRNAs. The fact that the strains constructed in a sample manner as described above show remarkable producing ability is noteworthy in the art. In addition, it is obvious to those skilled in the art that the utility and applicability of the RppA biosensor are not limited only to the above-described examples.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

Conventional methods for measuring malonyl-CoA concentration have various disadvantages and limitations in that they are time-consuming and labor-intensive, require skilled technology, have low data reliability, have difficulty in large-volume experiments, and require high-performance devices. However, the use of the biosensor according to the present invention provides single-step signal generation, utilization in various microorganisms, utilization in self-fluorescent microorganisms, a simple construction method, and a simple screening method. In addition, when the present invention is combined with high-throughput screening, it has advantages in that strains having increased malonyl-CoA producing ability can be screened very easily and rapidly (˜3 days) and can be applied directly to the malonyl-CoA-based production of useful compounds.

1. A recombinant microorganism for indicating intracellular malonyl-CoA level in which a type III polyketide synthase-encoding gene is inserted in the genome of the microorganism or in which a recombinant vector containing the type III polyketide synthase-encoding gene is introduced.

2. The recombinant microorganism of claim 1, wherein the type III polyketide synthase is:
RppA derived from a microorganism selected from the group consisting of Streptomyces griseus, Streptomyces coelicolor, Streptomyces avermitilis, Saccharopolyspora erythraea, Streptomyces peucetius, and Streptomyces aculeolatus;
PhlD (polyketide synthase) derived from Pseudomonas fluorescens;
DpgA (polyketide synthase) derived from Amycolatopsis mediterranei;
ALS (aloesone synthase) derived from Rheum palmatum; or
PCS (5,7-dihydroxy-2-methylchromone synthase), OKS (octaketide synthase), PKS3 (aloesone synthase), PKS4 (octaketide synthase 2) or PKS5 (octaketide synthase 3), which is derived from Aloe arborescens.

3. The recombinant microorganism of claim 1, wherein a gene encoding the type III polyketide synthase is operably linked to a promoter selected from the group consisting of tac, trc, T7, BAD, λPR an Anderson synthetic promoter.

4. The recombinant microorganism of claim 1, wherein the recombinant microorganism is selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhodobacter, Zymomonas, Bacillus, Staphylococcus, Lactococcus, Streptococcus, Lactobacillus, Clostridium, Corynebacterium, Streptomyces, Bifidobacterium, Cyanobacterium, and Cyclobacterium.

5. A method for screening a malonyl-CoA production-inducing substance, comprising the steps of:
(a) culturing the recombinant microorganism of claim 1;
(b) adding a candidate substance to the recombinant microorganism;
(c) comparing the color of a culture supernatant of the recombinant microorganism after addition of the candidate substance with the color of a culture supernatant of the recombinant microorganism without addition of the candidate substance; and
(d) selecting the candidate substance as the malonyl-CoA production-inducing substance, when the culture supernatant of the recombinant microorganism after addition of the candidate substance shows a deeper red color than the culture supernatant of the recombinant microorganism without addition of the candidate substance.

6. The method of claim 5, wherein the comparison between the colors in step (c) is made by the naked eyes.

7. The method of claim 5, wherein the comparison between the colors in step (c) is made by measuring absorbance, and step (d) comprises selecting the candidate substance as the malonyl-CoA production-inducing substance, when the absorbance measured after addition of the candidate substance is higher than that measured without addition of the candidate substance.

8. A method for screening a gene involved in increase of malonyl-CoA production, the method comprising the steps of:
(a) introducing a gene regulation library, which changes gene expression in a recombinant microorganism, into the recombinant microorganism of claim 1, thereby constructing a recombinant microorganism library in which gene expression in the recombinant microorganism is changed;
(b) culturing the constructed recombinant microorganism library and the recombinant microorganism before introduction of the gene regulation library, and comparing the color of the culture supernatant of the recombinant microorganism library with the color of the culture supernatant of the recombinant microorganism before introduction of the gene regulation library; and
(c) selecting the gene introduced in the recombinant microorganism library as a gene involved in increase of malonyl-CoA production, when the culture supernatant of the recombinant microorganism library shows a deeper red color than the culture supernatant of the recombinant microorganism before introduction of the gene regulation library.

9. The method of claim 8, wherein the comparison between the colors in step (b) is made by the naked eyes.

10. The method of claim 8, wherein the comparison between the colors in step (b) is made by measuring absorbance, and step (c) comprises selecting the gene introduced in the recombinant microorganism library, when the absorbance of the culture supernatant of the recombinant microorganism library is higher than that of the culture supernatant of the recombinant microorganism before introduction of the gene regulation library.

11. The method of claim 8, wherein the gene regulation library is a library selected from an sRNA library, a genomic library, a cDNA library, a gRNA library, and an oligonucleotide library for construction of knockout or mutant strains.

12.-33. (canceled)


SEQUENCE LISTING (181 sequences)

SEQ ID NO: 1 - DNA, 41 bp, Artificial Sequence
  Primer
ctttaagaaggagatatacatatggcgaccctgtgccgacc

SEQ ID NO: 2 - DNA, 43 bp, Artificial Sequence
  Primer
cttgtcgacggagctcgaattcattagccggacagcgcaacgc

SEQ ID NO: 3 - DNA, 40 bp, Artificial Sequence
  Primer
cgactcctgcattaggaaatgactgcacggtgcaccaatg

SEQ ID NO: 4 - DNA, 20 bp, Artificial Sequence
  Primer
gcgtttcacttctgagttcg

SEQ ID NO: 5 - DNA, 41 bp, Artificial Sequence
  Primer
cgaactcagaagtgaaacgcctgaaacctcaggcatttgag

SEQ ID NO: 6 - DNA, 20 bp, Artificial Sequence
  Primer
atttcctaatgcaggagtcg

SEQ ID NO: 7 - DNA, 41 bp, Artificial Sequence
  Primer
caatttcacacaggaaacagaatggcgaccctgtgccgacc

SEQ ID NO: 8 - DNA, 43 bp, Artificial Sequence
  Primer
ctctagaggatccccgggtaccattagccggacagcgcaacgc

SEQ ID NO: 9 - DNA, 22 bp, Artificial Sequence
  Primer
ggtacccggggatcctctagag

SEQ ID NO: 10 - DNA, 21 bp, Artificial Sequence
  Primer
tctgtttcctgtgtgaaattg

SEQ ID NO: 11 - DNA, 44 bp, Artificial Sequence
  Primer
gatgctcctttcttgttattgaaattgttatccgctcacaattc

SEQ ID NO: 12 - DNA, 40 bp, Artificial Sequence
  Primer
cgaactcagaagtgaaacgctgcctaatgagtgagctaac

SEQ ID NO: 13 - DNA, 41 bp, Artificial Sequence
  Primer
cattggtgcaccgtgcagtcaaaattcgcgttaaatttttg

SEQ ID NO: 14 - DNA, 20 bp, Artificial Sequence
  Primer
gactgcacggtgcaccaatg

SEQ ID NO: 15 - DNA, 20 bp, Artificial Sequence
  Primer
gcgtttcacttctgagttcg

SEQ ID NO: 16 - DNA, 59 bp, Artificial Sequence
  Primer
caatttcacacaggaaacagaatgcaccatcaccatcaccatgcgaccctgtgccgacc

SEQ ID NO: 17 - DNA, 20 bp, Artificial Sequence
  Primer
ccattataattaggcctcgg

SEQ ID NO: 18 - DNA, 20 bp, Artificial Sequence
  Primer
ccatgctactcctaccaacc

SEQ ID NO: 19 - DNA, 45 bp, Artificial Sequence
  Primer
ggttggtaggagtagcatgggatccatggcgaccctgtgccgacc

SEQ ID NO: 20 - DNA, 41 bp, Artificial Sequence
  Primer
ccgaggcctaattataatggattagccggacagcgcaacgc

SEQ ID NO: 21 - DNA, 48 bp, Artificial Sequence
  Primer
ggttggtaggagtagcatgggatccatgcaccatcaccatcaccatgc

SEQ ID NO: 22 - DNA, 47 bp, Artificial Sequence
  Primer
cttgatcagcttgcatgcctgcagaagctttctagcagaaataattc

SEQ ID NO: 23 - DNA, 27 bp, Artificial Sequence
  Primer
gtgcatgtggactccctttctcttatc

SEQ ID NO: 24 - DNA, 41 bp, Artificial Sequence
  Primer
gataagagaaagggagtccacatggcgaccctgtgccgacc

SEQ ID NO: 25 - DNA, 45 bp, Artificial Sequence
  Primer
ggatcctctagagtcgacctgcagattagccggacagcgcaacgc

SEQ ID NO: 26 - DNA, 44 bp, Artificial Sequence
  Primer
gataagagaaagggagtccacatgcaccatcaccatcaccatgc

SEQ ID NO: 27 - DNA, 43 bp, Artificial Sequence
  Primer
gctgagaagcttgccaaataatggatcctctagagtcgacctg

SEQ ID NO: 28 - DNA, 74 bp, Artificial Sequence
  Primer
ctggggatgttttcccagaggggtatgtagaagttgcagcggaatgcatgaattctgtttcctgtgtgaaattg

SEQ ID NO: 29 - DNA, 24 bp, Artificial Sequence
  Primer
acagtgaatatgaattctccaacg

SEQ ID NO: 30 - DNA, 22 bp, Artificial Sequence
  Primer
attatttggcaagcttctcagc

SEQ ID NO: 31 - DNA, 71 bp, Artificial Sequence
  Primer
ctctgggaaaacatccccagcaccagtcggaacccctgggactgagtacagtgaatatgaattctccaacg

SEQ ID NO: 32 - DNA, 36 bp, Artificial Sequence
  Primer
taataagaattcatgaagatttacggaatttatatg

SEQ ID NO: 33 - DNA, 34 bp, Artificial Sequence
  Primer
ttattagaattcttataaaagctcttcgtacgag

SEQ ID NO: 34 - DNA, 43 bp, Artificial Sequence
  Primer
ctagagtcgacctgcaggcatgccactcccgttctggataatg

SEQ ID NO: 35 - DNA, 22 bp, Artificial Sequence
  Primer
caaaacagccaagcttgcatgc

SEQ ID NO: 36 - DNA, 67 bp, Artificial Sequence
  Primer
gaattgtgagcggataacaaagaccgaggaaaaggagcatcgcaaatgacgcaggtcgtggaacgtc

SEQ ID NO: 37 - DNA, 39 bp, Artificial Sequence
  Primer
tagaggatccccgggtactcatccgaaatccttcccgtc

SEQ ID NO: 38 - DNA, 19 bp, Artificial Sequence
  Primer
gtacccggggatcctctag

SEQ ID NO: 39 - DNA, 20 bp, Artificial Sequence
  Primer
ttgttatccgctcacaattc

SEQ ID NO: 40 - DNA, 60 bp, Artificial Sequence
  Primer
gaggaaaaggagcatcgcaaatgcaccatcatcatcatcatacgcaggtcgtggaacgtc

SEQ ID NO: 41 - DNA, 20 bp, Artificial Sequence
  Primer
ttgcgatgctccttttcctc

SEQ ID NO: 42 - DNA, 24 bp, Artificial Sequence
  Primer
atgagcgataaaattattcacctg

SEQ ID NO: 43 - DNA, 19 bp, Artificial Sequence
  Primer
cgccaggttagcgtcgagg

SEQ ID NO: 44 - DNA, 38 bp, Artificial Sequence
  Primer
cctcgacgctaacctggcgacgcaggtcgtggaacgtc

SEQ ID NO: 45 - DNA, 21 bp, Artificial Sequence
  Primer
tcatccgaaatccttcccgtc

SEQ ID NO: 46 - DNA, 49 bp, Artificial Sequence
  Primer
agaccgaggaaaaggagcatcgcaaatgagcgataaaattattcacctg

SEQ ID NO: 47 - DNA, 40 bp, Artificial Sequence
  Primer
gtaagccagtatacactccggactgcacggtgcaccaatg

SEQ ID NO: 48 - DNA, 44 bp, Artificial Sequence
  Primer
ctgttgggcgccatctccttgtgtagaaacgcaaaaaggccatc

SEQ ID NO: 49 - DNA, 44 bp, Artificial Sequence
  Primer
caatttcacacaggaaacagacatatggcgccacaagaacaagc

SEQ ID NO: 50 - DNA, 46 bp, Artificial Sequence
  Primer
ctctagaggatccccgggtaccattacaatccatttgctagttttg

SEQ ID NO: 51 - DNA, 30 bp, Artificial Sequence
  Primer
cacagcgatgacgtcctactctgtgttttg

SEQ ID NO: 52 - DNA, 30 bp, Artificial Sequence
  Primer
caaaacacagagtaggacgtcatcgctgtg

SEQ ID NO: 53 - DNA, 26 bp, Artificial Sequence
  Primer
ctctttcgaggaaacaacccggtgag

SEQ ID NO: 54 - DNA, 26 bp, Artificial Sequence
  Primer
ctcaccgggttgtttcctcgaaagag

SEQ ID NO: 55 - DNA, 28 bp, Artificial Sequence
  Primer
gattgaaagaacttgtcaagtataaagg

SEQ ID NO: 56 - DNA, 28 bp, Artificial Sequence
  Primer
cctttatacttgacaagttctttcaatc

SEQ ID NO: 57 - DNA, 45 bp, Artificial Sequence
  Primer
caatttcacacaggaaacagacatatgatcactgcagctctacac

SEQ ID NO: 58 - DNA, 45 bp, Artificial Sequence
  Primer
ctctagaggatccccgggtaccattaacaaagcttagctttgagg

SEQ ID NO: 59 - DNA, 45 bp, Artificial Sequence
  Primer
caatttcacacaggaaacagacatatggtgctccaacaacaaacg

SEQ ID NO: 60 - DNA, 45 bp, Artificial Sequence
  Primer
ctctagaggatccccgggtaccattatttagagcacatggtttcc

SEQ ID NO: 61 - DNA, 44 bp, Artificial Sequence
  Primer
caatttcacacaggaaacagacatatgttccgcagcgagtacgc

SEQ ID NO: 62 - DNA, 46 bp, Artificial Sequence
  Primer
ctctagaggatccccgggtaccattatcgcggctccctgagctgtc

SEQ ID NO: 63 - DNA, 32 bp, Artificial Sequence
  Primer
gtacatcgtcagcggcgccgccccgctcgacg

SEQ ID NO: 64 - DNA, 32 bp, Artificial Sequence
  Primer
cgtcgagcggggcggcgccgctgacgatgtac

SEQ ID NO: 65 - DNA, 45 bp, Artificial Sequence
  Primer
caatttcacacaggaaacagacatatggcaagtgtcgaggaattc

SEQ ID NO: 66 - DNA, 46 bp, Artificial Sequence
  Primer
ctctagaggatccccgggtaccattaattggtaaccatcggaatgg

SEQ ID NO: 67 - DNA, 40 bp, Artificial Sequence; identical to SEQ ID NO: 47
  Primer

SEQ ID NO: 68 - DNA, 44 bp, Artificial Sequence; identical to SEQ ID NO: 48
  Primer

SEQ ID NO: 69 - DNA, 40 bp, Artificial Sequence
  Primer
gagtcgacctgcaggcatgcaatttcacacaggaaacaga

SEQ ID NO: 70 - DNA, 21 bp, Artificial Sequence
  Primer
caaaacagccaagcttgcatg

SEQ ID NO: 71 - DNA, 24 bp, Artificial Sequence
  Primer
tttcacacaggaaacagacatatg

SEQ ID NO: 72 - DNA, 53 bp, Artificial Sequence
  Primer
gaattcctcgacacttgccatactaccaccacccaatccatttgctagttttg

SEQ ID NO: 73 - DNA, 20 bp, Artificial Sequence
  Primer
gttgacaattaatcatcggc

SEQ ID NO: 74 - DNA, 20 bp, Artificial Sequence
  Primer
cgtttcacttctgagttcgg

SEQ ID NO: 75 - DNA, 20 bp, Artificial Sequence
  Primer
ccgaactcagaagtgaaacg

SEQ ID NO: 76 - DNA, 20 bp, Artificial Sequence
  Primer
gccgatgattaattgtcaac

SEQ ID NO: 77 - DNA, 41 bp, Artificial Sequence
  Primer
gaattttaacaaaatattaacgaattctaacaccgtgcgtg

SEQ ID NO: 78 - DNA, 42 bp, Artificial Sequence
  Primer
gtgccacctaaattgtaagcggcgaattgggtacctataaac

SEQ ID NO: 79 - DNA, 20 bp, Artificial Sequence
  Primer
gcttacaatttaggtggcac

SEQ ID NO: 80 - DNA, 24 bp, Artificial Sequence
  Primer
gttaatattttgttaaaattcgcg

SEQ ID NO: 81 - DNA, 31 bp, Artificial Sequence
  Primer
agacagggtaccatggcgccacaagaacaag

SEQ ID NO: 82 - DNA, 51 bp, Artificial Sequence
  Primer
atgtatatctccttcttaaagttaattacaatccatttgctagttttgccc

SEQ ID NO: 83 - DNA, 46 bp, Artificial Sequence
  Primer
ttaactttaagaaggagatatacatatgtcttcatccaacgcctgc

SEQ ID NO: 84 - DNA, 37 bp, Artificial Sequence
  Primer
agacagggatcctcagttctctttggctagtttttcc

SEQ ID NO: 85 - DNA, 31 bp, Artificial Sequence
  Primer
agagaggatccataacaattccccatcttag

SEQ ID NO: 86 - DNA, 36 bp, Artificial Sequence
  Primer
agagatctagattaggtagccacactatgcagaacc

SEQ ID NO: 87 - DNA, 43 bp, Artificial Sequence
  Primer
gaaactgcttgttcttgtggcgccatggtctgtttcctgtgtg

SEQ ID NO: 88 - DNA, 35 bp, Artificial Sequence
  Primer
ctaatctagagtcgacctgcaggcatgcaagcttg

SEQ ID NO: 89 - DNA, 41 bp, Artificial Sequence
  Primer
cactagatctcaaatgtgctggaattctaacaccgtgcgtg

SEQ ID NO: 90 - DNA, 43 bp, Artificial Sequence
  Primer
ccttataaatcaaacatgtgcggcgaattgggtacctataaac

SEQ ID NO: 91 - DNA, 22 bp, Artificial Sequence
  Primer
gcacatgtttgatttataaggg

SEQ ID NO: 92 - DNA, 22 bp, Artificial Sequence
  Primer
cagcacatttgagatctagtgg

SEQ ID NO: 93 - protein, 372 aa, Streptomyces griseus
MATLCRPAIAVPEHVITMQQTLDLARETHAGHPQRDLVLRLIQNTGVQTRHLVQPIEKTLAHPGFEVRNQVYEAEAKTRVPEVVRRALANAETEPSEIDLIVYVSCTGFMMPSLTAWIINSMGFRPETRQLPIAQLGCAAGGAAINRAHDFCVAYPDSNVLIVSCEFCSLCYQPTDIGVGSLLSNGLFGDALSAAVVRGQGGTGMRLERNGSHLVPDTEDWISYAVRDTGFHFQLDKRVPGTMEMLAPVLLDLVDLHGWSVPNMDFFIVHAGGPRILDDLCHFLDLPPEMFRYSRATLTERGNIASSVVFDALARLFDDGGAAESAQGLIAGFGPGITAEVAVGSWAKEGLGADVGRDLDELELTAGVALSG

SEQ ID NO: 94 - protein, 374 aa, Streptomyces coelicolor
MATLCRPSVSVPEHVITMEETLELARRRHTDHPQLPLALRLIENTGVRTRHIVQPIEDTLEHPGFEDRNKVYEREAKSRVPAVIQRALDDAELLATDIDVIIYVSCTGFMMPSLTAWLINEMGFDSTTRQIPIAQLGCAAGGAAINRAHDFCTAYPEANALIVACEFCSLCYQPTDLGVGSLLCNGLFGDGIAAAVVRGRGGTGVRLERNGSYLIPKTEDWIMYDVKATGFHFLLDKRVPATMEPLAPALKELAGEHGWDASDLDFYIVHAGGPRILDDLSTFLEVDPHAFRFSRATLTEYGNIASAVVLDALRRLFDEGGVEEGARGLLAGFGPGITAEMSLGCWQTADVRRGIRQDVTRTAARGVSRRVRQA

SEQ ID NO: 95 - protein, 352 aa, Streptomyces avermitilis
MATLCKPAVSVPEHVITMEETLELARSRHPDHPQLPLALRLIENTGVHTRHIVQPIEETLKHPGFEERNHVYEAEAKARVPAVVQRALDEAELLTTDIDVIIYVSCTGFMMPSLTAYLINSMDFSSDTRQIPIAQLGCAAGGSAINRAHDFCTAYPQANALIVACEFCSLCYQPTDLGVGSLLSNGLFGDGIAAAAVRGKGGTGITLERNASYLIPKTDEWISYDVRATGFHFLLDKRVPGTMEPLAPALQELASQHGWDASDLDFYIIHAGGPRILDDLSKFLRVPPEAFRFSRATLTEYGNIASAVVLDALRRLFDEGGAEHAARGMLAGFGPGITAEMSLGRWHRTDEA

SEQ ID NO: 96 - protein, 367 aa, Saccharopolyspora erythraea
MAVLCTPAVAVPEHVITVEETLDLARRVHADHPQLPLVLRLISNTGVRERHLIRPIEDTLEHPGFEVRNRIYEEQAKQRVPAVVREALDSAELGPEDIDLIVYVSCTGFMMPSLTAWLINSMGFRMSTRQLPIAQLGCAAGGAAINRAHDFCTAYPDANALIVSCEFCSLCYQPTDDDIGSLLSNGLFGDAVGAAVVRGHGGTGVRLERNASSMIPETEDWISYAVKATGFHFQLDKRVPKTMEPLAPALRALAEDHRWDVAGLDFYVIHAGGPRILDDLTKFLGVPSEAFRHSRATLAQYGNIASAVVLDALRRIIEEGRLESGARGMIAGFGPGITAEMSVGTWVPHDVLLHGEHSTTSAPGGNR

SEQ ID NO: 97 - protein, 377 aa, Streptomyces peucetius
MRVPVAVDDLVAPSTMGERHTVIDRGTSVAAVHTALPPHRYAQSDLTELIADLCLEPGADRALLRRLHTSAGVRTRHLALPIEQYAGLGDFGQANAAWLTVGLALAEEALSGALDAAGLTAADIDLLVCTSITGVAAPSLDARLAVRMGMRADVKRVPVFGLGCVGGAAGLGRLHDYLLGHPDDTAVLLSVELCSLTLQRDGSLANLVAGALFGDGAAAVVARGGDAGRRGAGWPMVAATRGHLYPDTEHLLGWRIGASGFRVVVDAGIPDVVRTHLGGDLRNFLATHGLVPDDIGTWICHPGGPKVLAAVGDALELPDGALDSSWRSLAGVGNLSSASVLRVLEDVATRCRPDPGTWGVLLAMGPGFCAEFVLLRW

SEQ ID NO: 98 - protein, 355 aa, Streptomyces aculeolatus
MPRLCKPAVSAPEYTITMEETLEFAKQAHAGKPQLPLALRLIRNTGVLKRHIVQPIEKTLGHPGLTERNLIYEAESKKMCPPVIEEALQNADMTARDIDAIIYVSCTGFLMPSLTAWLINKMGFRSDTRQIPIAQLGCAAGGAAVNRAHDFCLAHPGSNVLIVACELCSLCYQPTADDIGSLLSDGLFGDAVAAAVVRGNGGVGIEVERNASYLIPNTEEWISYSVRDTGFHFQLDRRVPGTMEPLAPVLREFAKDHSWDAGKLDFYIVHAGGPRILDDLARFLDVDRQVFRHSWSTLTEYGNIASAVVFDAARRLFEEGSAKPDATGMIAGFGPGITAEMALGTWGTDGTGTSN

SEQ ID NO: 99 - protein, 349 aa, Pseudomonas fluorescens
MSTLCKPSLLFPHYKITQQQMIDHLEQLHDDHPRMALAKRMIQNTQVNERYLVLPIDELAVHTGFTHRSIVYEREARRMSSIAARQAIENAGLTTDDIRMVAVTSCTGFMMPSLTAHLINDLGLRTSTVQLPIAQLGCVAGAAAINRANDFASLSPDNHALIVSLEFSSLCYQPQDTKLHAFISAALFGDAVSACVMRADDKAPGFKIAKTGSYFLPDSEHYIKYDVKDSGFHFTLDKAVMNSIKDVAPMMEELNFETFNQHCAQNDFFIFHTGGRKILDELVLQLDLEPGRVAQSRDSLSEAGNIASVVVFDVLKRQFDSGPANGATGMLAAFGPGFTAEMAVGKWVA

SEQ ID NO: 100 - protein, 370 aa, Amycolatopsis orientalis
MDVSMTTGIELTEELSVLNGLTEITRFAGVGTAVSETSYSQTELLDILDVEDPKIRSVFLNSAIDRRFLTLPPENPGGGRLAEPQGDLLDKHKKIAVDMGCRALEACLKSAGATLSDLRHLCCVTSTGFLTPGLSALIIREMGIDPHCSRSDIVGMGCNAGLNALNVVSGWSAAHPGELGVVLCSEACSAAYALDGTMRTAVVNSLFGDGSAALAVISGDGRVAGPRVLKFASYIITDAVDAMRYDWDRDQDRFSFFLDPQIPYVVGAHAEIVVDRLLSGTGLRRSDIGHWLVHSGGKKVVDAVVVNLGLSRHDVRHTTGVLRDYGNLSSGSFLFSYERLSEEDVTRPGDYGVLMTMGPGSTIEMALIQW

SEQ ID NO: 101 - protein, 391 aa, Unknown
  Rheum palmatum
MADVLQEIRNSQKASGPATVLAIGTAHPPTCYPQADYPDFYFRVCKSEHMTKLKKKMQFICDRSGIRQRFMFHTEENLGKNPGMCTFDGPSLNARQDMLIMEVPKLGAEAAEKAIKEWGQDKSRITHLIFCTTTSNDMPGADYQFATLFGLNPGVSRTMVYQQGCFAGGTVLRLVKDIAENNKGARVLVVCSEIVAFAFRGPHEDHIDSLIGQLLFGDGAAALVVGTDIDESVERPIFQIMSATQATIPNSLHTMALHLTEAGLTFHLSKEVPKVVSDNMEELMLEAFKPLGITDWNSIFWQVHPGGRAILDKIEEKLELTKDKMRDSRYILSEYGNLTSACVLFVMDEMRKRSFREGKQTTGDGYEWGVAIGLGPGLTVETVVLRSVPIP

SEQ ID NO: 102 - protein, 403 aa, Aloe arborescens
MSSLSNSLPLMEDVQGIRKAQKADGTATVMAIGTAHPPHIFPQDTYADVYFRATNSEHKVELKKKFDHICKKTMIGKRYFNYDEEFLKKYPNITSYDEPSLNDRQDICVPGVPALGTEAAVKAIEEWGRPKSEITHLVFCTSCGVDMPSADFQCAKLLGLHANVNKYCIYMQGCYAGGTVMRYAKDLAENNRGARVLVVCAELTIMMLRAPNETHLDNAIGISLFGDGAAALIIGSDPIIGVEKPMFEIVCTKQTVIPNTEDVIHLHLRETGMMFYLSKGSPMTISNNVEACLIDVFKSVGITPPEDWNSLFWIPHPGGRAILDQVEAKLKLRPEKFRAARTVLWDYGNMVSASVGYILDEMRRKSAAKGLETYGEGLEWGVLLGFGPGITVETILLHSLPLM

SEQ ID NO: 103 - protein, 403 aa, Aloe arborescens
MSSLSNASHLMEDVQGIRKAQRADGTATVMAIGTAHPPHIFPQDTYADFYFRATNSEHKVELKKKFDRICKKTMIGKRYFNYDEEFLKKYPNITSFDEPSLNDRQDICVPGVPALGAEAAVKAIAEWGRPKSEITHLVFCTSCGVDMPSADFQCAKLLGLRTNVNKYCVYMQGCYAGGTVMRYAKDLAENNRGARVLVVCAELTIIGLRGPNESHLDNAIGNSLFGDGAAALIVGSDPIIGVEKPMFEIVCAKQTVIPNSEDVIHLHMREAGLMFYMSKDSPETISNNVEACLVDVFKSVGMTPPEDWNSLFWIPHPGGRAILDQVEAKLKLRPEKFRATRTVLWDCGNMVSACVLYILDEMRRKSADEGLETYGEGLEWGVLLGFGPGMTVETILLHSLPLM

SEQ ID NO: 104 - protein, 403 aa, Aloe arborescens
MGSLSDSTPLMKDVQGIRKAQKADGTATVMAIGTAHPPHIISQDSYADFYFRVTNSEHKVELKKKFDRICKKTMIGKRYFNFDEEFLKKYPNITSFDKPSLNDRHDICIPGVPALGAEAAVKAIEEWGRPKSEITHLVFCTSGGVDMPSADFQCAKLLGLRTNVNKYCIYMQGCYAGGTVMRYAKDLAENNRGARVLMVCAELTIIALRGPNDSHIDNAIGNSLFGDGAAALIVGSDPIIGVEKPMFEIVCAKQTVIPNSEEVIHLHLRESGLMFYMTKDSAATISNNIEACLVDVFKSVGMTPPEDWNSLFWIPHPGGRAILDQVEAKLKLRPEKFSATRTVLWDYGNMISACVLYILDEMRRKSAAEGLETYGEGLEWGVLLGFGPGMTIETILLHSLPPV

SEQ ID NO: 105 - protein, 403 aa, Aloe arborescens
MGSLSNYSPVMEDVQAIRKAQKADGTATVMAIGTAHPPHIFPQDTYADFYFRATNSEHKVELKKKFDRICKKTMIGKRYFNYDEEFLKKYPNITSFDEPSLNDRQDICVPGVPALGAEAAVKAIAEWGRPKSEITHLVFCTSCGVDMPSADFQCAKLLGLRTNVNKYCVYMQGCYAGGTVMRYAKDLAENNRGARVLVVCAELTIIGLRGPNESHLDNAIGNSLFGDGAAALIVGSDPIIGVERPMFEIVCAKQTVIPNSEDVIHLHMREAGLMFYMSKDSPETISNNVEACLVDVFKSVGMTPPEDWNSLFWIPHPGGRAILDQVEARLKLRPEKFGATRTVLWDCGNMVSACVLYILDEMRRKSVADGLATYGEGLEWGVLLGFGPGMTVETILLHSLPPV

SEQ ID NO: 106 - protein, 405 aa, Aloe arborescens
MGSIAESSPLMSRENVEGIRKAQRAEGTATVMAIGTAHPPHIFPQDTYADFYFRATNSEHKVELKKKFDRICKKTMIGKRYFNYDEEFLKKYPNITSFDEPSLNDRQDICVPGVPALGKEAALKAIEEWGQPLSKITHLVFCTSCGVDMPSADFQLAKLLGLNTNVNKYCVYMQGCYAGGTVLRYAKDLAENNRGSRVLVVCAELTIIGLRGPNESHLDNAIGNSLFGDGAAALIVGADPIVGIEKPIFEIVCAKQTVIPDSEDVIHLHLREAGLMFYMSKDSPETISNNVEGCLVDIFKSVGMTPPADWNSLFWIPHPGGRAILDEVEARLKLRPEKFRATRHVLWEYGNMVSACVLYILDEMRNKSAADGLGTYGEGLEWGVLLGFGPGMTVETILLHSLPPV

SEQ ID NO: 107 - protein, 413 aa, Escherichia coli
MSKRRVVVTGLGMLSPVGNTVESTWKALLAGQSGISLIDHFDTSAYATKFAGLVKDFNCEDIISRKEQRKMDAFIQYGIVAGVQAMQDSGLEITEENATRIGAAIGSGIGGLGLIEENHTSLMNGGPRKISPFFVPSTIVNMVAGHLTIMYGLRGPSISIATACTSGVHNIGHAARIIAYGDADVMVAGGAEKASTPLGVGGFGAARALSTRNDNPQAASRPWDKERDGFVLGDGAGMLVLEEYEHAKKRGAKIYAELVGFGMSSDAYHMTSPPENGAGAALAMANALRDAGIEASQIGYVNAHGTSTPAGDKAEAQAVKTIFGEAASRVLVSSTKSMTGHLLGAAGAVESIYSILALRDQAVPPTINLDNPDEGCDLDFVPHEARQVSGMEYTLCNSFGFGGTNGSLIFKKI

SEQ ID NO: 108 - protein, 436 aa, Escherichia coli
MGQVLPLVTRQGDRIAIVSGLRTPFARQATAFHGIPAVDLGKMVVGELLARSEIPAEVIEQLVFGQVVQMPEAPNIAREIVLGTGMNVHTDAYSVSRACATSFQAVANVAESLMAGTIRAGIAGGADSSSVLPIGVSKKLARVLVDVNKARTMSQRLKLFSRLRLRDLMPVPPAVAEYSTGLRMGDTAEQMAKTYGITREQQDALAHRSHQRAAQAWSDGKLKEEVMTAFIPPYKQPLVEDNNIRGNSSLADYAKLRPAFDRKHGTVTAANSTPLTDGAAAVILMTESRAKELGLVPLGYLRSYAFTAIDVWQDMLLGPAWSTPLALERAGLTMSDLTLIDMHEAFAAQTLANIQLLGSERFAREALGRAHATGEVDDSKFNVLGGSIAYGHPFAATGARMITQTLHELRRRGGGFGLVTACAAGGLGAAMVLEAE

SEQ ID NO: 109 - protein, 294 aa, Escherichia coli
MERVYRTDLKLLRYFLAVAEELHFGRAAARLNMSQPPLSIHIKELENQLGTQLFIRHSRSVVLTHAGKILMEESRRLLVNANNVLARIEQIGRGEAGRIELGVVGTAMWGRMRPVMRRFLRENPNVDVLFREKMPAMQMALLERRELDAGIWRMATEPPTGFTSLRLHESAFLVAMPEEHHLSSFSTVPLEALRDEYFVTMPPVYTDWDFLQRVCQQVGFSPVVIREVNEPQTVLAMVSMGIGITLIADSYAQMNWPGVIFRPLKQRIPADLYIVYETQQVTPAMVKLLAALTQ

SEQ ID NO: 110 - protein, 341 aa, Escherichia coli [sequence omitted]

SEQ ID NO: 111 - protein, 317 aa, Escherichia coli
MYTKIIGTGSYLPEQVRTNADLEKMVDTSDEWIVTRTGIRERHIAAPNETVSTMGFEAATRAIEMAGIEKDQIGLIVVATTSATHAFPSAACQIQSMLGIKGCPAFDVAAACAGFTYALSVADQYVKSGAVKYALVVGSDVLARTCDPTDRGTIIIFGDGAGAAVLAASEEPGIISTHLHADGSYGELLTLPNADRVNPENSIHLTMAGNEVFKVAVTELAHIVDETLAANNLDRSQLDWLVPHQANLRIISATAKKLGMSMDNVVVTLDRHGNTSAASVPCALDEAVRDGRIKPGQLVLLEAFGGGFTWGSALVRF

SEQ ID NO: 112 - protein, 548 aa, Escherichia coli [sequence omitted]

SEQ ID NO: 113 - protein, 127 aa, Escherichia coli
MITGIQITKAANDDLLNSFWLLDSEKGEARCIVAKAGYAEDEVVAVSKLGDIEYREVPVEVKPEVRVEGGQHLNVNVLRRETLEDAVKHPEKYPQLTIRVSGYAVRFNSLTPEQQRDVIARTFTESL

SEQ ID NO: 114 - protein, 315 aa, Escherichia coli
MSESLRIIFAGTPDFAARHLDALLSSGHNVVGVFTQPDRPAGRGKKLMPSPVKVLAEEKGLPVFQPVSLRPQENQQLVAELQADVMVVVAYGLILPKAVLEMPRLGCINVHGSLLPRWRGAAPIQRSLWAGDAETGVTIMQMDVGLDTGDMLYKLSCPITAEDTSGTLYDKLAELGPQGLITTLKQLADGTAKPEVQDETLVTYAEKLSKEEARIDWSLSAAQLERCIRAFNPWPMSWLEIEGQPVKVWKASVIDTATNAAPGTILEANKQGIQVATGDGILNLLSLQPAGKKAMSAQDLLNSRREWFVPGNRLV

SEQ ID NO: 115 - protein, 245 aa, Escherichia coli
MTLTASSSSRAVTNSPVVVALDYHNRDDALAFVDKIDPRDCRLKVGKEMFTLFGPQFVRELQQRGFDIFLDLKFHDIPNTAAHAVAAAADLGVWMVNVHASGGARMMTAAREALVPFGKDAPLLIAVTVLTSMEASDLVDLGMTLSPADYAERLAALTQKCGLDGVVCSAQEAVRFKQVFGQEFKLVTPGIRPQGSEAGDQRRIMTPEQALSAGVDYMVIGRPVTQSVDPAQTLKAINASLQRSA

SEQ ID NO: 116 - protein, 500 aa, Escherichia coli
MTIFDNYEVWFVIGSQHLYGPETLRQVTQHAEHVVNALNTEAKLPCKLVLKPLGTTPDEITAICRDANYDDRCAGLVVWLHTFSPAKMWINGLTMLNKPLLQFHTQFNAALPWDSIDMDFMNLNQTAHGGREFGFIGARMRQQHAVVTGHWQDKQAHERIGSWMRQAVSKQDTRHLKVCRFGDNMREVAVTDGDKVAAQIKFGFSVNTWAVGDLVQVVNSISDGDVNALVDEYESCYTMTPATQIHGKKRQNVLEAARIELGMKRFLEQGGFHAFTTTFEDLHGLKQLPGLAVQRLMQQGYGFAGEGDWKTAALLRIMKVMSTGLQGGTSFMEDYTYHFEKGNDLVLGSHMLEVCPSIAAEEKPILDVQHLGIGGKDDPARLIFNTQTGPAIVASLIDLGDRYRLLVNCIDTVKTPHSLPKLPVANALWKAQPDLPTASEAWILAGGAHHTVFSHALNLNDMRQFAEMHDIEITVIDNDTRLPAFKDALRWNEVYYGFRR

SEQ ID NO: 117 - protein, 334 aa, Escherichia coli
MLNTLIVGASGYAGAELVTYVNRHPHMNITALTVSAQSNDAGKLISDLHPQLKGIVDLPLQPMSDISEFSPGVDVVFLATAHEVSHDLAPQFLEAGCVVFDLSGAFRVNDATFYEKYYGFTHQYPELLEQAAYGLAEWCGNKLKEANLIAVPGCYPTAAQLALKPLIDADLLDLNQWPVINATSGVSGAGRKAAISNSFCEVSLQPYGVFTHRHQPEIATHLGADVIFTPHLGNFPRGILETITCRLKSGVTQAQVAQVLQQAYAHKPLVRLYDKGVPALKNVVGLPFCDIGFAVQGEHLIIVATEDNLLKGAAAQAVQCANIRFGYAETQSLI

SEQ ID NO: 118 - protein, 239 aa, Escherichia coli
MVIKAQSPAGFAEEYIIESIWNNRFPPGTILPAERELSELIGVTRTTLREVLQRLARDGWLTIQHGKPTKVNNFWETSGLNILETLARLDHESVPQLIDNLLSVRTNISTIFIRTAFRQHPDKAQEVLATANEVADHADAFAELDYNIFRGLAFASGNPIYGLILNGMKGLYTRIGRHYFANPEARSLALGFYHKLSALCSEGAHDQVYETVRRYGHESGEIWHRMQKNLPGDLAIQGR

SEQ ID NO: 119 - protein, 159 aa, Escherichia coli
MFLRQEDFATVVRSTPLVSLDFIVENSRGEFLLGKRTNRPAQGYWFVPGGRVQKDETLEAAFERLTMAELGLRLPITAGQFYGVWQHFYDDNFSGTDFTTHYVVLGFRFRVSEEELLLPDEQHDDYRWLTSDALLASDNVHANSRAYFLAEKRTGVPGL

SEQ ID NO: 120 - protein, 187 aa, Escherichia coli
MILLIDNYDSFTWNLYQYFCELGADVLVKRNDALTLADIDALKPQKIVISPGPCTPDEAGISLDVIRHYAGRLPILGVCLGHQAMAQAFGGKVVRAAKVMHGKTSPITHNGEGVFRGLANPLTVTRYHSLVVEPDSLPACFDVTAWSETREIMGIRHRQWDLEGVQFHPESILSEQGHQLLANFLHR

SEQ ID NO: 121 - protein, 456 aa, Escherichia coli
MELSSLTAVSPVDGRYGDKVSALRGIFSEYGLLKFRVQVEVRWLQKLAAHAAIKEVPAFAADAIGYLDAIVASFSEEDAARIKTIERTTNHDVKAVEYFLKEKVAEIPELHAVSEFIHFACTSEDINNLSHALMLKTARDEVILPYWRQLIDGIKDLAVQYRDIPLLSRTHGQPATPSTIGKEMANVAYRMERQYRQLNQVEILGKINGAVGNYNAHIAAYPEVDWHQFSEEFVTSLGIQWNPYTTQIEPHDYIAELFDCVARFNTILIDFDRDVWGYIALNHFKQKTIAGEIGSSTMPHKVNPIDFENSEGNLGLSNAVLQHLASKLPVSRWQRDLTDSTVLRNLGVGIGYALIAYQSTLKGVSKLEVNRDHLLDELDHNWEVLAEPIQTVMRRYGIEKPYEKLKELTRGKRVDAEGMKQFIDGLALPEEEKARLKAMTPANYIGRAITMVDELK

SEQ ID NO: 122 - protein, 156 aa, Escherichia coli
MTDVLLCVGNSMMGDDGAGPLLAEKCAAAPKGNWVVIDGGSAPENDIVAIRELRPTRLLIVDATDMGLNPGEIRIIDPDDIAEMFMMTTHNMPLNYLIDQLKEDIGEVIFLGIQPDIVGFYYPMTQPIKDAVETVYQRLEGWEGNGGFAQLAVEEE

SEQ ID NO: 123 - protein, 1774 aa, Penicillium urticae
MHSAATSTYPSGKTSPAPVGTPGTEYSEYEFSNDVAVVGMACRVAGGNHNPELLWQSLLSQKSAMGEIPPMRWEPYYRRDARNEKFLKNTTSRGYFLDRLEDFDCQFFGISPKEAEQMDPQQRVSLEVASEALEDAGIPAKSLSGSDTAVFWGVNSDDYSKLVLEDLPNVEAWMGIGTAYCGVPNRISYHLNLMGPSTAVDAACASSLVAIHHGVQAIRLGESKVAIVGGVNALCGPGLTRVLDKAGAISSDGSCKSFDDDAHGYARGEGAGALVLKSLHRALLDHDNVLAVIKGSAVCQDGKTNGIMAPNSVAQQLAANNALSAANIDPHTVRYVEAHATSTPLGDPTEISAIASVYGADRPADDPCYIGSIKPNIGHLEAGAGVMGFIKAVLAIQKGVLPPQANLTKLNSRIDWKTAGVKVVQEATPWPESDPIRRAGVCSYGYGGTVSHAVIEEFSPILQPDPLGNGAVSGPGLLLLSGPQEKRLALQAKTLRDWMTAEGKDHNLSDILTTLATRRDHHDYRAALVVDDYRDAEQVLQSLANGVDHTFTTQSRVLGSDISKDVVWVFSGHGAQWPDMGKQLIHNPVFFAAIQPLDELIQAEIGLSPIELLRTGDFESSDRVQILTYVMQIGLSALLQSNGITPQAVIGHSVGEIAASVVAGALSPAEGALIVTRRALLYRQVMGKGGMILVNLPSAETEEILGSRSDLVVAIDSSPSSCVVAGDKELVAETAEALKARGVKTFTVKSDIAFHSPTLNGLVDPLRDVLAETLSPVSPNVKLYSTALADPRGQDLRDVEYWAGNMVNRVRLTSAVKAAVEDGYRLFLEVSTHPVVSHSINETLMDAGMEDFAVIPTLLRKKPTEKHILHSIAQLHCRGAEVNWAAQMPGRWATGVPTTTWMHKPIWRKIETAPLHTGLTHDVEKHTLLGQRIPVPGTDTYVYTTRLDNDTKPFPGSHPLHGTEIVPAAGLINTFLKGTGGQMLQNVVLRVPVAINAPRSVQVVVQQDQVKVVSRLIPSEPSQLDDDASWVTHTTAYWDRKVAGSEDRIDFAAVKSRLVTKLADNFSIDYLDKVGVSAMGFPWAVTEHYRNDKEMLARVDVNPAISGDAPLPWDSSSWAPVLDAATSVGSTIFPTPALRMPAQIERVEVFTSQDPPKISWLYVQEASDSVPTSHVSVVSEAGEVLAKFTAMRFSEIEGTPGVSGSMESLVHQIAWPPATPAEEPLSIETVILVSPDATTRALYAASLPTRVNSFQFSSTQEFFSNASSLPLEKGTVVTYIPGEVASLAEVPAASESFTWNLLELIKFTVNGSLPIKVFTLTANIGEGQTPTALAQSPLYGLARVIASEHPDLGTLIDVEEPVIPLSTMRYIQGADIIRINDGIARTSRFRSLPRNKLLPASEGPRLLPRPEGTYLITGGLGVLGLEVADFLVEKGARRLLLISRRALPPRRTWDQVSEDLQPTIAKIRLLESRGASVHVLPLDITKPDAVEQLTTALDRLSLPSVQGVVHAAGVLDNELVMQTTRDAFNRVLAPKIAGALALHEVFPPKSVDFFVMFSSCGNLVGFTGQASYGSGNAFLDTLATHRARLGDAAVSFQWTSWRGLGMGASTDFINAELESKGITDVTRDEAFAAWQHLAKYDMDHGVVLRSRAFEDGEPIPVSILNDIAVRRVGTVSNTSPAAAGSSDAVPTSGPELKAYLDEKIRGCVAKVLQMTAEDVDSKAALADLGVDSVMTVTLRRQLQLTLKIAVPPTLTWSHPTVSHLAVWFAEKLAK

SEQ ID NO: 124 - protein, 224 aa, Bacillus subtilis
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVICAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAVHPDFPEDITMVSYEELL

SEQ ID NO: 125 - protein, 391 aa, Unknown; identical to SEQ ID NO: 101
  Rheum palmatum

SEQ ID NO: 126 - protein, 403 aa, Aloe arborescens; identical to SEQ ID NO: 104

SEQ ID NO: 127 - protein, 510 aa, Unknown
  Saccharothrix espanaensis
MTQVVERQADRLSSREYLARVVRSAGWDAGLTSCTDEEIVRMGASARTIEEYLKSDKPIYGLTQGFGPLVLFDADSELEQGGSLISHLGTGQGAPLAPEVSRLILWLRIQNMRKGYSAVSPVFWQKLADLWNKGFTPAIPRHGTVSASGDLQPLAHAALAFTGVGEAWTRDADGRWSTVPAVDALAALGAEPFDWPVREALAFVNGTGASLAVAVLNHRSALRLVRACAVLSARLATLLGANPEHYDVGHGVARGQVGQLTAAEWIRQGLPRGMVRDGSRPLQEPYSLRCAPQVLGAVLDQLDGAGDVLAREVDGCQDNPITYEGELLHGGNFHAMPVGFASDQIGLAMHMAAYLAERQLGLLVSPVTNGDLPPMLTPRAGRGAGLAGVQISATSFVSRIRQLVFPASLTTLPTNGWNQDHVPMALNGANSVFEALELGWLTVGSLAVGVAQLAAMTGHAAEGVWAELAGICPPLDADRPLGAEVRAARDLLSAHADQLLVDEADGKDFG

SEQ ID NO: 128 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 129 - protein, 561 aa, Arabidopsis thaliana
MITAALHEPQIHKPTDTSVVSDDVLPHSPPTPRIFRSKLPDIDIPNHLPLHTYCFEKLSSVSDKPCLIVGSTGKSYTYGETHLICRRVASGLYKLGIRKGDVIMILLQNSAEFVFSFMGASMIGAVSTTANPFYTSQELYKQLKSSGAKLIITHSQYVDKLKNLGENLTLITTDEPTPENCLPFSTLITDDETNPFQETVDIGGDDAAALPFSSGTTGLPKGVVLTHKSLITSVAQQVDGDNPNLYLKSNDVILCVLPLFHIYSLNSVLLNSLRSGATVLLMHKFEIGALLDLIQRHRVTIAALVPPLVIALAKNPTVNSYDLSSVRFVLSGAAPLGKELQDSLRRRLPQAILGQGYGMTEAGPVLSMSLGFAKEPIPTKSGSCGTVVRNAELKVVHLETRLSLGYNQPGEICIRGQQIMKEYLNDPEATSATIDEEGWLHTGDIGYVDEDDEIFIVDRLKEVIKFKGFQVPPAELESLLINHHSIADAAVVPQNDEVAGEVPVAFVVRSNGNDITEEDVKEYVAKQVVFYKRLHKVFFVASIPKSPSGKILRKDLKAKLC

SEQ ID NO: 130 - protein, 570 aa, Arabidopsis thaliana
MVLQQQTHFLTKKIDQEDEEEEPSHDFIFRSKLPDIFIPNHLPLTDYVFQRFSGDGDGDSSTTCIIDGATGRILTYADVQTNMRRIAAGIHRLGIRHGDVVMLLLPNSPEFALSFLAVAYLGAVSTTANPFYTQPEIAKQAKASAAKMIITKKCLVDKLTNLKNDGVLIVCLDDDGDNGVVSSSDDGCVSFTELTQADETELLKPKISPEDTVAMPYSSGTTGLPKGVMITHKGLVTSIAQKVDGENPNLNFTANDVILCFLPMFHIYALDALMLSAMRTGAALLIVPRFELNLVMELIQRYKVTVVPVAPPVVLAFIKSPETERYDLSSVRIMLSGAATLKKELEDAVRLKFPNAIFGQGYGMTESGTVAKSLAFAKNPFKTKSGACGTVIRNAEMKVVDTETGISLPRNKSGEICVRGHQLMKGYLNDPEATARTIDKDGWLHTGDIGFVDDDDEIFIVDRLKELIKFKGYQVAPAELEALLISHPSIDDAAVVAMKDEVADEVPVAFVARSQGSQLTEDDVKSYVNKQVVHYKRIKMVFFIEVIPKAVSGKILRKDLRAKLETMCSK

SEQ ID NO: 131 - protein, 522 aa, Streptomyces coelicolor
MFRSEYADVPPVDLPIHDAVLGGAAAFGSTPALIDGTDGTTLTYEQVDRFHRRVAAALAETGVRKGDVLALHSPNTVAFPLAFYAATRAGASVTTVHPLATAEEFAKQLKDSAARWIVTVSPLLSTARRAAELAGGVQEILVCDSAPGHRSLVDMLASTAPEPSVAIDPAEDVAALPYSSGTTGTPKGVMLTHRQIATNLAQLEPSMPSAPGDRVLAVLPFFHIYGLTALMNAPLRLGATVVVLPRFDLEQFLAAIQNHRITSLYVAPPIVLALAKHPLVADYDLSSLRYIVSAAAPLDARLAAACSQRLGLPPVGQAYGMTELSPGTHVVPLDAMADAPPGTVGRLIAGTEMRIVSLTDPGTDLPAGESGEILIRGPQIMKGYLGRPDATAAMIDEEGWLHTGDVGHVDADGWLFVVDRVKELIKYKGFQVAPAELEAHLLTHPGVADAAVVGAYDDDGNEVPHAFVVRQPAAPGLAESEIMMYVAERVAPYKRVRRVTFVDAVPRAASGKILRRQLREPR

SEQ ID NO: 132 - protein, 392 aa, Vitis vinifera
MASVEEFRNAQRAKGPATILAIGTATPDHCVYQSDYADFYFRVTKSEHMTALKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLYHQGCYAGGTVLRTAKDLAENNAGARVLVVCSEITVVTFRGPSEDALDSLVGQALFGDGSAAVIVGSDPDISIERPLFQLVSAAQTFIPNSAGAIAGNLREVGLTFHLWPNVPTLISENIEKCLTQAFDPLGISDWNSLFWIAHPGGPAILDAVEAKLNLDKKKLEATRHVLSEYGNMSSACVLFILDEMRKKSLKGERATTGEGLDWGVLFGFGPGLTIETVVLHSIPMVTN

SEQ ID NO: 133 - protein, 389 aa, Petunia x hybrida
MVTVEEYRKAQRAEGPATVMAIGTATPTNCVDQSTYPDYYFRITNSEHKTDLKEKFKRMCEKSMIKKRYMHLTEEILKENPSMCEYMAPSLDARQDIVVVEVPKLGKEAAQKAIKEWGQPKSKITHLVFCTTSGVDMPGCDYQLTKLLGLRPSVKRLMMYQQGCFAGGTVLRLAKDLAENNKGARVLVVCSEITAVTFRGPNDTHLDSLVGQALFGDGAGAIIIGSDPIPGVERPLFELVSAAQTLLPDSHGAIDGHLREVGLTFHLLKDVPGLISKNIEKSLEEAFRPLSISDWNSLFWIAHPGGPAILDQVEIKLGLKPEKLKATRNVLSNYGNMSSACVLFILDEMRKASAKEGLGTTGEGLEWGVLFGFGPGLTVETVVLHSVAT

SEQ ID NO: 134 - protein, 391 aa, Arabidopsis thaliana; identical to SEQ ID NO: 101

SEQ ID NO: 135 - DNA, 43 bp, Artificial Sequence
  Primer
ctttaagaaggagatatacatatgagttcactctccaactctc

SEQ ID NO: 136 - DNA, 44 bp, Artificial Sequence
  Primer
cttgtcgacggagctcgaattcattacatgagaggcaggctgtg

SEQ ID NO: 137 - DNA, 44 bp, Artificial Sequence
  Primer
ctttaagaaggagatatacatatgagttcactctccaacgcttc

SEQ ID NO: 138 - DNA, 44 bp, Artificial Sequence; identical to SEQ ID NO: 136
  Primer

SEQ ID NO: 139 - DNA, 44 bp, Artificial Sequence
  Primer
ctttaagaaggagatatacatatgggttcactctccgactctac

SEQ ID NO: 140 - DNA, 44 bp, Artificial Sequence
  Primer
cttgtcgacggagctcgaattcattacacaggaggcaggctatg

SEQ ID NO: 141 - DNA, 42 bp, Artificial Sequence
  Primer
ctttaagaaggagatatacatatgggttcactctccaactac

SEQ ID NO: 142 - DNA, 42 bp, Artificial Sequence
  Primer
cttgtcgacggagctcgaattcattacacagggggcaggctg

SEQ ID NO: 143 - DNA, 39 bp, Artificial Sequence
  Primer
ctttaagaaggagatatacatatgggttcgatcgccgag

SEQ ID NO: 144 - DNA, 42 bp, Artificial Sequence
  Primer
cttgtcgacggagctcgaattcattagacaggaggcaggctg

SEQ ID NO: 145 - DNA, 32 bp, Artificial Sequence
  Primer.
gctcaccatcattgggctccgtggccccaatg

SEQ ID NO: 146 - DNA, 32 bp, Artificial Sequence
  Primer
cattggggccacggagcccaatgatggtgagc

SEQ ID NO: 147 - DNA, 40 bp, Artificial Sequence
  Primer
gacggatggccttttactagtgcctggggtgcctaatgag

SEQ ID NO: 148 - DNA, 45 bp, Artificial Sequence
  Primer
cgatgattaattgtcaactgctacgcctgaataagtgataataag

SEQ ID NO: 149 - DNA, 22 bp, Artificial Sequence
  Primer
gttgacaattaatcatcggctc

SEQ ID NO: 150 - DNA, 27 bp, Artificial Sequence
  Primer
actagtaaaaggccatccgtcaggatg

SEQ ID NO: 151 - DNA, 20 bp, Artificial Sequence
  Primer
gttgacaattaatcatcggc

SEQ ID NO: 152 - DNA, 20 bp, Artificial Sequence
  Primer
cgtttcacttctgagttcgg

SEQ ID NO: 153 - DNA, 46 bp, Artificial Sequence
  Primer
caatttcacacaggaaacagaattcgtgtcagtcgagactaggaag

SEQ ID NO: 154 - DNA, 44 bp, Artificial Sequence
  Primer
ctctagaggatccccgggtaccattacttgatctcgaggagaac

SEQ ID NO: 155 - DNA, 46 bp, Artificial Sequence
  Primer
caatttcacacaggaaacagaattcatgaccatttcctcacctttg

SEQ ID NO: 156 - DNA, 44 bp, Artificial Sequence
  Primer
ctctagaggatccccgggtaccattacagtggcatgttgccgtg

SEQ ID NO: 157 - DNA, 43 bp, Artificial Sequence
  Primer
gagtcgacctgcaggcatgcattgacaattaatcatcggctcg

SEQ ID NO: 158 - DNA, 25 bp, Artificial Sequence
  Primer
ctcatccgccaaaacagccaagctt

SEQ ID NO: 159 - DNA, 47 bp, Artificial Sequence
  Primer
caatttcacacaggaaacagaattcatgtcagaacgtttcccaaatg

SEQ ID NO: 160 - DNA, 45 bp, Artificial Sequence
  Primer
ctctagaggatccccgggtaccattacatcaccagacggcgaatg

SEQ ID NO: 161 - DNA, 47 bp, Artificial Sequence
  Primer
caatttcacacaggaaacagaattcatgagtactgaaatcaaaactc

SEQ ID NO: 162 - DNA, 44 bp, Artificial Sequence
  Primer
ctctagaggatccccgggtaccattacttcttcttcgctttcgg

SEQ ID NO: 163 - DNA, 43 bp, Artificial Sequence
  Primer
gtacccggggatcctctagagttgacaattaatcatcggctcg

SEQ ID NO: 164 - DNA, 25 bp, Artificial Sequence
  Primer
caagcttgcatgcctgcaggtcgac

SEQ ID NO: 165 - DNA, 49 bp, Artificial Sequence
  Primer
caatttcacacaggaaacagaattcatgactatcaaagtaggtatcaac

SEQ ID NO: 166 - DNA, 45 bp, Artificial Sequence
  Primer
ctctagaggatccccgggtaccattatttggagatgtgagcgatc

SEQ ID NO: 167 - DNA, 50 bp, Artificial Sequence
  Primer
caatttcacacaggaaacagaattcatgtctgtaattaagatgaccgatc

SEQ ID NO: 168 - DNA, 45 bp, Artificial Sequence
  Primer
ctctagaggatccccgggtaccattacttcttagcgcgctcttcg

SEQ ID NO: 169 - DNA, 46 bp, Artificial Sequence
  Primer
caatttcacacaggaaacagaattcatgagccaaattcacaaacac

SEQ ID NO: 170 - DNA, 43 bp, Artificial Sequence
  Primer
ctctagaggatccccgggtaccattacgatggcatcgcgatag

SEQ ID NO: 171 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 172 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 173 - protein, 410 aa, Escherichia coli
MAKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAAGSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSDNGSTLSAVNFPEVSLPLHGGRRLMHIHENRPGVLTALNKIFAEQGVNIAAQYLQTSAQMGYVVIDIEADEDVAEKALQAMKAIPGTIRARLLY

SEQ ID NO: 174 - protein, 331 aa, Escherichia coli
MTIKVGINGFGRIGRIVFRAAQKRSDIEIVAINDLLDADYMAYMLKYDSTHGRFDGTVEVKDGHLIVNGKKIRVTAERDPANLKWDEVGVDVVAEATGLFLTDETARKHITAGAKKVVMTGPSKDNTPMFVKGANFDKYAGQDIVSNASCTTNCLAPLAKVINDNFGIIEGLMTTVHATTATQKTVDGPSHKDWRGGRGASQNIIPSSTGAAKAVGKVLPELNGKLTGMAFRVPTPNVSVVDLTVRLEKAATYEQIKAAVKAAAEGEMKGVLGYTEDDVVSTDFNGEVCTSVFDAKAGIALNDNFVKLVSWYDNETGYSNKVLDLIAHISK

SEQ ID NO: 175 - protein, 387 aa, Escherichia coli
MSVIKMTDLDLAGKRVFIRADLNVPVKDGKVTSDARIRASLPTIELALKQGAKVMVTSHLGRPTEGEYNEEFSLLPVVNYLKDKLSNPVRLVKDYLDGVDVAEGELVVLENVRFNKGEKKDDETLSKKYAALCDVFVMDAFGTAHRAQASTHGIGKFADVACAGPLLAAELDALGKALKEPARPMVAIVGGSKVSTKLTVLDSLSKIADQLIVGGGIANTFIAAQGHDVGKSLYEADLVDEAKRLLTTCNIPVPSDVRVATEFSETAPATLKSVNDVKADEQILDIGDASAQELAEILKNAKTILWNGPVGVFEFPNFRKGTEIVANAIADSEAFSIAGGGDTLAAIDLFGIADKISYISTGGGAFLEFVEGKVLPAVAMLEERAKK

SEQ ID NO: 176 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 177 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 178 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMVFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 179 - protein, 474 aa, Escherichia coli [sequence omitted]

SEQ ID NO: 180 - protein, 591 aa, Corynebacterium glutamicum
VSVETRKITKVLVANRGEIAIRVFRAARDEGIGSVAVYAEPDADAPFVSYADEAFALGGQTSAESYLVIDKIIDAARKSGADAIHPGYGFLAENADFAEAVINEGLIWIGPSPESIRSLGDKVTARHIADTAKAPMAPGTKEPVKDAAEVVAFAEEFGLPIAIKAAFGGGGRGMKVAYKMEEVADLFESATREATAAFGRGECFVERYLDKARHVEAQVIADKHGNVVVAGTRDCSLQRRFQKLVEEAPAPFLTDDQRERLHSSAKAICKEAGYYGAGTVEYLVGSDGLISFLEVNTRLQVEHPVTEETTGIDLVREMFRIAEGHELSIKEDPAPRGHAFEFRINGEDAGSNFMPAPGKITSYREPQGPGVRMDSGVVEGSEISGQFDSMLAKLIVWGDTREQALQRSRRALAEYVVEGMPTVIPFHQHIVENPAFVGNDEGFEIYTKWIEEVWDNPIAPYVDASELDEDEDKTPAQKVVVEINGRRVEVALPGDLALGGTAGPKKKAKKRRAGGAKAGVSGDAVAAPMQGTVIKVNVEEGAEVNEGDTVVVLEAMKMENPVKAHKSGTVTGLTVAAGEGVNKGVVLLEIK

SEQ ID NO: 181 - protein, 543 aa, Corynebacterium glutamicum
MTISSPLIDVANLPDINTTAGKIADLKARRAEAHFPMGEKAVEKVHAAGRLTARERLDYLLDEGSFIETDQLARHRTTAFGLGAKRPATDGIVTGWGTIDGREVCIFSQDGTVFGGALGEVYGEKMIKIMELAIDTGRPLIGLYEGAGARIQDGAVSLDFISQTFYQNIQASGVIPQISVIMGACAGGNAYGPALTDFVVMVDKTSKMFVTGPDVIKTVTGEEITQEELGGATTHMVTAGNSHYTAATDEEALDWVQDLVSFLPSNNRSYTPLEDFDEEEGGVEENITADDLKLDEIIPDSATVPYDVRDVIECLTDDGEYLEIQADRAENVVIAFGRIEGQSVGFVANQPTQFAGCLDIDSSEKAARFVRTCDAFNIPIVMLVDVPGFLPGAGQEYGGILRRGAKLLYAYGEATVPKITVTMRKAYGGAYCVMGSKGLGSDINLAWPTAQIAVMGAAGAVGFIYRKELMAADAKGLDTVALAKSFEREYEDHMLNPYHAAERGLIDAVILPSETRGQISRNLRLLKHKNVTRPARKHGNMPL